(19) United States
(12) Patent Application Publication
(10) Pub. No.: US 2024/0083896 A1
(43) Pub. Date: Mar. 14, 2024

(54) Nitrogen Containing 2,3-Dihydroquinazolinone Compounds as Nav1.8 Inhibitors

(71) Applicant: GlaxoSmithKline Intellectual Property Development Limited, Stevenage (GB)

(72) Inventors: Xiaoyang DONG, Collegeville, PA (US); Mark Andrew ELBAN, Collegeville, PA (US); Jie GUANG, Collegeville, PA (US); Ming-Hsun HO, Collegeville, PA (US); Tram H. HOANG, Collegeville, PA (US); Joseph J. ROMANO, Collegeville, PA (US); David Glenn WASHBURN, Collegeville, PA (US)

(73) Assignee: GlaxoSmithKline Intellectual Property Development Limited, Stevenage (GB)

(21) Appl. No.: 18/256,982

(22) PCT Filed: Dec. 16, 2021
(86) PCT No.: PCT/EP2021/086098

§ 371 Date: Jun. 12, 2023

Related U.S. Application Data
(60) Provisional application No. 63/127,297, filed on Dec. 18, 2020.

(51) Int. Cl. C07D 471/04; C07D 487/04

(52) U.S. Cl. CPC C07D 471/04; C07D 487/04

(57) ABSTRACT

Compounds of formula (I) are described, wherein each of the variable groups is as defined in the specification. Also described are pharmaceutical compositions containing a compound of formula (I), and uses of the compounds and pharmaceutical compositions for inhibiting Naᵥ1.8 voltage-gated sodium channels and treating Nav1.8 mediated diseases, disorders, and conditions, such as pain and pain-associated diseases, disorders, and conditions and cardiovascular diseases, disorders, and conditions, such as atrial fibrillation.

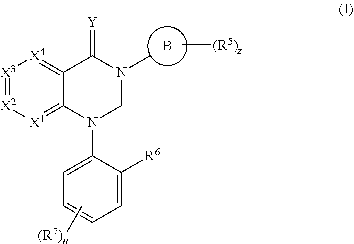

### FIELD OF THE INVENTION

The invention relates to Naᵥ1.8 inhibitor compounds or pharmaceutically acceptable salts or tautomer forms thereof, corresponding pharmaceutical compositions or formulations, methods or processes of compound preparation, methods, compounds for use in, uses for and/or combination therapies for treating pain and pain-associated diseases, disorders and conditions, and cardiovascular diseases, disorders, and conditions.

### BACKGROUND OF THE INVENTION

Pain is a protective mechanism by which animals avoid potential tissue damage, however there are numerous disease indications in which pain outlives its usefulness and becomes a disabling burden. Indications in which pain outlives its usefulness can be broadly categorized as those in which nerve damage or injury is the trigger (neuropathic pain), those in which an inflammatory response or metabolic dysregulation sensitizes the pain response (inflammatory pain) and those in which an injury or surgical procedure results in a short term elevation of pain response (post-operative/ambulatory pain).

Voltage-gated sodium channels underlie electrical signaling in all excitable tissues by setting the threshold and underlying the upstroke of action potentials. There are nine distinct isoforms of voltage-gated sodium channels. Those designated Naᵥ1.1, Naᵥ1.7, Naᵥ1.8 and Naᵥ1.9 are principally expressed on peripheral nerves where they control neuronal excitability. Naᵥ1.5 is the principle sodium channel isoform expressed in cardiac myocytes, Naᵥ1.4 is expressed and functions in skeletal muscle, whilst Naᵥ1.1, Naᵥ1.2, Naᵥ1.3 and Naᵥ1.6 are widely expressed in the central nervous system (CNS) and to an extent in the peripheral nervous system. The principal role of these nine voltage-gated sodium channels is comparable in that they control sodium influx into cells but their biophysical properties varies which greatly influences the physiological profile of their respective cell type (Catterall, 2012).

Currently, non-selective sodium channel inhibitors are utilized clinically as anti-arrhythmic and anti-seizure therapies, these include lidocaine, carbamazepine, amitriptyline and mexiletine. However, as these agents exhibit a lack of selectivity between the different sodium channel isoforms, their therapeutic utility is greatly reduced due to adverse side effects, largely mediated by activity in the CNS and heart. This has stimulated efforts to develop novel medicines which are selective for specific sodium channel isoforms in order to avoid side effects in the CNS and cardiovascular system.

The Naᵥ1.8 channel is expressed in neurons of the dorsal root ganglia (DRG) and highly expressed in the small diameter neurons of this tissue which form pain sensing C- and Aδ-nerve fibers (Abrahamsen, 2008; Amaya, 2000; Novakovic, 1998). The channel was proposed as a therapeutic target for analgesia as soon as it was originally cloned from rat DRG (Akopian, 1996) due to its prominent physiological role in this tissue type and restricted expression profile. Naᵥ1.8 was subsequently identified, cloned and characterized from human DRG tissue (Rabart 1998). The closest molecular relative of Naᵥ1.8 is Naᵥ1.5 which shares a sequence homology of ˜60%. Naᵥ1.8 was previously known as SNS (sensory neuron sodium channel), PN3 (peripheral nerve sodium channel type 3), and as it exhibits characteristic pharmacological properties in its resistant to block by tetrodotoxin, it is also described as a TTX-resistant sodium channel.

Support for Naᵥ1.8 as a therapeutic target for pain indications comes from several sources. Naᵥ1.8 has been shown to conduct the majority of current during upstroke of the action potential in DRG neurons (Blair & Bean, 2002) and due to its rate of re-priming is also critical for the ability of these neurons to fire repetitively (Blair and Bean, 2003). Increased expression and function of Naᵥ1.8 has been reported in response to painful stimuli such as inflammatory mediators (England 1996 & Gold 1996), nerve damage (Roza 2003 & Ruangsri 2011), and within painful neuromas (Black 2008 & Coward 2000). Knockout of the gene encoding Nav1.8 in mice resulted in a reduced pain phenotype in particular to inflammatory challenges (Akopian 1999). Knockdown of the mRNA encoding Naᵥ1.8 also resulted in reduced painful phenotypes in rodent models, particularly in neuropathic models (Lai 2002). Pharmacological intervention via selective small molecule inhibitors has demonstrated efficacy in rodent models of inflammatory pain as well as neuropathic pain (Jarvis 2007 & Payne 2015). Supporting genetic evidence for Naᵥ1.8 is also present in patients with chronic neuropathic pain where multiple gain of function mutations has been reported to be causative in episodic painful neuropathies and small fiber neuropathies (Faber 2012, Han 2014 & Eijkenboom 2018).

### SUMMARY OF THE INVENTION

Accordingly, there is a need for the development of novel compounds, particularly Naᵥ1.8 inhibitor compounds for use in the treatment of pain and pain associated diseases, disorders, and conditions and cardiovascular diseases, disorders and conditions. The invention satisfies this need by providing compounds with Naᵥ1.8 inhibitory activity and uses of such compounds in the treatment of pain and pain associated diseases, disorders, and conditions, and cardiovascular diseases, disorders, and conditions.

In one aspect, the invention relates to a compound of formula (I):

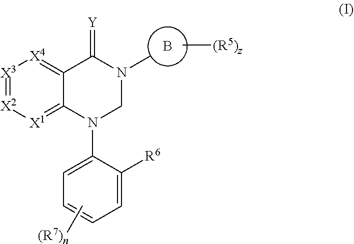

or a tautomer thereof, or a pharmaceutically acceptable salt thereof,
wherein:
   Y is O or S;
   X¹ is nitrogen or CR¹,
   X² is nitrogen or CR²,
   X³ is nitrogen or CR³, and
   X⁴ is nitrogen or CR⁴,
   provided at least one of X², X³, and X⁴ is nitrogen;
   each of R¹, R², R³, and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl; ring B is phenyl, or a 5- or 6-membered heterocyclyl containing 1 or 2 nitrogen ring atoms;
   each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
   R⁶ is hydrogen, —(C₁₋₆)alkyl, —O—(C₁₋₆)-alkyl, or —NR^{a}R^{b};
   each R⁷ is independently halo, —(C₁₋₆)alkyl, —O—(C₁₋₆)alkyl, or —O—(C₁₋₆)-haloalkyl;
   each of R^{a} and R^{b} is independently hydrogen, —(C₁₋₆)alkyl, or —(C₁₋₆)haloalkyl;
   R^{c} is hydrogen, —OH, —NR^{a}R^{b}, —(C₁₋₆)-alkyl, or —(C₁₋₆)-haloalkyl;
   n is 0, 1, 2, or 3;
   p is 0, 1, or 2; and
   z is 0, 1, 2, or 3.

In one aspect, the invention relates to a pharmaceutical composition comprising a compound or a tautomer thereof, or a pharmaceutically acceptable salt thereof as defined herein, and a pharmaceutically acceptable excipient.

In one aspect, the invention relates to a method of inhibiting a Naᵥ1.8 voltage-gated sodium channel in a subject in need thereof, the method comprising administering to the subject a compound or a tautomer thereof, or a pharmaceutically acceptable salt thereof as defined herein or a pharmaceutical composition as defined herein.

In one aspect, the invention relates to a method of treatment of pain or a pain-associated disease, disorder, or condition in a subject in need thereof, the method comprising administering to the subject a therapeutically effective amount of a compound, or a tautomer thereof, or a pharmaceutically acceptable salt thereof as defined herein or a pharmaceutical composition as defined herein.

In one aspect, the invention relates to a method of treatment of atrial fibrillation in a subject in need thereof, the method comprising administering to the subject a therapeutically effective amount of a compound, or a tautomer thereof, or a pharmaceutically acceptable salt thereof as defined herein or a pharmaceutical composition as defined herein.

In one aspect, the invention relates to a compound, or a tautomer thereof, or a pharmaceutically acceptable salt thereof as defined herein or a pharmaceutical composition as defined herein for use in treatment of pain or a pain-associated disease, disorder, or condition.

In one aspect, the invention relates to a compound, or a tautomer thereof, or a pharmaceutically acceptable salt thereof as defined herein or a pharmaceutical composition as defined herein for use in treatment of atrial fibrillation.

In one aspect, the invention relates to use of a compound, or a tautomer thereof, or a pharmaceutically acceptable salt thereof as defined herein or a pharmaceutical composition as defined herein in the manufacture of a medicament for treatment of pain or a pain-associated disease, disorder, or condition.

In one aspect, the invention relates to use of a compound, or a tautomer thereof, or a pharmaceutically acceptable salt thereof as defined herein or a pharmaceutical composition as defined herein in the manufacture of a medicament for treatment of atrial fibrillation. In one aspect, the invention relates to a compound, or a tautomer thereof, or pharmaceutically acceptable salt thereof as defined herein, or a pharmaceutical composition as defined herein for use in therapy.

### BRIEF DESCRIPTION OF THE FIGURES

FIG. 1 shows an X-ray powder diffraction (XRPD) pattern for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 1).

FIG. 2 shows a DSC for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 1).

FIG. 3 shows an X-ray powder diffraction (XRPD) pattern for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 2).

FIG. 4 shows a DSC for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 2).

FIG. 5 shows an X-ray powder diffraction (XRPD) pattern for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 3).

FIG. 6 shows an X-ray powder diffraction (XRPD) pattern for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 4).

FIG. 7 shows a DSC for 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 4).

### DETAILED DESCRIPTION OF THE INVENTION

Various publications, articles and patents are cited or described in the background and throughout the specification. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, the recitation of “10-fold” includes 9-fold and 11-fold. As used herein, the use of a numerical range expressly includes all possible subranges, all individual numerical values within that range, including integers within such ranges and fractions of the values unless the context clearly indicates otherwise.

The present invention relates to Naᵥ1.8 Inhibitor compounds of Formula (I) or pharmaceutically acceptable salts or tautomer forms thereof, corresponding pharmaceutical compositions, methods or processes of compound preparation, methods, compounds for use in, uses for and/or combination therapies for treating Nav1.8 mediated diseases, disorders, and conditions, such as pain and/or pain-associated disease(s), disorder(s) or condition(s), respectively, and atrial fibrillation.

The definitions for the various groups and substituent groups of any of the Formulas disclosed herein, or a pharmaceutically acceptable salt and/or a corresponding tautomer form thereof provided throughout the specification are intended to particularly describe each compound species disclosed herein, individually, as well as groups of one or more compound species.

As used herein, the term alkali metal is intended to mean the Group I elements, which include, but are not limited to lithium (Li), sodium (Na), or potassium (K) and the like. The term alkali earth metal may include, but are not limited to calcium (Ca) or magnesium (Mg) and the like.

As used herein, the terms “alkyl” or “straight or branched alkyl”, and the like, represent a saturated, straight or branched hydrocarbon moiety. Exemplary alkyls include, but are not limited to methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When a number appears in a subscript after the symbol “C,” the subscript defines with more specificity the number of carbon atoms which that particular alkyl can contain. For example, the terms “C₁-C₆” and “C₁₋₆” refer to an alkyl containing 1 to 6 carbon atoms and the terms “C₁-C₄” and “C₁₋₄” refer to an alkyl containing 1 to 4 carbon atoms.

When the term “alkyl” is used in combination with other substituent groups, such as “haloalkyl” or “hydroxyalkyl”, the term “alkyl” is intended to encompass a divalent saturated, straight or branched-chain hydrocarbon radical.

For example, the terms “haloalkyl” or “straight or branched haloalkyl” are intended to mean a saturated, straight or branched hydrocarbon moiety substituted with one or more halogens, where halogen is independently selected from: fluoro, chloro, bromo and iodo. A haloalkyl group can have a specified number of carbon atoms. For example, the terms “(C₁-C₆)haloalkyl” and “(C₁₋₆)haloalkyl” refer to a saturated, straight- or branched-chain haloalkyl radical, having at least 1 and up to 6 carbon atoms. Likewise, the terms “(C₁. C₄)haloalkyl” and “(C₁₋₄)haloalkyl” refer to a saturated, straight- or branched-chain haloalkyl radical having 1 to 4 carbon atoms. “Fluorinated alkyl” or “fluoroalkyl” in particular refers to any alkyl group as defined above substituted with at least one fluoro atom, e.g., one to three fluoro atoms, such as one, two, or three fluoroatoms. Representative haloalkyls include, but are not limited to trifluoromethyl (—CF₃), tetrafluoroethyl (—CF₂CHF₂), pentafluoroethyl (—CF₂CF₃) and the like.

The term “hydroxyalkyl” refers to a saturated, straight or branched hydrocarbon moiety substituted with one or more hydroxy groups.

As used herein, the terms “halogen” and “halo” mean fluoro (—F), chloro (—Cl), bromo (—Br), and iodo (—I).

“Hydroxy” or “hydroxyl” is intended to mean the radical —OH.

“Oxo” represents a double-bonded oxygen moiety; for example, if attached directly to a carbon atom forms a carbonyl moiety (C═O), or attached to an N or S forms oxides, e.g., N-oxides, sulfones or sulfoxides.

The term “cyano” refers to —CN.

The term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

“Alkoxy” refers to a group containing an alkyl radical attached through an oxygen linking atom, wherein alkyl is as defined above. An alkoxy group can have a specified number of carbon atoms. For example, the terms “(C₁-C₆)alkoxy” and “(C₁₋₆)alkoxy” refer to an alkyl radical, having at least 1 and up to 6 carbon atoms attached through an oxygen linking atom. Likewise, the terms “(C₁-C₄)alkoxy” and “(C₁₋₄)alkoxy” refer to an alkyl radical having at least 1 and up to 4 carbon atoms attached through an oxygen linking atom. Exemplary alkoxy groups include, but are not limited to, methoxy, ethoxy, n-propoxy, isopropoxy, n-butoxy, s-butoxy, and t-butoxy.

“Haloalkoxy” refers to an alkoxy group in which the alkyl moiety is substituted with one or more halogens, wherein halogen is independently selected from fluoro, chloro, bromo, and iodo. A haloalkoxy group can have a specified number of carbon atoms. For example, the term “(C₁-C₆)haloalkoxy refers to a haloalkyl radical, having at 1 to 6 carbon atoms attached through an oxygen linking atom. Representative haloalkoxy groups include, but are not limited to difluoromethoxy (—OCHCF₂), trifluoromethoxy (—OCF₃), tetrafluoroethoxy (—OCF₂CHF₂) and the like.

“Aryl” represents an aromatic hydrocarbon ring. Aryl groups are monocyclic, bicyclic, and tricyclic ring systems having a total of five to fourteen ring member atoms, wherein at least one ring system is aromatic and wherein each ring in the system contains 3 to 7 member atoms, such as phenyl, naphthalene, and tetrahydronaphthalene. Suitably aryl is phenyl.

“Heteroatoms” are defined as oxygen, nitrogen, sulfur and the like. Suitably, “heteroatom” refers to a nitrogen, sulfur or oxygen atom.

“Heterocyclyl” includes heteroaryl and heterocycloalkyl groups. Heterocyclyl groups may be unsaturated or saturated. Unless otherwise stated, monocyclic heterocyclyl rings have from 3 to 7 ring atoms and contains up to four heteroatoms. Monocyclic heterocyclyl rings or fused heterocyclyl rings include substituted aromatic and non-aromatics.

“Heterocycloalkyl” represents a group or moiety comprising a non-aromatic, monovalent monocyclic or bicyclic radical, which is saturated or partially unsaturated. Unless otherwise stated, heterocycloalkyls contain 3 to 10 ring atoms, which includes 1 to 4 heteroatoms independently selected from nitrogen, oxygen and sulfur, and which may be unsubstituted or substituted by one or more specified substituent groups. Generally, in the compounds of this invention, heterocycloalkyl groups are 5-membered and/or 6-membered heterocycloalkyl groups.

“Heteroaryl” represents a group or moiety comprising an aromatic monovalent monocyclic or bicyclic radical. Unless otherwise stated, heteroaryls contain 4 to 10 ring atoms, suitably 5 to 10 ring atoms, including 1 to 4 heteroatoms independently selected from nitrogen, oxygen and sulfur, which may be unsubstituted or substituted by one or more specified substituent groups. “Heteroaryl” also encompasses bicyclic heterocyclic-aryl compounds containing an aryl ring moiety fused to a heterocycloalkyl ring moiety, containing 4 to 10 ring atoms, suitably containing 5 to 10 ring atoms, including 1 to 4 heteroatoms independently selected from nitrogen, oxygen and sulfur, which may be unsubstituted or substituted by one or more of the substituents defined herein. Heteroaryl includes but is not limited to: benzoimidazolyl, benzothiazolyl, benzothiophenyl, benzopyrazinyl, benzotriazolyl, benzotriazinyl, benzo[1,4]dioxanyl, benzofuranyl, 9H-a-carbolinyl, cinnolinyl, furanyl, pyrazolyl, imidazolyl, indolizinyl, naphthyridinyl, oxazolyl, oxothiadiazolyl, oxadiazolyl, phthalazinyl, pyridyl (or pyridinyl), pyrrolyl, purinyl, pteridinyl, phenazinyl, pyrazinyl, pyrazolopyrimidinyl, pyrazolopyridinyl, pyrrolizinyl, pyrimidyl, isothiazolyl, furazanyl, pyrimidinyl, tetrazinyl, isoxazolyl, quinoxalinyl, quinazolinyl, quinolinyl, quinolizinyl, thienyl, thiophenyl, triazolyl, triazinyl, tetrazolopyrimidinyl, triazolopyrimidinyl, tetrazolyl, thiazolyl and thiazolidinyl.

Heteroaryl groups present in the compounds of this invention are typically 5-membered and/or 6-membered monocyclic heteroaryl groups containing 1 or 2 nitrogen ring atoms. Exemplary 5-membered and/or 6-membered monocyclic heteroaryl groups containing 1 or 2 nitrogen ring atoms include, but are not limited to, pyridyl (or pyridinyl), pyrazinyl, pyrimidinyl, pyridazinyl, pyrrolyl, imidazolyl, pyrazolyl and the like.

In one embodiment, a heteroaryl group is pyridinyl optionally substituted by one or more of the defined substituent groups, such as oxo, halo, alkyl, etc. For example, pyridinyl can be substituted by oxo to form a pyridone ring moiety, which may include, but are not limited to: -3-pyridonyl, -4-pyridonyl, -5-pyridonyl, and the like. Heteroaryl also encompasses pyridazinyl, pyrimidinyl, and pyrazinyl substituted with oxo, including, but not limited to, those moieties shown below, which may be optionally substituted by one or more additional specified substituent groups:

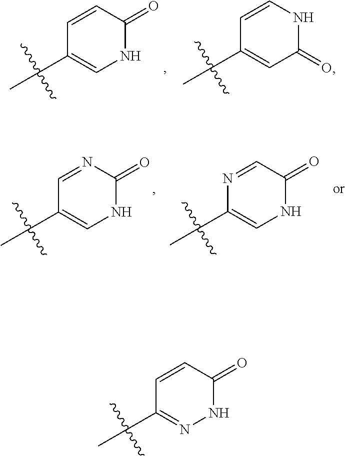 [SMILES: *c1ccc(=O)[nH]c1 ; *c1cc[nH]c(=O)c1 ; *c1cnc(=O)[nH]c1 ; *c1c[nH]c(=O)cn1 ; *c1ccc(=O)[nH]n1]

and the like.

In accordance with convention used in the art:

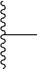

is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

As used herein, the term “compound(s) of the invention” means a compound of any of the Formulas disclosed herein, in any form, i.e., any salt or non-salt form (e.g., as a free acid or base form, or as a pharmaceutically acceptable salt thereof), any tautomer form thereof, and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means that a group (e.g., alkyl, etc.), may be unsubstituted, or the group may be substituted with one or more substituent(s) as defined herein throughout the instant specification. The term “substituted” as used herein with respect to a group (e.g., alkyl, etc.) means that at least one hydrogen atom is replaced with a non-hydrogen group, provided that all normal valencies are maintained and that the substitution results in a stable compound. In the case where groups may be selected from a number of alternative groups the selected groups may be the same or different. For example, various substituent groups of compound formulas as defined in the present invention may be optionally substituted, but are not limited to substituents, such as halo, cyano, amino, alkyl, haloalkyl, alkoxy, and the like.

The term “independently” when used with reference to a substituent or heteroatom means that where more than one substituent or heteroatom is selected from a number of possible substituents or heteroatoms, respectively, those substituents or heteroatoms may be the same or different.

Compounds

In particular, the present invention relates to novel Naᵥ1.8 inhibitor compounds of any of the Formulas disclosed herein, or a pharmaceutically acceptable salt and/or a corresponding tautomer form thereof.

In one aspect, the present invention relates to a compound of Formula (I):

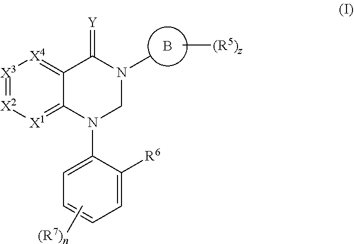

or a tautomer thereof, or a pharmaceutically acceptable salt thereof,
   wherein:
      Y is O or S;
      X¹ is nitrogen or CR¹,
      X² is nitrogen or CR²,
      X³ is nitrogen or CR³, and
      X⁴ is nitrogen or CR⁴,
      provided at least one of X², X³, and X⁴ is nitrogen;
      each of R¹, R², R³, and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
      ring B is phenyl, or a 5- or 6-membered heterocyclyl containing 1 or 2 nitrogen ring atoms;
      each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
      R⁶ is hydrogen, —(C₁₋₆)alkyl, —O—(C₁₋₆)-alkyl, or —NR^{a}R^{b};
      each R⁷ is independently halo, —(C₁₋₆)alkyl, —O—(C₁₋₆)alkyl, or —O—(C₁₋₆)-haloalkyl;
      each of R^{a} and R^{b} is independently hydrogen, —(C₁₋₆)alkyl, or —(C₁₋₆)haloalkyl;
      R^{c} is hydrogen, —OH, —NR^{a}R^{b}, —(C₁₋₆)-alkyl, or —(C₁₋₆)-haloalkyl;
      n is 0, 1, 2, or 3;
      p is 0, 1, or 2; and
      z is 0, 1, 2, or 3.

In an embodiment of a compound of formula (I), or a tautomer thereof, or a pharmaceutically acceptable salt thereof:
Y is O or S;
X¹ is nitrogen or CR¹,
X² is nitrogen or CR²,
X³ is nitrogen or CR³, and
X⁴ is nitrogen or CR⁴,
provided at least one of X², X³, and X⁴ is nitrogen;
each of R¹, R², R³, and R⁴ is independently hydrogen, halo, cyano, —(C₁₋₆)-alkyl, —(C₁₋₆)-haloalkyl, —O—(C₁₋₆)— alkyl, or —O—(C₁₋₆)-haloalkyl; ring B is a 5- or 6-membered heteroaryl containing 1 or 2 nitrogen ring atoms;
each R⁵ is independently selected from the group consisting of halo, oxo, —OH, —NR^{a}R^{b}, or —(C₁₋₆)alkyl;
R⁶ is —(C₁₋₆)alkyl;
each R⁷ is independently halo, —(C₁₋₆)alkyl, —O—(C₁₋₆)alkyl, or —O—(C₁₋₆)-haloalkyl; each of R^{a} and R^{b} is independently hydrogen, —(C₁₋₆)alkyl, or —(C₁₋₆)haloalkyl;
n is 0, 1, 2, or 3; and
z is 0, 1, 2, or 3.

In an embodiment of a compound of formula (I), or a tautomer thereof, or a pharmaceutically acceptable salt thereof, Y is O.

In an embodiment of a compound of formula (I), or a tautomer thereof, or a pharmaceutically acceptable salt thereof, Y is S.

In an embodiment of a compound of formula (I), or a tautomer thereof, or a pharmaceutically acceptable salt thereof, X¹ is nitrogen.

In an embodiment of a compound of formula (I), or a tautomer thereof, or a pharmaceutically acceptable salt thereof, X² is nitrogen.

In an embodiment of a compound of formula (I), or a tautomer thereof, or a pharmaceutically acceptable salt thereof, X³ is nitrogen.

In an embodiment of a compound of formula (I), or a tautomer thereof, or a pharmaceutically acceptable salt thereof, X⁴ is nitrogen.

In an embodiment of a compound of formula (I), or a tautomer thereof, or a pharmaceutically acceptable salt thereof, X¹ is CR¹.

In an embodiment of a compound of formula (I), or a tautomer thereof, or a pharmaceutically acceptable salt thereof, X² is CR².

In an embodiment of a compound of formula (I), or a tautomer thereof, or a pharmaceutically acceptable salt thereof, X³ is CR³.

In an embodiment of a compound of formula (I), or a tautomer thereof, or a pharmaceutically acceptable salt thereof, X⁴ is CR⁴.

In an embodiment of a compound of formula (I), or a tautomer thereof, or a pharmaceutically acceptable salt thereof, at least one of X², X³, and X⁴ is nitrogen and no more than two of X¹, X², X³, and X⁴ is nitrogen.

In an embodiment of a compound of formula (I), or a tautomer thereof, or a pharmaceutically acceptable salt thereof, X¹ is CR¹ and R¹ is hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl.

In an embodiment of a compound of formula (I), or a tautomer thereof, or a pharmaceutically acceptable salt thereof, X¹ is CR¹ and R¹ is hydrogen, halo, cyano, or —(C₁₋₆)-haloalkyl.

In an embodiment of a compound of formula (I), or a tautomer thereof, or a pharmaceutically acceptable salt thereof, X¹ is CR¹ and R¹ is hydrogen.

In an embodiment of a compound of formula (I), or a tautomer thereof, or a pharmaceutically acceptable salt thereof, X² is CR² and R² is hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl.

In an embodiment of a compound of formula (I), or a tautomer thereof, or a pharmaceutically acceptable salt thereof, X² is CR² and R² is hydrogen, halo, cyano, or —(C₁₋₆)-haloalkyl.

In an embodiment of a compound of formula (I), or a tautomer thereof, or a pharmaceutically acceptable salt thereof, X² is CR² and R² is hydrogen, —CF₃, —Cl, or cyano.

In an embodiment of a compound of formula (I), or a tautomer thereof, or a pharmaceutically acceptable salt thereof, X³ is CR³ and R³ is hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl.

In an embodiment of a compound of formula (I), or a tautomer thereof, or a pharmaceutically acceptable salt thereof, X³ is CR³ and R³ is hydrogen, halo, cyano, or —(C₁₋₆)-haloalkyl.

In an embodiment of a compound of formula (I), or a tautomer thereof, or a pharmaceutically acceptable salt thereof, X³ is CR³ and R³ is hydrogen, —CF₃, —Cl, or cyano.

In an embodiment of a compound of formula (I), or a tautomer thereof, or a pharmaceutically acceptable salt thereof, X⁴ is CR⁴ and R⁴ is hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl.

In an embodiment of a compound of formula (I), or a tautomer thereof, or a pharmaceutically acceptable salt thereof, X⁴ is CR⁴ and R⁴ is hydrogen, halo, cyano, or —(C₁₋₆)-haloalkyl.

In an embodiment of a compound of formula (I), or a tautomer thereof, or a pharmaceutically acceptable salt thereof, X⁴ is CR⁴ and R⁴ is hydrogen.

In an embodiment of a compound of formula (I), or a tautomer thereof, or a pharmaceutically acceptable salt thereof, X¹ is CR¹, X² is CR², X³ is CR³, and X⁴ is nitrogen.

In an embodiment of a compound of formula (I), or a tautomer thereof, or a pharmaceutically acceptable salt thereof, X¹ is CR¹, X² is nitrogen, X³ is CR³, and X⁴ is CR⁴.

In an embodiment of a compound of formula (I), or a tautomer thereof, or a pharmaceutically acceptable salt thereof, X¹ is CR¹, X² is CR², X³ is nitrogen, and X⁴ is CR⁴.

In an embodiment of a compound of formula (I), or a tautomer thereof, or a pharmaceutically acceptable salt thereof, X¹ is nitrogen, X² is CR², X³ is nitrogen, and X⁴ is CR⁴.

In an embodiment of a compound of formula (I), or a tautomer thereof, or a pharmaceutically acceptable salt thereof, X¹ is nitrogen, X² is CR², X³ is CR³, and X⁴ is nitrogen.

In an embodiment of a compound of formula (I), or a tautomer thereof, or a pharmaceutically acceptable salt thereof, ring B is phenyl.

In an embodiment of a compound of formula (I), or a tautomer thereof, or a pharmaceutically acceptable salt thereof, ring B is a 5- or 6-membered heterocyclyl containing 1 or 2 nitrogen ring atoms.

In an embodiment of a compound of formula (I), or a tautomer thereof, or a pharmaceutically acceptable salt thereof, ring B is a 5- or 6-membered heteroaryl containing 1 or 2 nitrogen ring atoms.

In an embodiment of a compound of formula (I), or a tautomer thereof, or a pharmaceutically acceptable salt thereof, ring B is a 5- or 6-membered heterocycloalkyl containing 1 or 2 nitrogen ring atoms.

In an embodiment of a compound of formula (I), or a tautomer thereof, or a pharmaceutically acceptable salt thereof, each R⁵ is independently halo, oxo, —OH, —NR^{a}R^{b}, or —(C₁₋₆)alkyl.

In an embodiment of a compound of formula (I), or a tautomer thereof, or a pharmaceutically acceptable salt thereof, ring B is a 5- or 6-membered heteroaryl containing 1 or 2 nitrogen ring atoms; and each R⁵ is independently halo, oxo, —OH, —NR^{a}R^{b}, —(C₁₋₆)alkyl, or —(C₁₋₆)haloalkyl.

In an embodiment of a compound of formula (I), or a tautomer thereof, or a pharmaceutically acceptable salt thereof, ring B is pyridinyl; and each R⁵ is independently halo, oxo, —OH, —NR^{a}R^{b}, or —(C₁₋₆)alkyl.

In an embodiment of a compound of formula (I), or a tautomer thereof, or a pharmaceutically acceptable salt thereof, ring B is pyridinyl; and each R⁵ is independently oxo or —(C₁₋₆)alkyl.

In an embodiment of a compound of formula (I), or a tautomer thereof, or a pharmaceutically acceptable salt thereof, ring B is pyridinyl; and each R⁵ is independently oxo, —F, —Cl, —NH₂, or —CH₃.

In an embodiment of a compound of formula (I), or a tautomer thereof, or a pharmaceutically acceptable salt thereof, ring B is pyridinyl; and each R⁵ is independently oxo, —F, —NH₂, or —CH₃.

In an embodiment of a compound of formula (I), or a tautomer thereof, or a pharmaceutically acceptable salt thereof, ring B is pyridinyl; and each R⁵ is independently oxo, —F or —CH₃.

In an embodiment of a compound of formula (I), or a tautomer thereof, or a pharmaceutically acceptable salt thereof, ring B is pyridinyl; and each R⁵ is independently oxo or —CH₃.

In an embodiment of a compound of formula (I), or a tautomer thereof, or a pharmaceutically acceptable salt thereof, ring B is pyrazolyl; and each R⁵ is independently halo, oxo, —OH, —NR^{a}R^{b}, or —(C₁₋₆)alkyl.

In an embodiment of a compound of formula (I), or a tautomer thereof, or a pharmaceutically acceptable salt thereof, ring B is pyrazolyl; and each R⁵ is independently —(C₁₋₆)alkyl.

In an embodiment of a compound of formula (I), or a tautomer thereof, or a pharmaceutically acceptable salt thereof, ring B is pyrazolyl; and each R⁵ is independently —CH₃.

In an embodiment of a compound of formula (I), or a tautomer thereof, or a pharmaceutically acceptable salt thereof, z is 1, 2, or 3 and each R⁵ is independently —CH₃, —F, —Cl, oxo, or —NH₂.

In an embodiment of a compound of formula (I), or a tautomer thereof, or a pharmaceutically acceptable salt thereof,

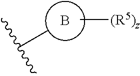

is:

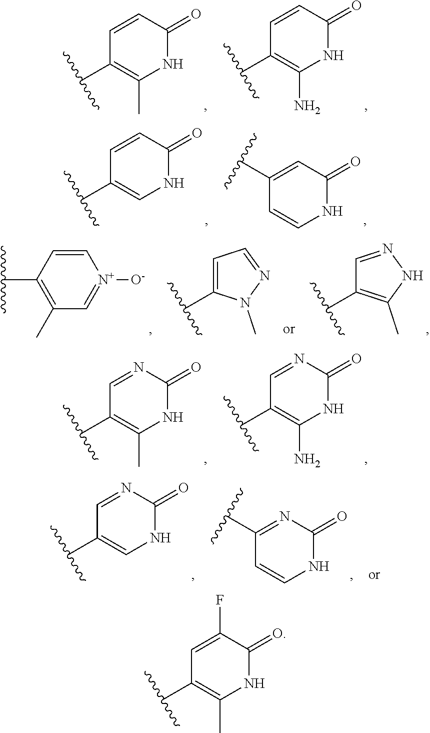

In an embodiment of a compound of formula (I), or a tautomer thereof, or a pharmaceutically acceptable salt thereof,

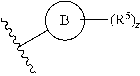

is:

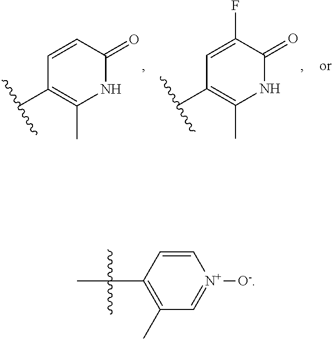

In an embodiment of a compound of formula (I), or a tautomer thereof, or a pharmaceutically acceptable salt thereof,

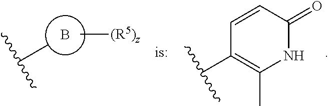

In an embodiment of a compound of formula (I), or a tautomer thereof, or a pharmaceutically acceptable salt thereof, R⁶ is hydrogen.

In an embodiment of a compound of formula (I), or a tautomer thereof, or a pharmaceutically acceptable salt thereof, R⁶ is —(C₁₋₆)alkyl.

In an embodiment of a compound of formula (I), or a tautomer thereof, or a pharmaceutically acceptable salt thereof, R⁶ is —CH₃, —CH₂CH₃, or —CH(CH₃)₂.

In an embodiment of a compound of formula (I), or a tautomer thereof, or a pharmaceutically acceptable salt thereof, R⁶ is —OH.

In an embodiment of a compound of formula (I), or a tautomer thereof, or a pharmaceutically acceptable salt thereof, R⁶ is —NR^{a}R^{b}.

In an embodiment of a compound of formula (I), or a tautomer thereof, or a pharmaceutically acceptable salt thereof, R⁶ is —NH₂.

In an embodiment of a compound of formula (I), or a tautomer thereof, or a pharmaceutically acceptable salt thereof, each R⁷ is independently halo or —O(C₁₋₆)haloalkyl.

In an embodiment of a compound of formula (I), or a tautomer thereof, or a pharmaceutically acceptable salt thereof, each R⁷ is independently —F or —OCF₃.

In an embodiment of a compound of formula (I), or a tautomer thereof, or a pharmaceutically acceptable salt thereof, n is 1 or 2; and each R⁷ is independently —F or —OCF₃.

In an embodiment of a compound of formula (I), or a tautomer thereof, or a pharmaceutically acceptable salt thereof, n is 1 and R⁷ is —F.

In an embodiment of a compound of formula (I), or a tautomer thereof, or a pharmaceutically acceptable salt thereof, n is 1 and R⁷ is —OCF₃.

In an embodiment of a compound of formula (I), or a tautomer thereof, or a pharmaceutically acceptable salt thereof, n is 2 and each R⁷ is —F.

In an embodiment of a compound of formula (I), or a tautomer thereof, or a pharmaceutically acceptable salt thereof, R⁶ is —CH₃, —CH₂CH₃, or —CH(CH₃)₂; n is 1 or 2; and each R⁷ is independently —F or —OCF₃.

In an embodiment of a compound of formula (I), or a tautomer thereof, or a pharmaceutically acceptable salt thereof,

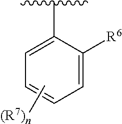

has the structure:

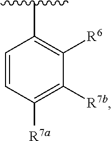

wherein each of R^{7a} and R^{7b} is independently hydrogen, halo, —(C₁₋₆)alkyl, —O—(C₁₋₆)alkyl, or —O—(C₁₋₆)-haloalkyl.

In an embodiment of a compound of formula (I), or a tautomer thereof, or a pharmaceutically acceptable salt thereof, each of R^{7a} and R^{7b} is independently hydrogen, halo, or —O(C₁₋₆)haloalkyl.

In an embodiment of a compound of formula (I), or a tautomer thereof, or a pharmaceutically acceptable salt thereof, R^{7a} is —F or —OCF₃.

In an embodiment of a compound of formula (I), or a tautomer thereof, or a pharmaceutically acceptable salt thereof, R^{7b} is hydrogen or —F.

In an embodiment of a compound of formula (I), or a tautomer thereof, or a pharmaceutically acceptable salt thereof, R^{7a} is —F or —OCF₃; and R^{7b} is hydrogen or —F.

In an embodiment of a compound of formula (I), or a tautomer thereof, or a pharmaceutically acceptable salt thereof, R^{7a} is —F; and R^{7b} is hydrogen.

In an embodiment of a compound of formula (I), or a tautomer thereof, or a pharmaceutically acceptable salt thereof,

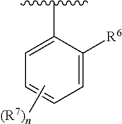

is:

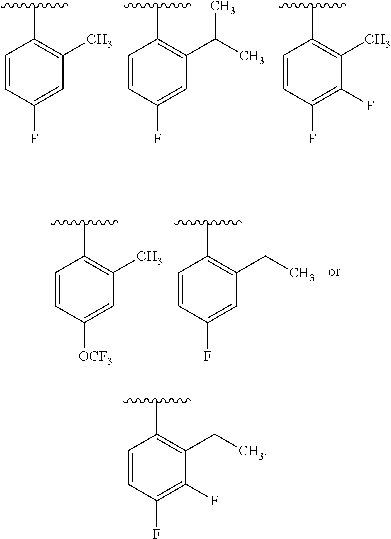

In an embodiment of a compound of formula (I), or a tautomer thereof, or a pharmaceutically acceptable salt thereof, each of R^{a} and R^{b} is independently hydrogen or —(C₁₋₆)alkyl.

In an embodiment of a compound of formula (I), or a tautomer thereof, or a pharmaceutically acceptable salt thereof:
Y is O;
X¹, X², X³ and X⁴ are as defined for formula (I);
each of R¹, R², R³, and R⁴ is independently hydrogen, —CF₃, Cl, or cyano;
ring B is a 5- or 6-membered heteroaryl containing 1 or 2 nitrogen ring atoms;
each R⁵ is independently oxo, —F, —CH₃, or —NH₂;
R⁶ is —CH₃, —CH₂CH₃, or —CH(CH₃)₂;
each R⁷ is independently —F or —OCF₃;
z is 0, 1, 2, or 3; and
n is 1 or 2.

In another aspect, the invention relates to a compound of Formula (I-A):

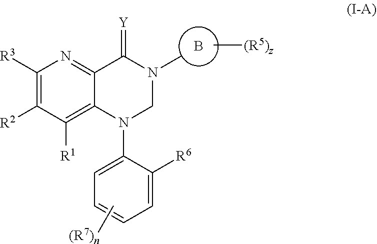

or a pharmaceutically acceptable salt or tautomer thereof,
wherein:
Y is O or S;
each of R¹, R², and R³ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

In an embodiment of a compound of formula (I-A), or a tautomer thereof, or a pharmaceutically acceptable salt thereof,
Y is O or S;
each of R¹, R², and R³ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is a 5- or 6-membered heteroaryl containing 1 or 2 nitrogen ring atoms;
each R⁵ is independently halo, oxo, —OH, —NR^{a}R^{b}, or —(C₁₋₆)alkyl;
R⁶ is —(C₁₋₆)alkyl;
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
n is 0, 1, 2, or 3; and
z is 0, 1, 2, or 3.

In an embodiment of a compound of formula (I-A), or a tautomer thereof, or a pharmaceutically acceptable salt thereof, Y is O.

In an embodiment of a compound of formula (I-A), or a tautomer thereof, or a pharmaceutically acceptable salt thereof, Y is S.

In an embodiment of a compound of formula (I-A), or a tautomer thereof, or a pharmaceutically acceptable salt thereof, R¹ is hydrogen, halo, cyano, or —(C₁₋₆)-haloalkyl.

In an embodiment of a compound of formula (I-A), or a tautomer thereof, or a pharmaceutically acceptable salt thereof, R¹ is hydrogen, —CF₃, —Cl, or cyano.

In an embodiment of a compound of formula (I-A), or a tautomer thereof, or a pharmaceutically acceptable salt thereof, R¹ is hydrogen.

In an embodiment of a compound of formula (I-A), or a tautomer thereof, or a pharmaceutically acceptable salt thereof, R² is hydrogen, halo, cyano, or —(C₁₋₆)-haloalkyl.

In an embodiment of a compound of formula (I-A), or a tautomer thereof, or a pharmaceutically acceptable salt thereof, R² is hydrogen, —CF₃, —Cl, or cyano.

In an embodiment of a compound of formula (I-A), or a tautomer thereof, or a pharmaceutically acceptable salt thereof, R³ is hydrogen, halo, cyano, or —(C₁₋₆)-haloalkyl.

In an embodiment of a compound of formula (I-A), or a tautomer thereof, or a pharmaceutically acceptable salt thereof, R³ is hydrogen, —CF₃, —Cl, or cyano.

In an embodiment of a compound of formula (I-A), or a tautomer thereof, or a pharmaceutically acceptable salt thereof, ring B is phenyl.

In an embodiment of a compound of formula (I-A), or a tautomer thereof, or a pharmaceutically acceptable salt thereof, ring B is a 5- or 6-membered heterocyclyl containing 1 or 2 nitrogen ring atoms.

In an embodiment of a compound of formula (I-A), or a tautomer thereof, or a pharmaceutically acceptable salt thereof, ring B is a 5- or 6-membered heteroaryl containing 1 or 2 nitrogen ring atoms.

In an embodiment of a compound of formula (I-A), or a tautomer thereof, or a pharmaceutically acceptable salt thereof, ring B is a 5- or 6-membered heterocycloalkyl containing 1 or 2 nitrogen ring atoms.

In an embodiment of a compound of formula (I-A), or a tautomer thereof, or a pharmaceutically acceptable salt thereof, each R⁵ is independently halo, oxo, —OH, —NR^{a}R^{b}, or —(C₁₋₆)alkyl.

In an embodiment of a compound of formula (I-A), or a tautomer thereof, or a pharmaceutically acceptable salt thereof, ring B is a 5- or 6-membered heteroaryl containing 1 or 2 nitrogen ring atoms; and each R⁵ is independently halo, oxo, —OH, —NR^{a}R^{b}, —(C₁₋₆)alkyl, or —(C₁₋₆)haloalkyl.

In an embodiment of a compound of formula (I-A), or a tautomer thereof, or a pharmaceutically acceptable salt thereof, ring B is pyridinyl; and each R⁵ is independently halo, oxo, —OH, —NR^{a}R^{b}, or —(C₁₋₆)alkyl.

In an embodiment of a compound of formula (I-A), or a tautomer thereof, or a pharmaceutically acceptable salt thereof, ring B is pyridinyl; and each R⁵ is independently oxo or —(C₁₋₆)alkyl.

In an embodiment of a compound of formula (I-A), or a tautomer thereof, or a pharmaceutically acceptable salt thereof, ring B is pyridinyl; and each R⁵ is independently oxo, —F, —Cl, —NH₂, or —CH₃.

In an embodiment of a compound of formula (I-A), or a tautomer thereof, or a pharmaceutically acceptable salt thereof, ring B is pyridinyl; and each R⁵ is independently oxo, —F, —NH₂, or —CH₃.

In an embodiment of a compound of formula (I-A), or a tautomer thereof, or a pharmaceutically acceptable salt thereof, ring B is pyridinyl; and each R⁵ is independently oxo, —F, or —CH₃.

In an embodiment of a compound of formula (I-A), or a tautomer thereof, or a pharmaceutically acceptable salt thereof, ring B is pyridinyl; and each R⁵ is independently oxo or —CH₃.

In an embodiment of a compound of formula (I-A), or a tautomer thereof, or a pharmaceutically acceptable salt thereof, ring B is pyrazolyl; and each R⁵ is independently halo, oxo, —OH, —NR^{a}R^{b}, or —(C₁₋₆)alkyl.

In an embodiment of a compound of formula (I-A), or a tautomer thereof, or a pharmaceutically acceptable salt thereof, ring B is pyrazolyl; and each R⁵ is independently —(C₁₋₆)alkyl.

In an embodiment of a compound of formula (I-A), or a tautomer thereof, or a pharmaceutically acceptable salt thereof, ring B is pyrazolyl; and each R⁵ is independently —CH₃.

In an embodiment of a compound of formula (I-A), or a tautomer thereof, or a pharmaceutically acceptable salt thereof, z is 1, 2, or 3 and each R⁵ is independently —CH₃, —F, —Cl, oxo, or —NH₂.

In an embodiment of a compound of formula (I-A), or a tautomer thereof, or a pharmaceutically acceptable salt thereof,

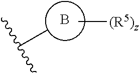

is:

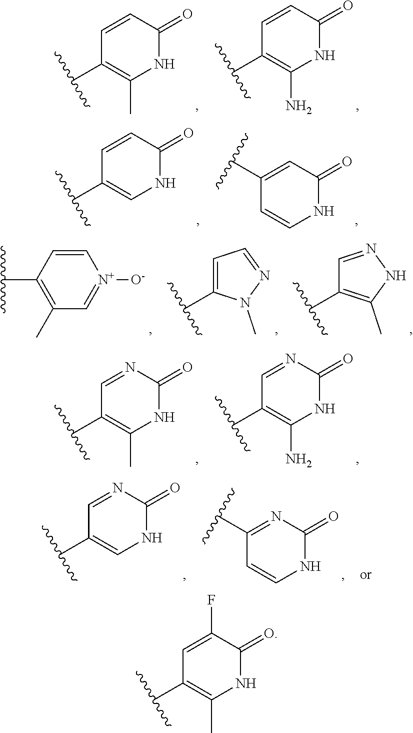

In an embodiment of a compound of formula (I-A), or a tautomer thereof, or a pharmaceutically acceptable salt thereof,

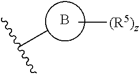

is:

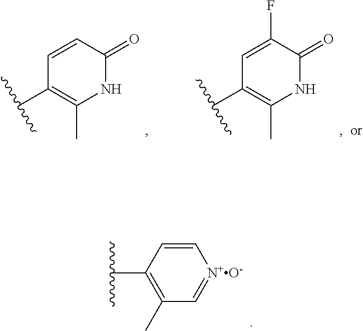

In an embodiment of a compound of formula (I-A), or a tautomer thereof, or a pharmaceutically acceptable salt thereof,

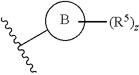

is:

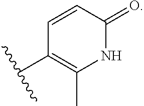 [SMILES: *c1ccc(=O)[nH]c1C]

In an embodiment of a compound of formula (I-A), or a tautomer thereof, or a pharmaceutically acceptable salt thereof, R⁶ is hydrogen.

In an embodiment of a compound of formula (I-A), or a tautomer thereof, or a pharmaceutically acceptable salt thereof, R⁶ is —(C₁₋₆)alkyl.

In an embodiment of a compound of formula (I-A), or a tautomer thereof, or a pharmaceutically acceptable salt thereof, R⁶ is —CH₃, —CH₂CH₃, or —CH(CH₃)₂.

In an embodiment of a compound of formula (I-A), or a tautomer thereof, or a pharmaceutically acceptable salt thereof, R⁶ is —CH₃.

In an embodiment of a compound of formula (I-A), or a tautomer thereof, or a pharmaceutically acceptable salt thereof, R⁶ is —NR^{a}R^{b}.

In an embodiment of a compound of formula (I-A), or a tautomer thereof, or a pharmaceutically acceptable salt thereof, R⁶ is —NH₂.

In an embodiment of a compound of formula (I-A), or a tautomer thereof, or a pharmaceutically acceptable salt thereof, each R⁷ is independently halo or —O(C₁₋₆)haloalkyl.

In an embodiment of a compound of formula (I-A), or a tautomer thereof, or a pharmaceutically acceptable salt thereof, each R⁷ is independently —F or —OCF₃.

In an embodiment of a compound of formula (I-A), or a tautomer thereof, or a pharmaceutically acceptable salt thereof, n is 1 or 2; and each R⁷ is independently —F or —OCF₃.

In an embodiment of a compound of formula (I-A), or a tautomer thereof, or a pharmaceutically acceptable salt thereof, n is 1 and R⁷ is —F.

In an embodiment of a compound of formula (I-A), or a tautomer thereof, or a pharmaceutically acceptable salt thereof, n is 1 and R⁷ is —OCF₃.

In an embodiment of a compound of formula (I-A), or a tautomer thereof, or a pharmaceutically acceptable salt thereof, n is 2 and each R⁷ is —F.

In an embodiment of a compound of formula (I-A), or a tautomer thereof, or a pharmaceutically acceptable salt thereof, R⁶ is —CH₃, —CH₂CH₃, or —CH(CH₃)₂; n is 1 or 2; and each R⁷ is independently —F or —OCF₃.

In an embodiment of a compound of formula (I-A), or a tautomer thereof, or a pharmaceutically acceptable salt thereof,

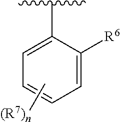

has the structure:

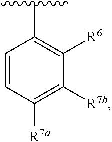

wherein each of R^{7a} and R^{7b} is independently hydrogen, halo, —(C₁₋₆)alkyl, —O—(C₁₋₆)alkyl, or —O—(C₁₋₆)-haloalkyl.

In an embodiment of a compound of formula (I-A), or a tautomer thereof, or a pharmaceutically acceptable salt thereof, each of R^{7a} and R^{7b} is independently hydrogen, halo, or —O(C₁₋₆)haloalkyl.

In an embodiment of a compound of formula (I-A), or a tautomer thereof, or a pharmaceutically acceptable salt thereof, R^{7a} is —F or —OCF₃.

In an embodiment of a compound of formula (I-A), or a tautomer thereof, or a pharmaceutically acceptable salt thereof, R^{7b} is hydrogen or —F.

In an embodiment of a compound of formula (I-A), or a tautomer thereof, or a pharmaceutically acceptable salt thereof, R^{7a} is —F or —OCF₃; and R^{7b} is hydrogen or —F.

In an embodiment of a compound of formula (I-A), or a tautomer thereof, or a pharmaceutically acceptable salt thereof, R^{7a} is —F; and R^{7b} is hydrogen.

In an embodiment of a compound of formula (I-A), or a tautomer thereof, or a pharmaceutically acceptable salt thereof,

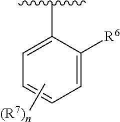

is:

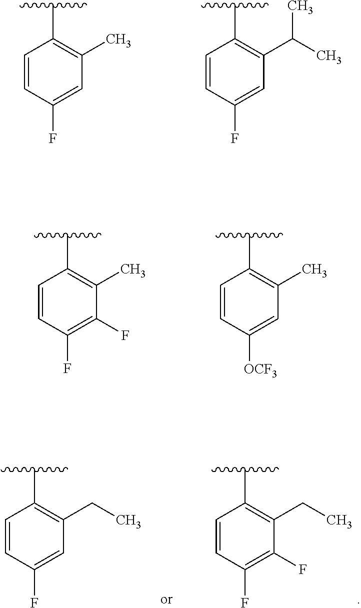

In an embodiment of a compound of formula (I-A), or a tautomer thereof, or a pharmaceutically acceptable salt thereof, each of R^{a} and R^{b} is independently hydrogen or —(C₁₋₆)alkyl.

In an embodiment of a compound of formula (I-A), or a tautomer thereof, or a pharmaceutically acceptable salt thereof:
Y is O;
each of R¹, R², and R³ is independently hydrogen, —CF₃, Cl, or cyano;
ring B is a 5- or 6-membered heteroaryl containing 1 or 2 nitrogen ring atoms;
each R⁵ is independently oxo, —F, —CH₃, or —NH₂;
R⁶ is —CH₃, —CH₂CH₃, or —CH(CH₃)₂;
each R⁷ is independently —F or —OCF₃;
z is 0, 1, 2, or 3; and
n is 1 or 2.

In another aspect, the invention relates to a compound of Formula (I-B):

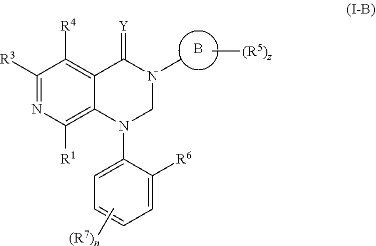

or a pharmaceutically acceptable salt or tautomer thereof,
   wherein:
   Y is O or S;
   each of R¹, R³, and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
   ring B is phenyl, or a 5- or 6-membered heterocyclyl containing 1 or 2 nitrogen ring atoms;
   each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
   R⁶ is hydrogen, —(C₁₋₆)alkyl, —O—(C₁₋₆)-alkyl, or —NR^{a}R^{b};
   each R⁷ is independently halo, —(C₁₋₆)alkyl, —O—(C₁₋₆)alkyl, or —O—(C₁₋₆)-haloalkyl;
   each of R^{a} and R^{b} is independently hydrogen, —(C₁₋₆)alkyl, or —(C₁₋₆)haloalkyl;
   R^{c} is hydrogen, —OH, —NR^{a}R^{b}, —(C₁₋₆)-alkyl, or —(C₁₋₆)-haloalkyl;
   n is 0, 1, 2, or 3;
   p is 0, 1, or 2; and
   z is 0, 1, 2, or 3.

In an embodiment of a compound of formula (I-B), or a tautomer thereof, or a pharmaceutically acceptable salt thereof,
Y is O or S;
each of R¹, R³, and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is a 5- or 6-membered heteroaryl containing 1 or 2 nitrogen ring atoms;
each R⁵ is independently halo, oxo, —OH, —NR^{a}R^{b}, or —(C₁₋₆)alkyl;
R⁶ is —(C₁₋₆)alkyl;
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
n is 0, 1, 2, or 3; and
z is 0, 1, 2, or 3.

In an embodiment of a compound of formula (I-B), or a tautomer thereof, or a pharmaceutically acceptable salt thereof, Y is O.

In an embodiment of a compound of formula (I-B), or a tautomer thereof, or a pharmaceutically acceptable salt thereof, Y is S.

In an embodiment of a compound of formula (I-B), or a tautomer thereof, or a pharmaceutically acceptable salt thereof, R¹ is hydrogen, halo, cyano, or —(C₁₋₆)-haloalkyl.

In an embodiment of a compound of formula (I-B), or a tautomer thereof, or a pharmaceutically acceptable salt thereof, R¹ is hydrogen, —CF₃, —Cl, or cyano.

In an embodiment of a compound of formula (I-B), or a tautomer thereof, or a pharmaceutically acceptable salt thereof, R¹ is hydrogen.

In an embodiment of a compound of formula (I-B), or a tautomer thereof, or a pharmaceutically acceptable salt thereof, R³ is hydrogen, halo, cyano, or —(C₁₋₆)-haloalkyl.

In an embodiment of a compound of formula (I-B), or a tautomer thereof, or a pharmaceutically acceptable salt thereof, R³ is hydrogen, —CF₃, —Cl, or cyano.

In an embodiment of a compound of formula (I-B), or a tautomer thereof, or a pharmaceutically acceptable salt thereof, R⁴ is hydrogen, halo, cyano, or —(C₁₋₆)-haloalkyl.

In an embodiment of a compound of formula (I-B), or a tautomer thereof, or a pharmaceutically acceptable salt thereof, R⁴ is hydrogen, —CF₃, —Cl, or cyano.

In an embodiment of a compound of formula (I-B), or a tautomer thereof, or a pharmaceutically acceptable salt thereof, R⁴ is hydrogen.

In an embodiment of a compound of formula (I-B), or a tautomer thereof, or a pharmaceutically acceptable salt thereof, ring B is phenyl.

In an embodiment of a compound of formula (I-B), or a tautomer thereof, or a pharmaceutically acceptable salt thereof, ring B is a 5- or 6-membered heterocyclyl containing 1 or 2 nitrogen ring atoms.

In an embodiment of a compound of formula (I-B), or a tautomer thereof, or a pharmaceutically acceptable salt thereof, ring B is a 5- or 6-membered heteroaryl containing 1 or 2 nitrogen ring atoms.

In an embodiment of a compound of formula (I-B), or a tautomer thereof, or a pharmaceutically acceptable salt thereof, ring B is a 5- or 6-membered heterocycloalkyl containing 1 or 2 nitrogen ring atoms.

In an embodiment of a compound of formula (I-B), or a tautomer thereof, or a pharmaceutically acceptable salt thereof, each R⁵ is independently halo, oxo, —OH, —NR^{a}R^{b}, or —(C₁₋₆)alkyl.

In an embodiment of a compound of formula (I-B), or a tautomer thereof, or a pharmaceutically acceptable salt thereof, ring B is a 5- or 6-membered heteroaryl containing 1 or 2 nitrogen ring atoms; and each R⁵ is independently halo, oxo, —OH, —NR^{a}R^{b}, —(C₁₋₆)alkyl, or —(C₁₋₆)haloalkyl.

In an embodiment of a compound of formula (I-B), or a tautomer thereof, or a pharmaceutically acceptable salt thereof, ring B is pyridinyl; and each R⁵ is independently halo, oxo, —OH, —NR^{a}R^{b}, or —(C₁₋₆)alkyl.

In an embodiment of a compound of formula (I-B), or a tautomer thereof, or a pharmaceutically acceptable salt thereof, ring B is pyridinyl; and each R⁵ is independently oxo or —(C₁₋₆)alkyl.

In an embodiment of a compound of formula (I-B), or a tautomer thereof, or a pharmaceutically acceptable salt thereof, ring B is pyridinyl; and each R⁵ is independently oxo, —F, —Cl, —NH₂, or —CH₃.

In an embodiment of a compound of formula (I-B), or a tautomer thereof, or a pharmaceutically acceptable salt thereof, ring B is pyridinyl; and each R⁵ is independently oxo, —F, —NH₂, or —CH₃.

In an embodiment of a compound of formula (I-B), or a tautomer thereof, or a pharmaceutically acceptable salt thereof, ring B is pyridinyl; and each R⁵ is independently oxo, —F, or —CH₃.

In an embodiment of a compound of formula (I-B), or a tautomer thereof, or a pharmaceutically acceptable salt thereof, ring B is pyridinyl; and each R⁵ is independently oxo or —CH₃.

In an embodiment of a compound of formula (I-B), or a tautomer thereof, or a pharmaceutically acceptable salt thereof, ring B is pyrazolyl; and each R⁵ is independently halo, oxo, —OH, —NR^{a}R^{b}, or —(C₁₋₆)alkyl.

In an embodiment of a compound of formula (I-B), or a tautomer thereof, or a pharmaceutically acceptable salt thereof, ring B is pyrazolyl; and each R⁵ is independently —(C₁₋₆)alkyl.

In an embodiment of a compound of formula (I-B), or a tautomer thereof, or a pharmaceutically acceptable salt thereof, ring B is pyrazolyl; and each R⁵ is independently —CH₃.

In an embodiment of a compound of formula (I-B), or a tautomer thereof, or a pharmaceutically acceptable salt thereof, z is 1, 2, or 3 and each R⁵ is independently —CH₃, —F, —C, oxo, or —NH₂.

In an embodiment of a compound of formula (I-B), or a tautomer thereof, or a pharmaceutically acceptable salt thereof,

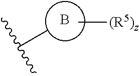

is:

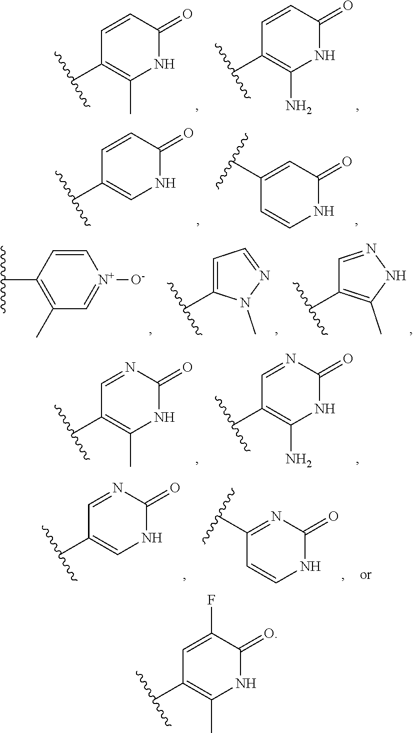

In an embodiment of a compound of formula (I-B), or a tautomer thereof, or a pharmaceutically acceptable salt thereof,

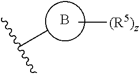

is:

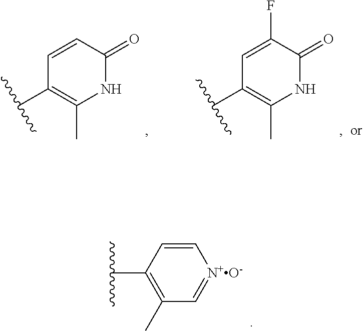

In an embodiment of a compound of formula (I-B), or a tautomer thereof, or a pharmaceutically acceptable salt thereof,

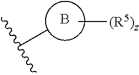

is:

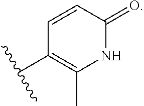 [SMILES: *c1ccc(=O)[nH]c1C]

In an embodiment of a compound of formula (I-B), or a tautomer thereof, or a pharmaceutically acceptable salt thereof, R⁶ is hydrogen.

In an embodiment of a compound of formula (I-B), or a tautomer thereof, or a pharmaceutically acceptable salt thereof, R⁶ is —(C₁₋₆)alkyl.

In an embodiment of a compound of formula (I-B), or a tautomer thereof, or a pharmaceutically acceptable salt thereof, R⁶ is —CH₃, —CH₂CH₃, or —CH(CH₃)₂.

In an embodiment of a compound of formula (I-B), or a tautomer thereof, or a pharmaceutically acceptable salt thereof, R⁶ is —CH₃.

In an embodiment of a compound of formula (I-B), or a tautomer thereof, or a pharmaceutically acceptable salt thereof, R⁶ is —NR^{a}R^{b}.

In an embodiment of a compound of formula (I-B), or a tautomer thereof, or a pharmaceutically acceptable salt thereof, R⁶ is —NH₂.

In an embodiment of a compound of formula (I-B), or a tautomer thereof, or a pharmaceutically acceptable salt thereof, each R⁷ is independently halo or —O(C₁₋₆)haloalkyl.

In an embodiment of a compound of formula (I-B), or a tautomer thereof, or a pharmaceutically acceptable salt thereof, each R⁷ is independently —F or —OCF₃.

In an embodiment of a compound of formula (I-B), or a tautomer thereof, or a pharmaceutically acceptable salt thereof, n is 1 or 2; and each R⁷ is independently —F or —OCF₃.

In an embodiment of a compound of formula (I-B), or a tautomer thereof, or a pharmaceutically acceptable salt thereof, n is 1 and R⁷ is —F.

In an embodiment of a compound of formula (I-B), or a tautomer thereof, or a pharmaceutically acceptable salt thereof, n is 1 and R⁷ is —OCF₃.

In an embodiment of a compound of formula (I-B), or a tautomer thereof, or a pharmaceutically acceptable salt thereof, n is 2 and each R⁷ is —F.

In an embodiment of a compound of formula (I-B), or a tautomer thereof, or a pharmaceutically acceptable salt thereof, R⁶ is —CH₃, —CH₂CH₃, or —CH(CH₃)₂; n is 1 or 2; and each R⁷ is independently —F or —OCF₃.

In an embodiment of a compound of formula (I-B), or a tautomer thereof, or a pharmaceutically acceptable salt thereof,

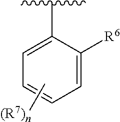

has the structure:

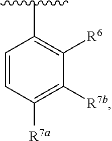

wherein each of R^{7a} and R^{7b} is independently hydrogen, halo, —(C₁₋₆)alkyl, —O—(C₁₋₆)alkyl, or —O—(C₁₋₆)-haloalkyl.

In an embodiment of a compound of formula (I-B), or a tautomer thereof, or a pharmaceutically acceptable salt thereof, each of R^{7a} and R^{7b} is independently hydrogen, halo, or —O(C₁₋₆)haloalkyl.

In an embodiment of a compound of formula (I-B), or a tautomer thereof, or a pharmaceutically acceptable salt thereof, R^{7a} is —F or —OCF₃.

In an embodiment of a compound of formula (I-B), or a tautomer thereof, or a pharmaceutically acceptable salt thereof, R^{7b} is hydrogen or —F.

In an embodiment of a compound of formula (I-B), or a tautomer thereof, or a pharmaceutically acceptable salt thereof, R^{7a} is —F or —OCF₃; and R^{7b} is hydrogen or —F.

In an embodiment of a compound of formula (I-B), or a tautomer thereof, or a pharmaceutically acceptable salt thereof, R^{7a} is —F; and R^{7b} is hydrogen.

In an embodiment of a compound of formula (I-B), or a tautomer thereof, or a pharmaceutically acceptable salt thereof,

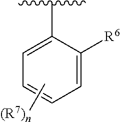

is:

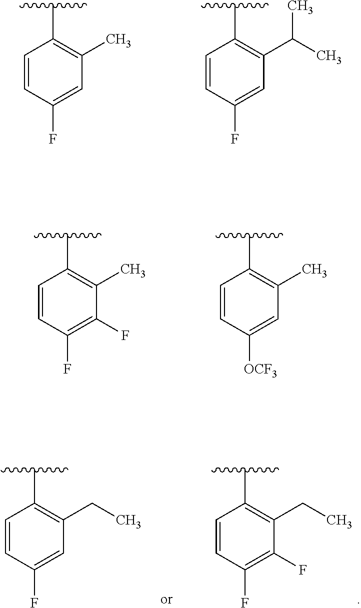

In an embodiment of a compound of formula (I-B), or a tautomer thereof, or a pharmaceutically acceptable salt thereof, each of R^{a} and R^{b} is independently hydrogen or —(C₁₋₆)alkyl.

In an embodiment of a compound of formula (I-B), or a tautomer thereof, or a pharmaceutically acceptable salt thereof:
Y is O;
each of R¹, R³, and R⁴ is independently hydrogen, —CF₃, Cl, or cyano;
ring B is a 5- or 6-membered heteroaryl containing 1 or 2 nitrogen ring atoms;
each R⁵ is independently oxo, —F, —CH₃, or —NH₂;
R⁶ is —CH₃, —CH₂CH₃, or —CH(CH₃)₂;
each R⁷ is independently —F or —OCF₃; and
n is 1 or 2.

In another aspect, the invention relates to a compound of Formula (I-C):

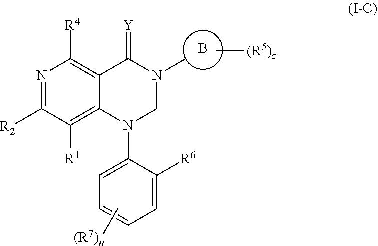

or a tautomer thereof, or a pharmaceutically acceptable salt thereof,
wherein:
   Y is O or S;
   each of R¹, R² and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
   ring B is phenyl, or a 5- or 6-membered heterocyclyl containing 1 or 2 nitrogen ring atoms;
   each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
   R⁶ is hydrogen, —(C₁₋₆)alkyl, —O—(C₁₋₆)-alkyl, or —NR^{a}R^{b};
   each R⁷ is independently halo, —(C₁₋₆)alkyl, —O—(C₁₋₆)alkyl, or —O—(C₁₋₆)-haloalkyl;
   each of R^{a} and R^{b} is independently hydrogen, —(C₁₋₆)alkyl, or —(C₁₋₆)haloalkyl;
   R^{c} is hydrogen, —OH, —NR^{a}R^{b}, —(C₁₋₆)-alkyl, or —(C₁₋₆)-haloalkyl;
   n is 0, 1, 2, or 3;
   p is 0, 1, or 2; and
   z is 0, 1, 2, or 3.

In an embodiment of a compound of formula (I-C), or a tautomer thereof, or a pharmaceutically acceptable salt thereof,
Y is O or S;
each of R¹, R², and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is a 5- or 6-membered heteroaryl containing 1 or 2 nitrogen ring atoms;
each R⁵ is independently halo, oxo, —OH, —NR^{a}R^{b}, or —(C₁₋₆)alkyl;
R⁶ is —(C₁₋₆)alkyl;
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
n is 0, 1, 2, or 3; and
z is 0, 1, 2, or 3.

In an embodiment of a compound of formula (I-C), or a tautomer thereof, or a pharmaceutically acceptable salt thereof, R¹ is hydrogen, halo, cyano, or —(C₁₋₆)-haloalkyl.

In an embodiment of a compound of formula (I-C), or a tautomer thereof, or a pharmaceutically acceptable salt thereof, R¹ is hydrogen, —CF₃, —Cl, or cyano.

In an embodiment of a compound of formula (I-C), or a tautomer thereof, or a pharmaceutically acceptable salt thereof, R¹ is hydrogen.

In an embodiment of a compound of formula (I-C), or a tautomer thereof, or a pharmaceutically acceptable salt thereof, R² is hydrogen, halo, cyano, or —(C₁₋₆)-haloalkyl.

In an embodiment of a compound of formula (I-C), or a tautomer thereof, or a pharmaceutically acceptable salt thereof, R² is hydrogen, —CF₃, —Cl, or cyano.

In an embodiment of a compound of formula (I-C), or a tautomer thereof, or a pharmaceutically acceptable salt thereof, R⁴ is hydrogen, halo, cyano, or —(C₁₋₆)-haloalkyl.

In an embodiment of a compound of formula (I-C), or a tautomer thereof, or a pharmaceutically acceptable salt thereof, R⁴ is hydrogen, —CF₃, —Cl, or cyano.

In an embodiment of a compound of formula (I-C), or a tautomer thereof, or a pharmaceutically acceptable salt thereof, R⁴ is hydrogen.

In an embodiment of a compound of formula (I-C), or a tautomer thereof, or a pharmaceutically acceptable salt thereof, ring B is phenyl.

In an embodiment of a compound of formula (I-C), or a tautomer thereof, or a pharmaceutically acceptable salt thereof, ring B is a 5- or 6-membered heterocyclyl containing 1 or 2 nitrogen ring atoms.

In an embodiment of a compound of formula (I-C), or a tautomer thereof, or a pharmaceutically acceptable salt thereof, ring B is a 5- or 6-membered heteroaryl containing 1 or 2 nitrogen ring atoms.

In an embodiment of a compound of formula (I-C), or a tautomer thereof, or a pharmaceutically acceptable salt thereof, ring B is a 5- or 6-membered heterocycloalkyl containing 1 or 2 nitrogen ring atoms.

In an embodiment of a compound of formula (I-C), or a tautomer thereof, or a pharmaceutically acceptable salt thereof, each R⁵ is independently halo, oxo, —OH, —NR^{a}R^{b}, or —(C₁₋₆)alkyl.

In an embodiment of a compound of formula (I-C), or a tautomer thereof, or a pharmaceutically acceptable salt thereof, ring B is a 5- or 6-membered heteroaryl containing 1 or 2 nitrogen ring atoms; and each R⁵ is independently halo, oxo, —OH, —NR^{a}R^{b}, —(C₁₋₆)alkyl, or —(C₁₋₆)haloalkyl.

In an embodiment of a compound of formula (I-C), or a tautomer thereof, or a pharmaceutically acceptable salt thereof, ring B is pyridinyl; and each R⁵ is independently halo, oxo, —OH, —NR^{a}R^{b}, or —(C₁₋₆)alkyl.

In an embodiment of a compound of formula (I-C), or a tautomer thereof, or a pharmaceutically acceptable salt thereof, ring B is pyridinyl; and each R⁵ is independently oxo or —(C₁₋₆)alkyl.

In an embodiment of a compound of formula (I-C), or a tautomer thereof, or a pharmaceutically acceptable salt thereof, ring B is pyridinyl; and each R⁵ is independently oxo, —F, —Cl, —NH₂, or —CH₃.

In an embodiment of a compound of formula (I-C), or a tautomer thereof, or a pharmaceutically acceptable salt thereof, ring B is pyridinyl; and each R⁵ is independently oxo, —F, —NH₂, or —CH₃.

In an embodiment of a compound of formula (I-C), or a tautomer thereof, or a pharmaceutically acceptable salt thereof, ring B is pyridinyl; and each R⁵ is independently oxo, —F, or —CH₃.

In an embodiment of a compound of formula (I-C), or a tautomer thereof, or a pharmaceutically acceptable salt thereof, ring B is pyridinyl; and each R⁵ is independently oxo or —CH₃.

In an embodiment of a compound of formula (I-C), or a tautomer thereof, or a pharmaceutically acceptable salt thereof, ring B is pyrazolyl; and each R⁵ is independently halo, oxo, —OH, —NR^{a}R^{b}, or —(C₁₋₆)alkyl.

In an embodiment of a compound of formula (I-C), or a tautomer thereof, or a pharmaceutically acceptable salt thereof, ring B is pyrazolyl; and each R⁵ is independently —(C₁₋₆)alkyl.

In an embodiment of a compound of formula (I-C), or a tautomer thereof, or a pharmaceutically acceptable salt thereof, ring B is pyrazolyl; and each R⁵ is independently —CH₃.

In an embodiment of a compound of formula (I-C), or a tautomer thereof, or a pharmaceutically acceptable salt thereof, z is 1, 2, or 3 and each R⁵ is independently —CH₃, —F, —Cl, oxo, or —NH₂.

In an embodiment of a compound of formula (I-C), or a tautomer thereof, or a pharmaceutically acceptable salt thereof,

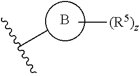

is:

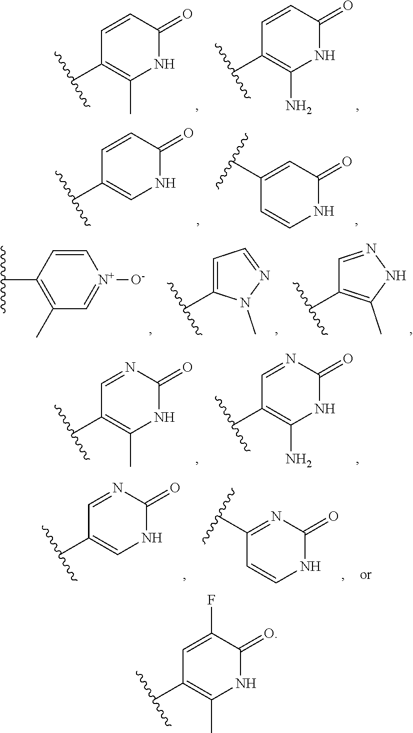

In an embodiment of a compound of formula (I-C), or a tautomer thereof, or a pharmaceutically acceptable salt thereof,

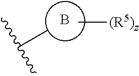

is:

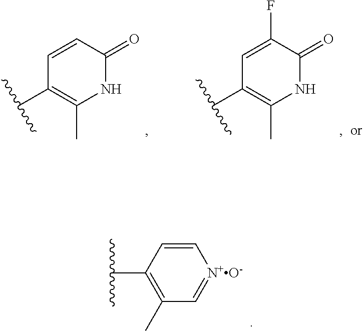

In an embodiment of a compound of formula (I-C), or a tautomer thereof, or a pharmaceutically acceptable salt thereof,

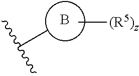

is:

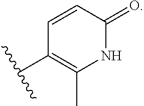 [SMILES: *c1ccc(=O)[nH]c1C]

In an embodiment of a compound of formula (I-C), or a tautomer thereof, or a pharmaceutically acceptable salt thereof, R⁶ is hydrogen.

In an embodiment of a compound of formula (I-C), or a tautomer thereof, or a pharmaceutically acceptable salt thereof, R⁶ is —(C₁₋₆)alkyl.

In an embodiment of a compound of formula (I-C), or a tautomer thereof, or a pharmaceutically acceptable salt thereof, R⁶ is —CH₃, —CH₂CH₃, or —CH(CH₃)₂.

In an embodiment of a compound of formula (I-C), or a tautomer thereof, or a pharmaceutically acceptable salt thereof, R⁶ is —CH₃.

In an embodiment of a compound of formula (I-C), or a tautomer thereof, or a pharmaceutically acceptable salt thereof, R⁶ is —NR^{a}R^{b}.

In an embodiment of a compound of formula (I-C), or a tautomer thereof, or a pharmaceutically acceptable salt thereof, R⁶ is —NH₂.

In an embodiment of a compound of formula (I-C), or a tautomer thereof, or a pharmaceutically acceptable salt thereof, each R⁷ is independently halo or —O(C₁₋₆)haloalkyl.

In an embodiment of a compound of formula (I-C), or a tautomer thereof, or a pharmaceutically acceptable salt thereof, each R⁷ is independently —F or —OCF₃.

In an embodiment of a compound of formula (I-C), or a tautomer thereof, or a pharmaceutically acceptable salt thereof, n is 1 or 2; and each R⁷ is independently —F or —OCF₃.

In an embodiment of a compound of formula (I-C), or a tautomer thereof, or a pharmaceutically acceptable salt thereof, n is 1 and R⁷ is —F.

In an embodiment of a compound of formula (I-C), or a tautomer thereof, or a pharmaceutically acceptable salt thereof, n is 1 and R⁷ is —OCF₃.

In an embodiment of a compound of formula (I-C), or a tautomer thereof, or a pharmaceutically acceptable salt thereof, n is 2 and each R⁷ is —F.

In an embodiment of a compound of formula (I-C), or a tautomer thereof, or a pharmaceutically acceptable salt thereof, R⁶ is —CH₃, —CH₂CH₃, or —CH(CH₃)₂; n is 1 or 2; and each R⁷ is independently —F or —OCF₃.

In an embodiment of a compound of formula (I-C), or a tautomer thereof, or a pharmaceutically acceptable salt thereof,

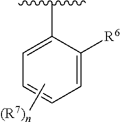

has the structure:

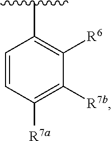

wherein each of R^{7a} and R^{7b} is independently hydrogen, halo, —(C₁₋₆)alkyl, —O—(C₁₋₆)alkyl, or —O—(C₁₋₆)-haloalkyl.

In an embodiment of a compound of formula (I-C), or a tautomer thereof, or a pharmaceutically acceptable salt thereof, each of R^{7a} and R^{7b} is independently hydrogen, halo, or —O(C₁₋₆)haloalkyl.

In an embodiment of a compound of formula (I-C), or a tautomer thereof, or a pharmaceutically acceptable salt thereof, R^{7a} is —F or —OCF₃.

In an embodiment of a compound of formula (I-C), or a tautomer thereof, or a pharmaceutically acceptable salt thereof, R^{7b} is hydrogen or —F.

In an embodiment of a compound of formula (I-C), or a tautomer thereof, or a pharmaceutically acceptable salt thereof, R^{7a} is —F or —OCF₃; and R^{7b} is hydrogen or —F.

In an embodiment of a compound of formula (I-C), or a tautomer thereof, or a pharmaceutically acceptable salt thereof, R^{7a} is —F; and R^{7b} is hydrogen.

In an embodiment of a compound of formula (I-C), or a tautomer thereof, or a pharmaceutically acceptable salt thereof,

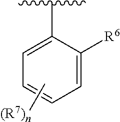

is:

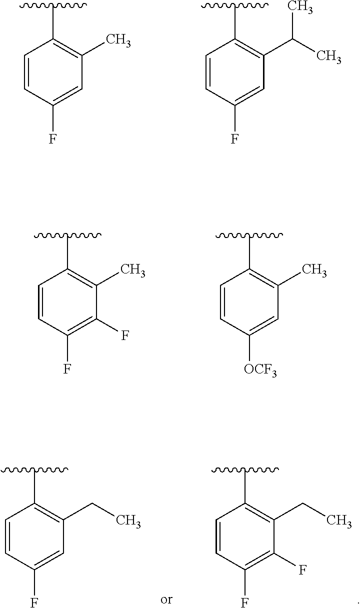

In an embodiment of a compound of formula (I-C), or a tautomer thereof, or a pharmaceutically acceptable salt thereof, each of R^{a} and R^{b} is independently hydrogen or —(C₁₋₆)alkyl.

In an embodiment of a compound of formula (I-C), or a tautomer thereof, or a pharmaceutically acceptable salt thereof:
Y is O;
each of R¹, R², and R³ is independently hydrogen, —CF₃, Cl, or cyano;
ring B is a 5- or 6-membered heteroaryl containing 1 or 2 nitrogen ring atoms;
each R⁵ is independently oxo, —F, —CH₃, or —NH₂;
R⁶ is —CH₃, —CH₂CH₃, or —CH(CH₃)₂;
each R⁷ is independently —F or —OCF₃;
z is 0, 1, 2, or 3; and
n is 1 or 2.

In another aspect, the invention relates to a compound of formula (I-D):

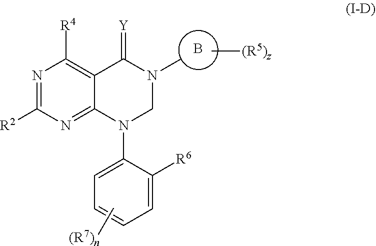

or a pharmaceutically acceptable salt or tautomer thereof,
wherein:
   Y is O or S;
   each of R² and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
   ring B is phenyl, or a 5- or 6-membered heterocyclyl containing 1 or 2 nitrogen ring atoms;
   each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
   R⁶ is hydrogen, —(C₁₋₆)alkyl, —O—(C₁₋₆)-alkyl, or —NR^{a}R^{b};
   each R⁷ is independently halo, —(C₁₋₆)alkyl, —O—(C₁₋₆)alkyl, or —O—(C₁₋₆)-haloalkyl;
   each of R^{a} and R^{b} is independently hydrogen, —(C₁₋₆)alkyl, or —(C₁₋₆)haloalkyl;
   R^{c} is hydrogen, —OH, —NR^{a}R^{b}, —(C₁₋₆)-alkyl, or —(C₁₋₆)-haloalkyl;
   n is 0, 1, 2, or 3;
   p is 0, 1, or 2; and
   z is 0, 1, 2, or 3.

In an embodiment of a compound of formula (I-D), or a tautomer thereof, or a pharmaceutically acceptable salt thereof,
Y is O or S;
each of R² and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is a 5- or 6-membered heteroaryl containing 1 or 2 nitrogen ring atoms;
each R⁵ is independently halo, oxo, —OH, —NR^{a}R^{b}, or —(C₁₋₆)alkyl;
R⁶ is —(C₁₋₆)alkyl;
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
n is 0, 1, 2, or 3; and
z is 0, 1, 2, or 3.

In an embodiment of a compound of formula (I-D), or a tautomer thereof, or a pharmaceutically acceptable salt thereof, Y is O.

In an embodiment of a compound of formula (I-D), or a tautomer thereof, or a pharmaceutically acceptable salt thereof, Y is S.

In an embodiment of a compound of formula (I-D), or a tautomer thereof, or a pharmaceutically acceptable salt thereof, R² is hydrogen, halo, cyano, or —(C₁₋₆)-haloalkyl.

In an embodiment of a compound of formula (I-D), or a tautomer thereof, or a pharmaceutically acceptable salt thereof, R² is hydrogen, —CF₃, —Cl, or cyano.

In an embodiment of a compound of formula (I-D), or a tautomer thereof, or a pharmaceutically acceptable salt thereof, R² is hydrogen, —CF₃, —Cl, or cyano.

In an embodiment of a compound of formula (I-D), or a tautomer thereof, or a pharmaceutically acceptable salt thereof, R⁴ is hydrogen, halo, cyano, or —(C₁₋₆)-haloalkyl.

In an embodiment of a compound of formula (I-C), or a tautomer thereof, or a pharmaceutically acceptable salt thereof, R⁴ is hydrogen, —CF₃, —Cl, or cyano.

In an embodiment of a compound of formula (I-D), or a tautomer thereof, or a pharmaceutically acceptable salt thereof, R⁴ is hydrogen.

In an embodiment of a compound of formula (I-D), or a tautomer thereof, or a pharmaceutically acceptable salt thereof, ring B is phenyl.

In an embodiment of a compound of formula (I-D), or a tautomer thereof, or a pharmaceutically acceptable salt thereof, ring B is a 5- or 6-membered heterocyclyl containing 1 or 2 nitrogen ring atoms.

In an embodiment of a compound of formula (I-D), or a tautomer thereof, or a pharmaceutically acceptable salt thereof, ring B is a 5- or 6-membered heteroaryl containing 1 or 2 nitrogen ring atoms.

In an embodiment of a compound of formula (I-D), or a tautomer thereof, or a pharmaceutically acceptable salt thereof, ring B is a 5- or 6-membered heterocycloalkyl containing 1 or 2 nitrogen ring atoms.

In an embodiment of a compound of formula (I-D), or a tautomer thereof, or a pharmaceutically acceptable salt thereof, each R⁵ is independently halo, oxo, —OH, —NR^{a}R^{b}, or —(C₁₋₆)alkyl.

In an embodiment of a compound of formula (I-D), or a tautomer thereof, or a pharmaceutically acceptable salt thereof, ring B is a 5- or 6-membered heteroaryl containing 1 or 2 nitrogen ring atoms; and each R⁵ is independently halo, oxo, —OH, —NR^{a}R^{b}, —(C₁₋₆)alkyl, or —(C₁₋₆)haloalkyl.

In an embodiment of a compound of formula (I-D), or a tautomer thereof, or a pharmaceutically acceptable salt thereof, ring B is pyridinyl; and each R⁵ is independently halo, oxo, —OH, —NR^{a}R^{b}, or —(C₁₋₆)alkyl.

In an embodiment of a compound of formula (I-D), or a tautomer thereof, or a pharmaceutically acceptable salt thereof, ring B is pyridinyl; and each R⁵ is independently oxo or —(C₁₋₆)alkyl.

In an embodiment of a compound of formula (I-D), or a tautomer thereof, or a pharmaceutically acceptable salt thereof, ring B is pyridinyl; and each R⁵ is independently oxo, —F, —Cl, —NH₂, or —CH₃.

In an embodiment of a compound of formula (I-D), or a tautomer thereof, or a pharmaceutically acceptable salt thereof, ring B is pyridinyl; and each R⁵ is independently oxo, —F, —NH₂, or —CH₃.

In an embodiment of a compound of formula (I-D), or a tautomer thereof, or a pharmaceutically acceptable salt thereof, ring B is pyridinyl; and each R⁵ is independently oxo, —F, or —CH₃.

In an embodiment of a compound of formula (I-D), or a tautomer thereof, or a pharmaceutically acceptable salt thereof, ring B is pyridinyl; and each R⁵ is independently oxo or —CH₃.

In an embodiment of a compound of formula (I-D), or a tautomer thereof, or a pharmaceutically acceptable salt thereof, ring B is pyrazolyl; and each R⁵ is independently halo, oxo, —OH, —NR^{a}R^{b}, or —(C₁₋₆)alkyl.

In an embodiment of a compound of formula (I-D), or a tautomer thereof, or a pharmaceutically acceptable salt thereof, ring B is pyrazolyl; and each R⁵ is independently —(C₁₋₆)alkyl.

In an embodiment of a compound of formula (I-D), or a tautomer thereof, or a pharmaceutically acceptable salt thereof, ring B is pyrazolyl; and each R⁵ is independently —CH₃.

In an embodiment of a compound of formula (I-D), or a tautomer thereof, or a pharmaceutically acceptable salt thereof, z is 1, 2, or 3 and each R⁵ is independently —CH₃, —F, —Cl, oxo, or —NH₂.

In an embodiment of a compound of formula (I-D), or a tautomer thereof, or a pharmaceutically acceptable salt thereof,

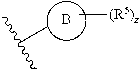

is:

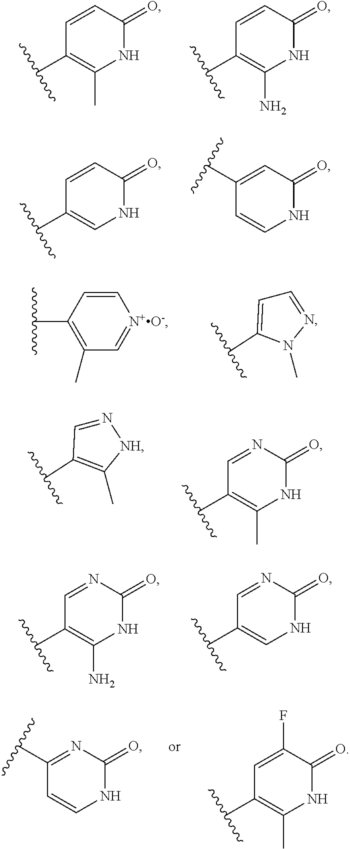

In an embodiment of a compound of formula (I-D), or a tautomer thereof, or a pharmaceutically acceptable salt thereof,

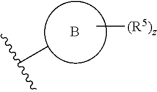

is:

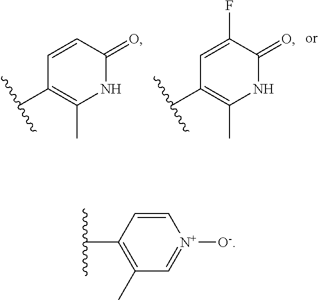

In an embodiment of a compound of formula (I-D), or a tautomer thereof, or a pharmaceutically acceptable salt thereof,

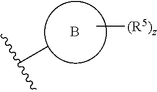

is:

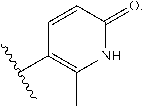 [SMILES: *c1ccc(=O)[nH]c1C]

In an embodiment of a compound of formula (I-D), or a tautomer thereof, or a pharmaceutically acceptable salt thereof, R⁶ is hydrogen.

In an embodiment of a compound of formula (I-D), or a tautomer thereof, or a pharmaceutically acceptable salt thereof, R⁶ is —(C₁₋₆)alkyl.

In an embodiment of a compound of formula (I-D), or a tautomer thereof, or a pharmaceutically acceptable salt thereof, R⁶ is —CH₃, —CH₂CH₃, or —CH(CH₃)₂.

In an embodiment of a compound of formula (I-D), or a tautomer thereof, or a pharmaceutically acceptable salt thereof, R⁶ is —CH₃.

In an embodiment of a compound of formula (I-D), or a tautomer thereof, or a pharmaceutically acceptable salt thereof, R⁶ is —NR^{a}R^{b}.

In an embodiment of a compound of formula (I-D), or a tautomer thereof, or a pharmaceutically acceptable salt thereof, R⁶ is —NH₂.

In an embodiment of a compound of formula (I-D), or a tautomer thereof, or a pharmaceutically acceptable salt thereof, each R⁷ is independently halo or —O(C₁₋₆)haloalkyl.

In an embodiment of a compound of formula (I-D), or a tautomer thereof, or a pharmaceutically acceptable salt thereof, each R⁷ is independently —F or —OCF₃.

In an embodiment of a compound of formula (I-D), or a tautomer thereof, or a pharmaceutically acceptable salt thereof, n is 1 or 2; and each R⁷ is independently —F or —OCF₃.

In an embodiment of a compound of formula (I-D), or a tautomer thereof, or a pharmaceutically acceptable salt thereof, n is 1 and R⁷ is —F.

In an embodiment of a compound of formula (I-D), or a tautomer thereof, or a pharmaceutically acceptable salt thereof, n is 1 and R⁷ is —OCF₃.

In an embodiment of a compound of formula (I-D), or a tautomer thereof, or a pharmaceutically acceptable salt thereof, n is 2 and each R⁷ is —F.

In an embodiment of a compound of formula (I-D), or a tautomer thereof, or a pharmaceutically acceptable salt thereof, R⁶ is —CH₃, —CH₂CH₃, or —CH(CH₃)₂; n is 1 or 2; and each R⁷ is independently —F or —OCF₃.

In an embodiment of a compound of formula (I-D), or a tautomer thereof, or a pharmaceutically acceptable salt thereof,

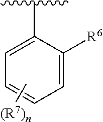

has the structure:

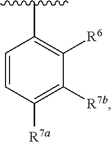

wherein each of R^{7a} and R^{7b} is independently hydrogen, halo, —(C₁₋₆)alkyl, —O—(C₁₋₆)alkyl, or —O—(C₁₋₆)-haloalkyl.

In an embodiment of a compound of formula (I-D), or a tautomer thereof, or a pharmaceutically acceptable salt thereof, each of R^{7a} and R^{7b} is independently hydrogen, halo, or —O(C₁₋₆)haloalkyl.

In an embodiment of a compound of formula (I-D), or a tautomer thereof, or a pharmaceutically acceptable salt thereof, R^{7a} is —F or —OCF₃.

In an embodiment of a compound of formula (I-D), or a tautomer thereof, or a pharmaceutically acceptable salt thereof, R^{7b} is hydrogen or —F.

In an embodiment of a compound of formula (I-D), or a tautomer thereof, or a pharmaceutically acceptable salt thereof, R^{7a} is —F or —OCF₃; and R^{7b} is hydrogen or —F.

In an embodiment of a compound of formula (I-D), or a tautomer thereof, or a pharmaceutically acceptable salt thereof, R^{7a} is —F; and R^{7b} is hydrogen.

In an embodiment of a compound of formula (I-D), or a tautomer thereof, or a pharmaceutically acceptable salt thereof,

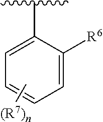

is:

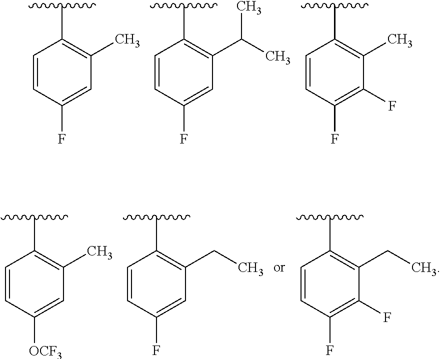

In an embodiment of a compound of formula (I-D), or a tautomer thereof, or a pharmaceutically acceptable salt thereof, each of R^{a} and R^{b} is independently hydrogen or —(C₁₋₆)alkyl.

In an embodiment of a compound of formula (I-D), or a tautomer thereof, or a pharmaceutically acceptable salt thereof:
Y is O;
each of R² and R⁴ is independently hydrogen, —CF₃, Cl, or cyano;
ring B is a 5- or 6-membered heteroaryl containing 1 or 2 nitrogen ring atoms;
each R⁵ is independently oxo, —F, —CH₃, or —NH₂;
R⁶ is —CH₃, —CH₂CH₃, or —CH(CH₃)₂;
each R⁷ is independently —F or —OCF₃;
z is 0, 1, 2, or 3; and
n is 1 or 2.

In another aspect, the invention relates to a compound of formula (I-E):

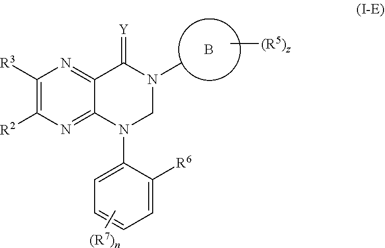

or a tautomer thereof, or a pharmaceutically acceptable salt thereof,
wherein:
   Y is O or S;
   each of R² and R³ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
   ring B is phenyl, or a 5- or 6-membered heterocyclyl containing 1 or 2 nitrogen ring atoms;
   each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
   R⁶ is hydrogen, —(C₁₋₆)alkyl, —O—(C₁₋₆)-alkyl, or —NR^{a}R^{b};
   each R⁷ is independently halo, —(C₁₋₆)alkyl, —O—(C₁₋₆)alkyl, or —O—(C₁₋₆)-haloalkyl;
   each of R^{a} and R^{b} is independently hydrogen, —(C₁₋₆)alkyl, or —(C₁₋₆)haloalkyl;
   R^{c} is hydrogen, —OH, —NR^{a}R^{b}, —(C₁₋₆)-alkyl, or —(C₁₋₆)-haloalkyl;
   n is 0, 1, 2, or 3;
   p is 0, 1, or 2; and
   z is 0, 1, 2, or 3.

In an embodiment of a compound of formula (I-D), or a tautomer thereof, or a pharmaceutically acceptable salt thereof,
Y is O or S;
each of R² and R³ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is a 5- or 6-membered heteroaryl containing 1 or 2 nitrogen ring atoms;
each R⁵ is independently halo, oxo, —OH, —NR^{a}R^{b}, or —(C₁₋₆)alkyl;
R⁶ is —(C₁₋₆)alkyl;
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
n is 0, 1, 2, or 3; and
z is 0, 1, 2, or 3.

In an embodiment of a compound of formula (I-E), or a tautomer thereof, or a pharmaceutically acceptable salt thereof, Y is O.

In an embodiment of a compound of formula (I-E), or a tautomer thereof, or a pharmaceutically acceptable salt thereof, Y is S.

In an embodiment of a compound of formula (I-E), or a tautomer thereof, or a pharmaceutically acceptable salt thereof, R² is hydrogen, halo, cyano, or —(C₁₋₆)-haloalkyl.

In an embodiment of a compound of formula (I-E), or a tautomer thereof, or a pharmaceutically acceptable salt thereof, R² is hydrogen, —CF₃, —Cl, or cyano.

In an embodiment of a compound of formula (I-E), or a tautomer thereof, or a pharmaceutically acceptable salt thereof, R³ is hydrogen, halo, cyano, or —(C₁₋₆)-haloalkyl.

In an embodiment of a compound of formula (I-E), or a tautomer thereof, or a pharmaceutically acceptable salt thereof, R³ is hydrogen, —CF₃, —Cl, or cyano.

In an embodiment of a compound of formula (I-E), or a tautomer thereof, or a pharmaceutically acceptable salt thereof, ring B is phenyl.

In an embodiment of a compound of formula (I-E), or a tautomer thereof, or a pharmaceutically acceptable salt thereof, ring B is a 5- or 6-membered heterocyclyl containing 1 or 2 nitrogen ring atoms.

In an embodiment of a compound of formula (I-E), or a tautomer thereof, or a pharmaceutically acceptable salt thereof, ring B is a 5- or 6-membered heteroaryl containing 1 or 2 nitrogen ring atoms.

In an embodiment of a compound of formula (I-E), or a tautomer thereof, or a pharmaceutically acceptable salt thereof, ring B is a 5- or 6-membered heterocycloalkyl containing 1 or 2 nitrogen ring atoms.

In an embodiment of a compound of formula (I-E), or a tautomer thereof, or a pharmaceutically acceptable salt thereof, each R⁵ is independently halo, oxo, —OH, —NR^{a}R^{b}, or —(C₁₋₆)alkyl.

In an embodiment of a compound of formula (I-E), or a tautomer thereof, or a pharmaceutically acceptable salt thereof, ring B is a 5- or 6-membered heteroaryl containing 1 or 2 nitrogen ring atoms; and each R⁵ is independently halo, oxo, —OH, —NR^{a}R^{b}, —(C₁₋₆)alkyl, or —(C₁₋₆)haloalkyl.

In an embodiment of a compound of formula (I-E), or a tautomer thereof, or a pharmaceutically acceptable salt thereof, ring B is pyridinyl; and each R⁵ is independently halo, oxo, —OH, —NR^{a}R^{b}, or —(C₁₋₆)alkyl.

In an embodiment of a compound of formula (I-E), or a tautomer thereof, or a pharmaceutically acceptable salt thereof, ring B is pyridinyl; and each R⁵ is independently oxo or —(C₁₋₆)alkyl.

In an embodiment of a compound of formula (I-E), or a tautomer thereof, or a pharmaceutically acceptable salt thereof, ring B is pyridinyl; and each R⁵ is independently oxo, —F, —Cl, —NH₂, or —CH₃.

In an embodiment of a compound of formula (I-E), or a tautomer thereof, or a pharmaceutically acceptable salt thereof, ring B is pyridinyl; and each R⁵ is independently oxo, —F, —NH₂, or —CH₃.

In an embodiment of a compound of formula (I-E), or a tautomer thereof, or a pharmaceutically acceptable salt thereof, ring B is pyridinyl; and each R⁵ is independently oxo, —F, or —CH₃.

In an embodiment of a compound of formula (I-E), or a tautomer thereof, or a pharmaceutically acceptable salt thereof, ring B is pyridinyl; and each R⁵ is independently oxo or —CH₃.

In an embodiment of a compound of formula (I-E), or a tautomer thereof, or a pharmaceutically acceptable salt thereof, ring B is pyrazolyl; and each R⁵ is independently halo, oxo, —OH, —NR^{a}R^{b}, or —(C₁₋₆)alkyl.

In an embodiment of a compound of formula (I-E), or a tautomer thereof, or a pharmaceutically acceptable salt thereof, ring B is pyrazolyl; and each R⁵ is independently —(C₁₋₆)alkyl.

In an embodiment of a compound of formula (I-E), or a tautomer thereof, or a pharmaceutically acceptable salt thereof, ring B is pyrazolyl; and each R⁵ is independently —CH₃.

In an embodiment of a compound of formula (I-E), or a tautomer thereof, or a pharmaceutically acceptable salt thereof, z is 1, 2, or 3 and each R⁵ is independently —CH₃, —F, —Cl, oxo, or —NH₂.

In an embodiment of a compound of formula (I-E), or a tautomer thereof, or a pharmaceutically acceptable salt thereof,

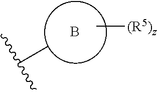

is:

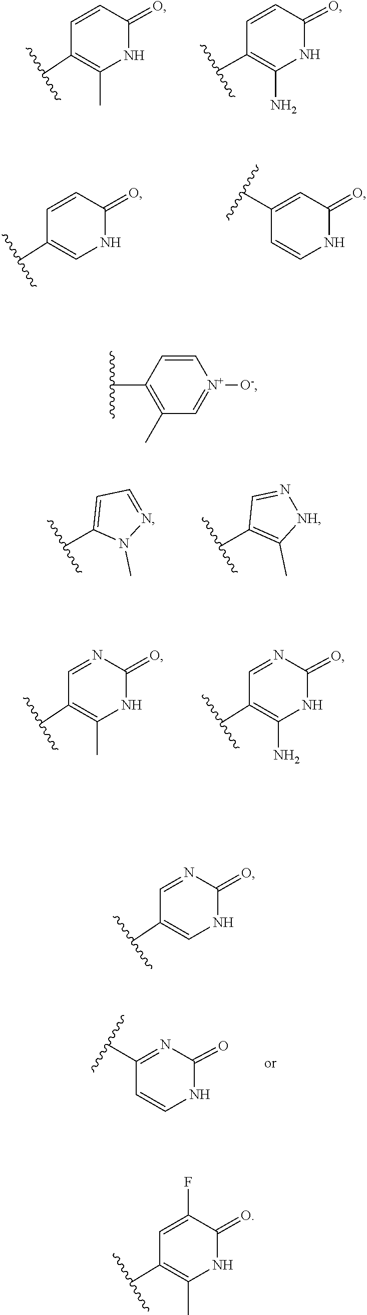

In an embodiment of a compound of formula (I-E), or a tautomer thereof, or a pharmaceutically acceptable salt thereof,

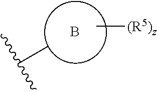

is:

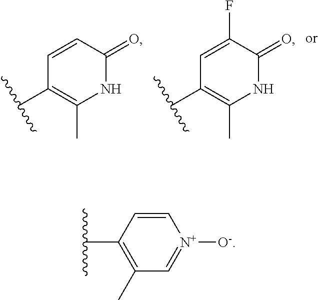

In an embodiment of a compound of formula (I-E), or a tautomer thereof, or a pharmaceutically acceptable salt thereof,

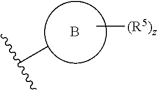

is:

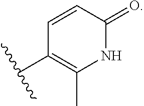 [SMILES: *c1ccc(=O)[nH]c1C]

In an embodiment of a compound of formula (I-E), or a tautomer thereof, or a pharmaceutically acceptable salt thereof, R¹ is hydrogen.

In an embodiment of a compound of formula (I-E), or a tautomer thereof, or a pharmaceutically acceptable salt thereof, R⁶ is —(C₁₋₆)alkyl.

In an embodiment of a compound of formula (I-E), or a tautomer thereof, or a pharmaceutically acceptable salt thereof, R⁶ is —OH₃, —CH₂CH₃, or —CH(CH₃)₂.

In an embodiment of a compound of formula (I-E), or a tautomer thereof, or a pharmaceutically acceptable salt thereof, R⁶ is —CH₃.

In an embodiment of a compound of formula (I-E), or a tautomer thereof, or a pharmaceutically acceptable salt thereof, R⁶ is —NR^{a}R^{b}.

In an embodiment of a compound of formula (I-E), or a tautomer thereof, or a pharmaceutically acceptable salt thereof, R⁶ is —NH₂.

In an embodiment of a compound of formula (I-E), or a tautomer thereof, or a pharmaceutically acceptable salt thereof, each R⁷ is independently halo or —O(C₁)haloalkyl.

In an embodiment of a compound of formula (I-E), or a tautomer thereof, or a pharmaceutically acceptable salt thereof, each R⁷ is independently —F or —OCF₃.

In an embodiment of a compound of formula (I-E), or a tautomer thereof, or a pharmaceutically acceptable salt thereof, n is 1 or 2; and each R⁷ is independently —F or —OCF₃.

In an embodiment of a compound of formula (I-E), or a tautomer thereof, or a pharmaceutically acceptable salt thereof, n is 1 and R⁷ is —F.

In an embodiment of a compound of formula (I-E), or a tautomer thereof, or a pharmaceutically acceptable salt thereof, n is 1 and R⁷ is —OCF₃.

In an embodiment of a compound of formula (I-E), or a tautomer thereof, or a pharmaceutically acceptable salt thereof, n is 2 and each R⁷ is —F.

In an embodiment of a compound of formula (I-E), or a tautomer thereof, or a pharmaceutically acceptable salt thereof, R⁶ is —CH₃, —CH₂CH₃, or —CH(CH₃)₂; n is 1 or 2; and each R⁷ is independently —F or —OCF₃.

In an embodiment of a compound of formula (I-E), or a tautomer thereof, or a pharmaceutically acceptable salt thereof,

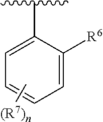

has the structure:

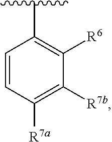

wherein each of R^{7a} and R^{7b} is independently hydrogen, halo, —(C₁₋₆)alkyl, —O—(C₁₋₆)alkyl, or —O—(C₁₋₆)-haloalkyl.

In an embodiment of a compound of formula (I-E), or a tautomer thereof, or a pharmaceutically acceptable salt thereof, each of R^{7a} and R^{7b} is independently hydrogen, halo, or —O(C₁₋₆)haloalkyl.

In an embodiment of a compound of formula (I-E), or a tautomer thereof, or a pharmaceutically acceptable salt thereof, R^{7a} is —F or —OCF₃.

In an embodiment of a compound of formula (I-E), or a tautomer thereof, or a pharmaceutically acceptable salt thereof, R^{7b} is hydrogen or —F.

In an embodiment of a compound of formula (I-E), or a tautomer thereof, or a pharmaceutically acceptable salt thereof, R^{7a} is —F or —OCF₃; and R^{7b} is hydrogen or —F.

In an embodiment of a compound of formula (I-E), or a tautomer thereof, or a pharmaceutically acceptable salt thereof, R^{7a} is —F; and R^{7b} is hydrogen.

In an embodiment of a compound of formula (I-E), or a tautomer thereof, or a pharmaceutically acceptable salt thereof,

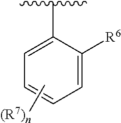

is:

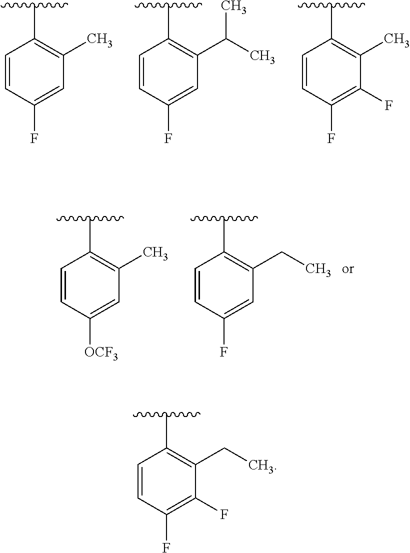

In an embodiment of a compound of formula (I-E), or a tautomer thereof, or a pharmaceutically acceptable salt thereof, each of R^{a} and R^{b} is independently hydrogen or —(C₁₋₆)alkyl.

In an embodiment of a compound of formula (I-E), or a tautomer thereof, or a pharmaceutically acceptable salt thereof:
Y is O;
each of R² and R³ is independently hydrogen, —CF₃, Cl, or cyano;
ring B is a 5- or 6-membered heteroaryl containing 1 or 2 nitrogen ring atoms;
each R⁵ is independently oxo, —F, —CH₃, or —NH₂;
R⁶ is —CH₃, —CH₂CH₃, or —CH(CH₃)₂;
each R⁷ is independently —F or —OCF₃;
z is 0, 1, 2, or 3; and
n is 1 or 2.

In another aspect, the invention relates to a compound which is selected from:

| Name | Structure |
|---|---|
| 1-(4-Fluoro-2-methylphenyl)-3-(2-methyl-6- oxo-1,6-dihydropyridin-3-yl)-7- (trifluoromethyl)-2,3-dihydropyrido[3,2- d]pyrimidin-4(1H)-one | 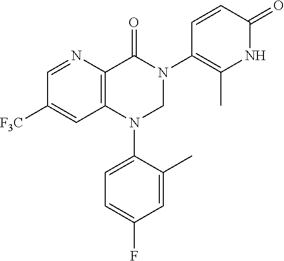 [SMILES: Cc1cc(F)ccc1N1CN(c2ccc(=O)[nH]c2C)C(=O)c2ncc(C(F)(F)F)cc21] |
| 1-(4-Fluoro-2-methylphenyl)-3-(2-methyl-6- oxo-1,6-dihydropyridin-3-yl)-7- (trifluoromethyl)-2,3-dihydropyrimido[4,5- d]pyrimidin-4(1H)-one | 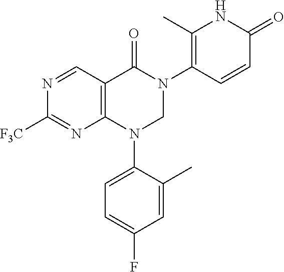 [SMILES: Cc1cc(F)ccc1N1CN(c2ccc(=O)[nH]c2C)C(=O)c2cnc(C(F)(F)F)nc21] |
| 6-Chloro-1-(4-fluoro-2-isopropylphenyl)-3- (2-methyl-6-oxo-1,6-dihydropyridin-3-yl)- 2,3-dihydropyrido[3,4-d]pyrimidin-4(1H)- one | 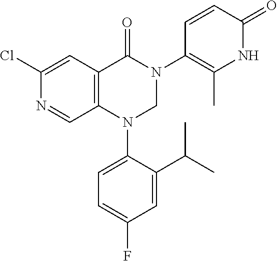 [SMILES: Cc1[nH]c(=O)ccc1N1CN(c2ccc(F)cc2C(C)C)c2cnc(Cl)cc2C1=O] |
| 1-(4-Fluoro-2-methylphenyl)-3-(2-methyl-6- oxo-1,6-dihydropyridin-3-yl)-7- (trifluoromethyl)-2,3-dihydropyrido[4,3- d]pyrimidin-4(1H)-one | 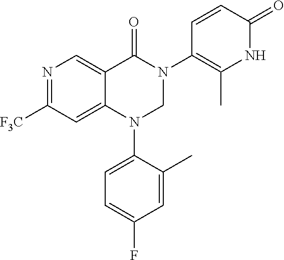 [SMILES: Cc1cc(F)ccc1N1CN(c2ccc(=O)[nH]c2C)C(=O)c2cnc(C(F)(F)F)cc21] |
| 1-(4-Fluoro-2-isopropylphenyl)-3-(2-methyl- 6-oxo-1,6-dihydropyridin-3-yl)-6- (trifluoromethyl)-2,3-dihydropyrido[3,4- d]pyrimidin-4(1H)-one | 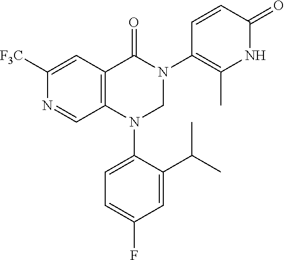 [SMILES: Cc1[nH]c(=O)ccc1N1CN(c2ccc(F)cc2C(C)C)c2cnc(C(F)(F)F)cc2C1=O] |
| 1-(4-Fluoro-2-methylphenyl)-3-(2-methyl-6- oxo-1,6-dihydropyridin-3-yl)-6- (trifluoromethyl)-2,3-dihydropyrido[3,4- d]pyrimidin-4(1H)-one | 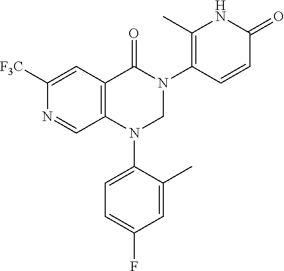 [SMILES: Cc1cc(F)ccc1N1CN(c2ccc(=O)[nH]c2C)C(=O)c2cc(C(F)(F)F)ncc21] |
| 1-(4-Fluoro-2-methylphenyl)-3-(2-methyl-6- oxo-1,6-dihydropyridin-3-yl)-7- (trifluoromethyl)-2,3-dihydropteridin-4(1H)- one | 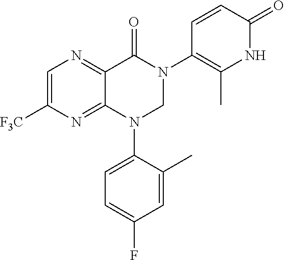 [SMILES: Cc1cc(F)ccc1N1CN(c2ccc(=O)[nH]c2C)C(=O)c2ncc(C(F)(F)F)nc21] |
| 1-(4-Fluoro-2-methylphenyl)-3-(2-methyl-6- oxo-1,6-dihydropyridin-3-yl)-6- (trifluoromethyl)-2,3-dihydropteridin-4(1H)- one | 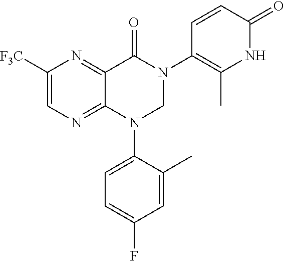 [SMILES: Cc1cc(F)ccc1N1CN(c2ccc(=O)[nH]c2C)C(=O)c2nc(C(F)(F)F)cnc21] |
| 6-Chloro-1-(4-fluoro-2-isopropylphenyl)-3- (2-methyl-6-oxo-1,6-dihydropyridin-3-yl)- 2,3-dihydropyrido[3,2-d]pyrimidin-4(1H)- one | 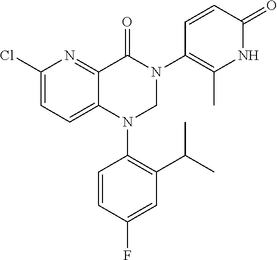 [SMILES: Cc1[nH]c(=O)ccc1N1CN(c2ccc(F)cc2C(C)C)c2ccc(Cl)nc2C1=O] |
| 1-(2-Ethyl-3,4-difluorophenyl)-3-(2-methyl- 6-oxo-1,6-dihydropyridin-3-yl)-6- (trifluoromethyl)-2,3-dihydropyrido[3,4- d]pyrimidin-4(1H)-one | 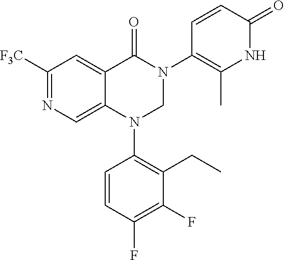 [SMILES: CCc1c(N2CN(c3ccc(=O)[nH]c3C)C(=O)c3cc(C(F)(F)F)ncc32)ccc(F)c1F] |
| 3-(5-Fluoro-2-methyl-6-oxo-1,6- dihydropyridin-3-yl)-1-(4-fluoro-2- methylphenyl)-6-(trifluoromethyl)-2,3- dihydropyrido[3,4-d]pyrimidin-4(1H)-one | 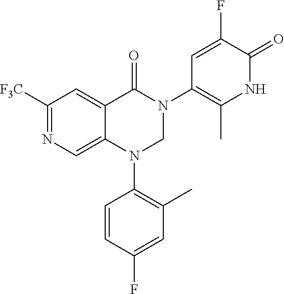 [SMILES: Cc1cc(F)ccc1N1CN(c2cc(F)c(=O)[nH]c2C)C(=O)c2cc(C(F)(F)F)ncc21] |
| 1-(4-Fluoro-2-methylphenyl)-3-(2-methyl-6- oxo-1,6-dihydropyridin-3-yl)-2,3- dihydropyrido[3,4-d]pyrimidin-4(1H)-one | 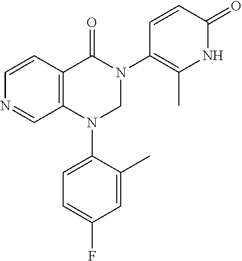 [SMILES: Cc1cc(F)ccc1N1CN(c2ccc(=O)[nH]c2C)C(=O)c2ccncc21] |
| 1-(2-Ethyl-4-fluorophenyl)-3-(2-methyl-6- oxo-1,6-dihydropyridin-3-yl)-6- (trifluoromethyl)-2,3-dihydropyrido[3,4- d]pyrimidin-4(1H)-one | 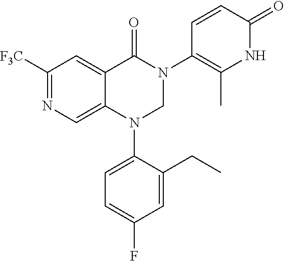 [SMILES: CCc1cc(F)ccc1N1CN(c2ccc(=O)[nH]c2C)C(=O)c2cc(C(F)(F)F)ncc21] |
| 6-Chloro-1-(4-fluoro-2-methylphenyl)-3-(2- methyl-6-oxo-1,6-dihydropyridin-3-yl)-2,3- dihydropyrido[3,4-d]pyrimidin-4(1H)-one | 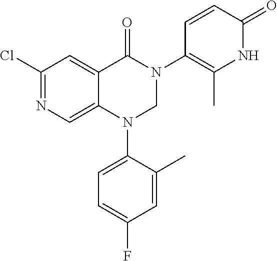 [SMILES: Cc1cc(F)ccc1N1CN(c2ccc(=O)[nH]c2C)C(=O)c2cc(Cl)ncc21] |
| 1-(2-Methyl-4-(trifluoromethoxy)phenyl)-3- (2-methyl-6-oxo-1,6-dihydropyridin-3-yl)-6- (trifluoromethyl)-2,3-dihydropyrido[3,4- d]pyrimidin-4(1H)-one | 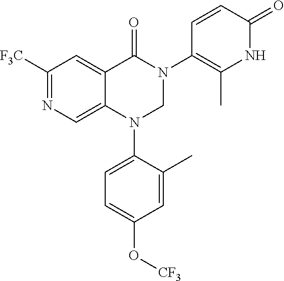 [SMILES: Cc1cc(OC(F)(F)F)ccc1N1CN(c2ccc(=O)[nH]c2C)C(=O)c2cc(C(F)(F)F)ncc21] |
| 1-(3,4-Difluoro-2-methylphenyl)-3-(2- methyl-6-oxo-1,6-dihydropyridin-3-yl)-6- (trifluoromethyl)-2,3-dihydropyrido[3,4- d]pyrimidin-4(1H)-one | 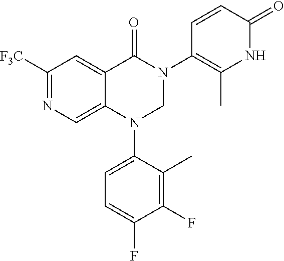 [SMILES: Cc1[nH]c(=O)ccc1N1CN(c2ccc(F)c(F)c2C)c2cnc(C(F)(F)F)cc2C1=O] |
| 7-Chloro-1-(4-fluoro-2-methylphenyl)-3-(2- methyl-6-oxo-1,6-dihydropyridin-3-yl)-2,3- dihydropyrido[4,3-d]pyrimidin-4(1H)-one | 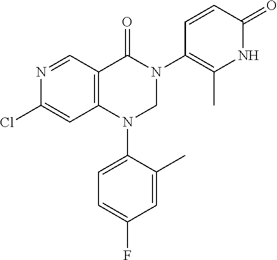 [SMILES: Cc1cc(F)ccc1N1CN(c2ccc(=O)[nH]c2C)C(=O)c2cnc(Cl)cc21] |
| 1-(4-Fluoro-2-methylphenyl)-3-(2-methyl-6- oxo-1,6-dihydropyridin-3-yl)-4-oxo-1,2,3,4- tetrahydropyrido[4,3-d]pyrimidine-7- carbonitrile | 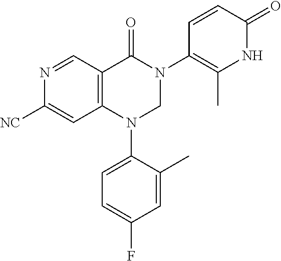 [SMILES: Cc1cc(F)ccc1N1CN(c2ccc(=O)[nH]c2C)C(=O)c2cnc(C#N)cc21] |
| 1-(2-Ethyl-4-fluorophenyl)-3-(2-methyl-6- oxo-1,6-dihydropyridin-3-yl)-7- (trifluoromethyl)-2,3-dihydropyrido[4,3- d]pyrimidin-4(1H)-one | 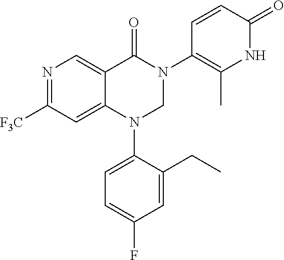 [SMILES: CCc1cc(F)ccc1N1CN(c2ccc(=O)[nH]c2C)C(=O)c2cnc(C(F)(F)F)cc21] |
| 3-(5-Fluoro-2-methyl-6-oxo-1,6- dihydropyridin-3-yl)-1-(2-methyl-4- (trifluoromethoxy)phenyl)-6- (trifluoromethyl)-2,3-dihydropyrido[3,4- d]pyrimidin-4(1H)-one | 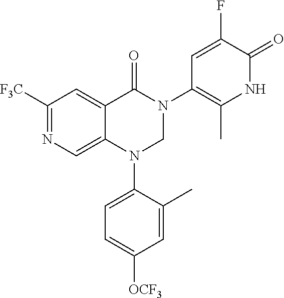 [SMILES: Cc1cc(OC(F)(F)F)ccc1N1CN(c2cc(F)c(=O)[nH]c2C)C(=O)c2cc(C(F)(F)F)ncc21] |
| 1-(4-Fluoro-2-methylphenyl)-3-(2-methyl-6- oxo-1,6-dihydropyridin-3-yl)-4-oxo-1,2,3,4- tetrahydropyrido[3,4-d]pyrimidine-6- carbonitrile | 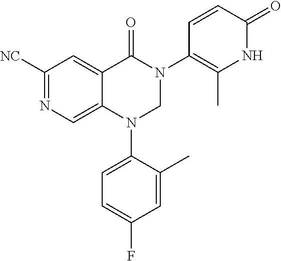 [SMILES: Cc1cc(F)ccc1N1CN(c2ccc(=O)[nH]c2C)C(=O)c2cc(C#N)ncc21] |
| 3-Methyl-4-(1-(2-methyl-4- (trifluoromethoxy)phenyl)-4-oxo-6- (trifluoromethyl)-1,4-dihydropyrido[3,4- d]pyrimidin-3(2H)-yl) pyridine 1-oxide | 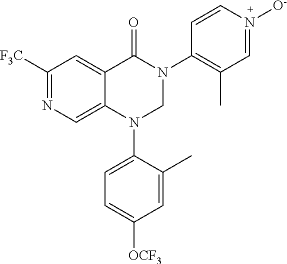 |

or a tautomer thereof, or a pharmaceutically acceptable salt thereof.

In another aspect the invention relates to a compound which is:

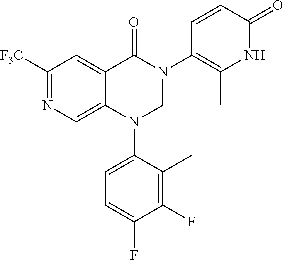 [SMILES: Cc1[nH]c(=O)ccc1N1CN(c2ccc(F)c(F)c2C)c2cnc(C(F)(F)F)cc2C1=O]

or a tautomer thereof, or a pharmaceutically acceptable salt thereof.

Enantiomers, Diastereomers, and Polymorphs

The compounds according to any of the Formulas disclosed herein, including Formulas (I) and (I-A) to (I-E), or a pharmaceutically acceptable salt and/or a corresponding tautomer form thereof of the invention, may contain one or more asymmetric center(s) (i.e., also referred to as a chiral center) and may, therefore, exist in optically forms (e.g., as individual enantiomers, diastereomers, or other stereoisomeric forms, or as mixtures thereof) and racemic forms. All of these individual compounds, stereoisomers, and mixtures thereof are included within the scope of the invention.

Chiral centers, such as chiral carbon atoms, may also be present in a substituent such as an alkyl group. Where the stereochemistry of a chiral center present in any of the Formulas disclosed herein, including Formulas (I) and (I-A) to (I-E), or a pharmaceutically acceptable salt and/or a corresponding tautomer form thereof of the invention, or in any chemical structure illustrated herein, is not specified the structure is intended to encompass all individual stereoisomers and all mixtures thereof. Thus, compounds or a pharmaceutically acceptable salt and/or a corresponding tautomer form thereof of the invention containing one or more chiral centers may be used as racemic mixtures, enantiomerically enriched mixtures, or as enantiomerically pure individual stereoisomers.

Individual stereoisomers of a compound according to any of the Formulas disclosed herein, including Formulas (I) and (I-A) to (I-E), or a pharmaceutically acceptable salt and/or a corresponding tautomer form thereof of the invention, which contain one or more asymmetric centers may be resolved by methods known to those skilled in the art. For example, such resolution may be carried out:
(1) by formation of diastereoisomeric salts, complexes or other derivatives;
(2) by selective reaction with a stereoisomer-specific reagent, for example by enzymatic oxidation or reduction; or
(3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form.

Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.”

It is to be understood that when named or depicted by structure, the disclosed compound, or solvates (particularly, hydrates) thereof, also include all polymorphs thereof.

Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

In one aspect, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one or a tautomer thereof, or a pharmaceutically acceptable salt thereof in crystalline form.

In one embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one in crystalline form.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD (X-ray powder diffraction) pattern having peaks (°2θ) at about 6.3, about 7.4, about 10.0 and/or about 12.6.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Form 1 comprising peaks substantially as set out in Table 1.

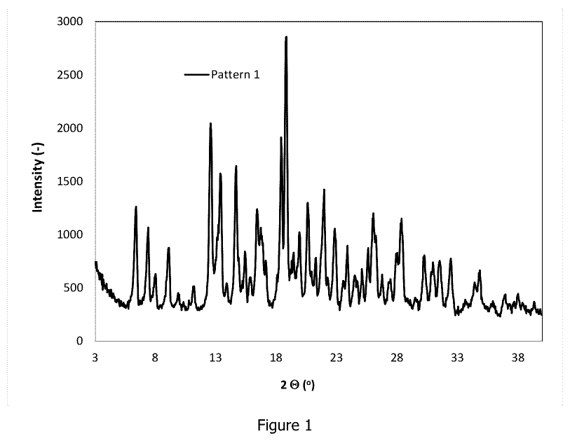

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Pattern 1 substantially in accordance with FIG. 1.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides a DSC endotherm onset at about 49° C.

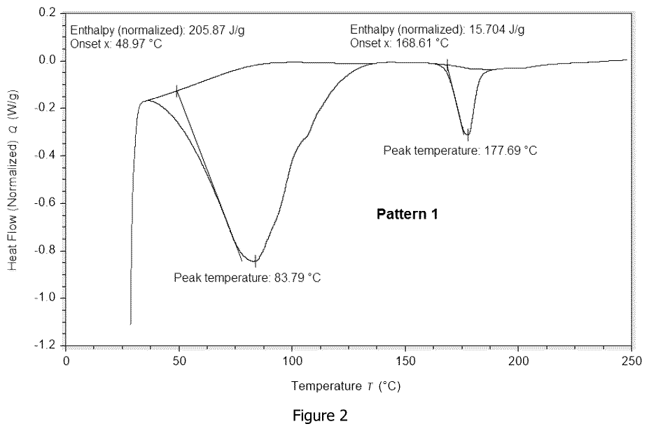

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides a DSC substantially in accordance with FIG. 2.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD (X-ray powder diffraction) pattern having peaks (°2θ) at about 6.9, about 8.2, about 10.8, about 13.5 and/or about 14.8.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Form 2 comprising peaks substantially as set out in Table 1.

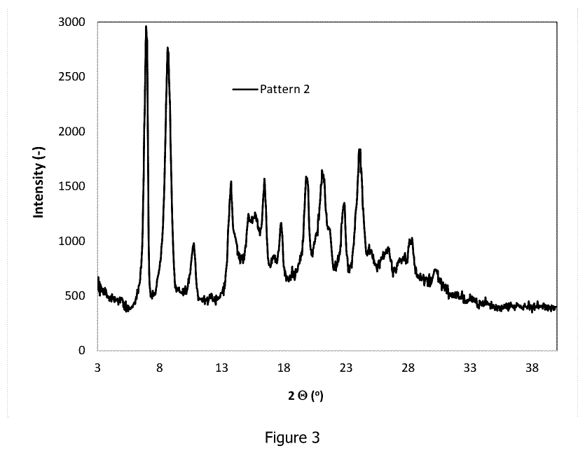

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Pattern 2 substantially in accordance with FIG. 3.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides a DSC endotherm onset at about 41° C.

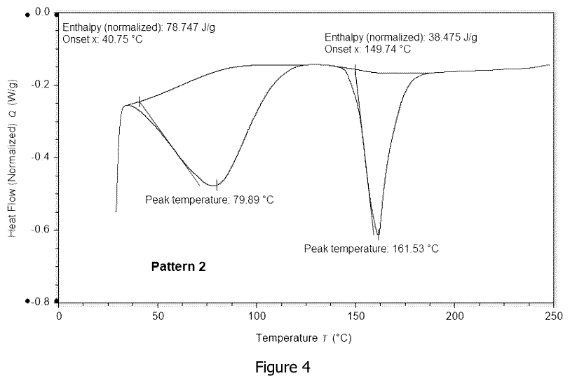

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides a DSC substantially in accordance with FIG. 4.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD (X-ray powder diffraction) pattern having peaks (°2θ) at about 7.1, about 9.3, about 10.2, about 13.8 and/or about 15.0.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Form 3 comprising peaks substantially as set out in Table 1.

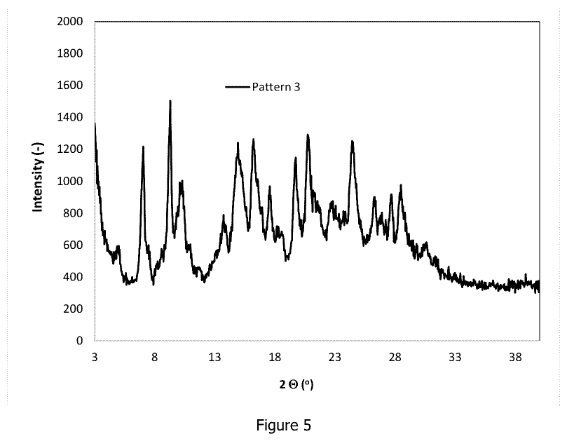

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Pattern 3 substantially in accordance with FIG. 5.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD (X-ray powder diffraction) pattern having peaks (°2θ) at about 3.9, about 7.0, about 7.3, about 7.6 and/or about 9.0.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Form 4 comprising peaks substantially as set out in Table 1.

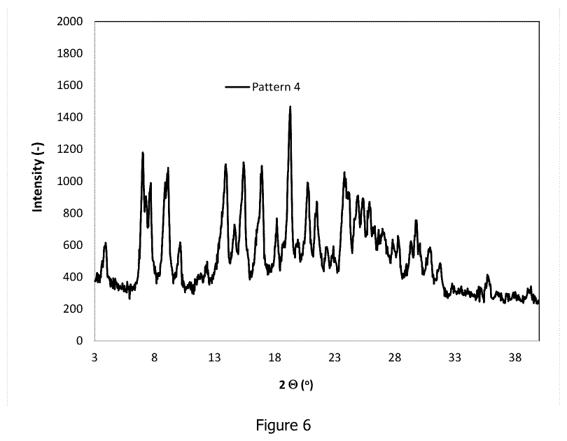

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides an XRPD Pattern 4 substantially in accordance with FIG. 6.

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides a DSC endotherm onset at about 42° C.

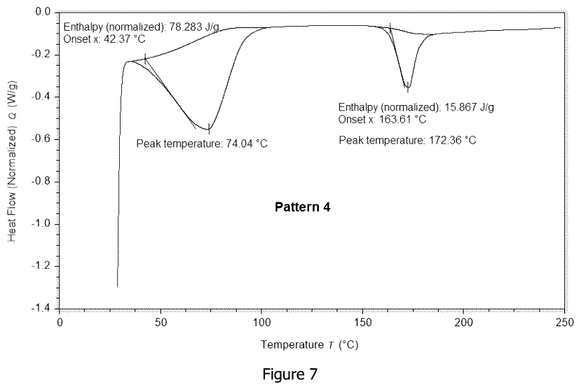

In another embodiment, the present invention provides 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one characterised in that it provides a DSC substantially in accordance with FIG. 7.

When it is indicated herein that there is a peak in an XRPD pattern at a given value, it is typically meant that the peak is within ±0.2, for example ±0.1, of the value quoted.

When it is indicated herein that there is a temperature in a DSC at a given value, it is typically meant that the temperature is within 0.2° C., for example 0.1° C., of the value quoted.

Salts

Because of their potential use in medicine, the salts of the compounds of any of the Formulas disclosed herein, including Formulas (I) and (I-A) to (I-E) and/or corresponding tautomer forms thereof of the invention, are preferably pharmaceutically acceptable salts. Pharmaceutically acceptable salts include, among others, those described by Berge, Bighley and Monkhouse J. Pharm. Sci (1977) 66, pp 1-19, or those listed in PH Stahl and CG Wermuth, editors, Handbook of Pharmaceutical Salts; Properties, Selection and Use, Second Edition Stahl/Wermuth: Wiley-VCH/VHCA, 2011. Non-pharmaceutically acceptable salts may be used, for example as intermediates in the preparation of a compound of any of the Formulas disclosed herein or a pharmaceutically acceptable salt thereof.

Suitable pharmaceutically acceptable salts can include acid or base addition salts. Such base additional salts can be formed by reaction of a compound of any of the Formulas disclosed herein, including Formulas (I) and (I-A) to (I-E) and/or corresponding tautomer forms thereof of the invention with the appropriate base, optionally in a suitable solvent such as an organic solvent, to give the salt which can be isolated by a variety of methods, including crystallisation and filtration.

Such acid addition salts can be formed by reaction of a compound of any of the Formulas disclosed herein, including Formulas (I) and (I-A) to (I-E) and/or corresponding tautomer forms thereof of the invention, with the appropriate acid, optionally in a suitable solvent such as an organic solvent, to give the salt which can be isolated by a variety of methods, including crystallisation and filtration.

Salts may be prepared in situ during the final isolation and purification of a compound of any of the Formulas disclosed herein, including Formulas (I) and (I-A) to (I-E) and/or corresponding tautomer forms thereof of the invention. If a basic compound of any of the Formulas disclosed herein, including Formulas (I) and (I-A) to (I-E) and/or corresponding tautomer forms thereof of the invention, is isolated as a salt, the corresponding free base form of that compound may be prepared by any suitable method known to the art, including treatment of the salt with an inorganic or organic base. Similarly, if a compound of any of the Formulas disclosed herein, including Formulas (I) and (I-A) to (I-E) and/or corresponding tautomer forms thereof of the invention, containing a carboxylic acid or other acidic functional group is isolated as a salt, the corresponding free acid form of that compound may be prepared by any suitable method known to the art, including treatment of the salt with an inorganic or organic acid.

For example, when a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like.

If an inventive basic compound is isolated as a salt, the corresponding free base form of that compound may be prepared by any suitable method known to the art, including treatment of the salt with an inorganic or organic base, suitably an inorganic or organic base having a higher pKa than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms. It will be understood that if a compound of any of the Formulas disclosed herein, including Formulas (I) and (I-A) to (I-E) defined herein contains two or more basic moieties, the stoichiometry of salt formation may include 1, 2 or more equivalents of acid. Such salts would contain 1, 2 or more acid counterions, for example, a dihydrochloride salt. Stoichiometric and non-stoichiometric forms of a pharmaceutically acceptable salt of a compound of any of the Formulas disclosed herein, including Formulas (I) and (I-A) to (I-E) and/or corresponding tautomer forms thereof of the invention are included within the scope of the invention, including sub-stoichiometric salts, for example where a counterion contains more than one acidic proton.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

Solvates

Compounds of the invention, or pharmaceutically acceptable salts thereof may exist in solvated and unsolvated forms. For solvates of the compounds of the invention, or pharmaceutically acceptable salts thereof or tautomers thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water.

Deuterated Compounds

The invention also includes various deuterated forms of the compounds of any of the Formulas disclosed herein, including Formulas (I) and (I-A) to (I-E) or a pharmaceutically acceptable salt and/or a corresponding tautomer form thereof of the invention. Each available hydrogen atom attached to a carbon atom may be independently replaced with a deuterium atom.

A person of ordinary skill in the art will know how to synthesize deuterated forms of the compounds of any of the Formulas disclosed herein, including Formulas (I) and (I-A) to (I-E) or a pharmaceutically acceptable salt and/or a corresponding tautomer form thereof of the invention. For example, deuterated materials, such as alkyl groups may be prepared by conventional techniques (see for example: methyl-d₃-amine available from Aldrich Chemical Co., Milwaukee, WI, Cat. No. 489, 689-2).

Isotopes

The invention also includes isotopically-labeled compounds which are identical to those recited in any of the Formulas disclosed herein, including Formulas (I) and (I-A) to (I-E) or a pharmaceutically acceptable salt and/or a corresponding tautomer form thereof of the invention but for the fact that one or more atoms are replaced by an atom having an atomic mass or mass number different from the atomic mass or mass number most commonly found in nature.

Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the invention and pharmaceutically acceptable salts of said compounds that contain the aforementioned isotopes and/or other isotopes of other atoms are within the scope of the invention. Isotopically labeled compounds of the invention, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Purity

Because the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing more pure forms used in the pharmaceutical compositions.

It is recognized that the compounds of any of the Formulas disclosed herein, including Formulas (I) and (I-A) to (I-E) or a pharmaceutically acceptable salt and/or a corresponding tautomer form thereof of the invention may exist in forms as stereoisomers, regioisomers, or diastereoisomers.

Tautomers

Moreover, compounds of the invention may exist as tautomers or in tautomeric forms. It is conventionally understood in the chemical arts that tautomers are structural or constitutional isomers of chemical compounds that readily interconvert. This reaction commonly results in the relocation of a proton. A structural isomer, or constitutional isomer (per IUPAC), is a type of isomer in which molecules with the same molecular formula have different bonding patterns and atomic organization, as opposed to stereoisomers, in which molecular bonds are always in the same order and only spatial arrangement differs. The concept of tautomerizations is called tautomerism. The chemical reaction interconverting the two is called tautomerization. Care should be taken not to confuse tautomers with depictions of ‘contributing structures’ in chemical resonance. Tautomers are distinct chemical species and can be identified as such by their differing spectroscopic data, whereas resonance structures are merely convenient depictions and do not physically exist.

For example, the 2-pyridone ring exhibits tautomerism, wherein the proton attached to the nitrogen can move to the oxygen to give the tautomeric form 2-hydroxypyridine:

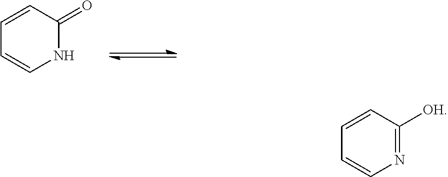 [SMILES: O=c1cccc[nH]1 ; Oc1ccccn1]

Synthetic Schemes and General Methods of Preparation

The present invention also relates to processes for making compounds of any of the Formulas disclosed herein, including Formulas (I) and (I-A) to (I-E) or a pharmaceutically acceptable salt and/or a corresponding tautomer form thereof of the invention.

The compounds of any of the Formulas disclosed herein, or a pharmaceutically acceptable salt and/or a corresponding tautomer form thereof of the invention may be made by any number of processes using conventional organic syntheses as described in the Schemes below and more specifically illustrated by the exemplary compounds which follow in the Examples section herein, or by drawing on the knowledge of a skilled organic chemist. Suitable synthetic routes are depicted below in the following general reaction schemes.

The synthesis provided in these Schemes are applicable for producing compounds of the invention as defined by any of the Formulas disclosed herein, having a variety of different functional groups as defined employing appropriate precursors, which are suitably protected if needed, to achieve compatibility with the reactions outlined herein. Subsequent deprotection, where needed, affords compounds of the nature generally disclosed. While the Schemes are shown with compounds only as defined therein, they are illustrative of processes that may be used to make the compounds of the invention.

Intermediates (compounds used in the preparation of the compounds of the invention) also may be present as salts. Thus, in reference to intermediates, the phrase “compound(s) of formula (number)” means a compound having that structural formula or a pharmaceutically acceptable salt thereof.

The compounds of the invention may be obtained by using the procedures illustrated in the Schemes below, or by applying appropriate synthetic organic chemistry procedures and methodology known to those of skill in the art.

The methods provided in these Schemes can be used to prepare compounds of the invention containing a variety of different Y, X¹, X², X³, X⁴, R¹, R², R³, R⁴, R⁵, R⁶, R⁷, R^{a}, R^{b}, n, R^{7a}, and R^{7b} groups (descriptions shown above for compounds of Formulas (I) and (I-A) to (I-E)) employing appropriate precursors.

Those skilled in the art will appreciate that in the preparation of compounds of the invention (e.g., compounds of Formulas (I) and (I-A) to (I-E) or a pharmaceutically acceptable salt and/or a corresponding tautomer form thereof), it may be necessary and/or desirable to protect one or more sensitive groups in the molecule or the appropriate intermediate to prevent undesirable side reactions. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups for use according to the present invention are well-known to those skilled in the art and may be used in a conventional manner. See for example, “Protective Groups in Organic Synthesis” by T. W. Green and P. G. M Wets (Wiley & Sons, 1991) or “Protecting Groups” by P. J. Kocienski (Georg Thieme Verlag, 1994). Subsequent deprotection, where needed, affords compounds of the nature generally disclosed.

In some instances, a substituent may be specifically selected to be reactive under the reaction conditions used. Under these circumstances, the reaction conditions convert the selected substituent into another substituent that is either useful as an intermediate compound or is a desired substituent in a target compound.

While the Schemes shown below are representative of methods for preparing compounds of Formulas (I) and (I-A) to (I-E), they are only intended to be illustrative of processes that may be used to make the compounds of the invention.

Compound names were generated using the software naming program ChemDraw Ultra v12.0, available from Perkin Elmer, 940 Winter Street, Waltham, Massachusetts, 02451, USA. (http://www.perkinelmer.com/).

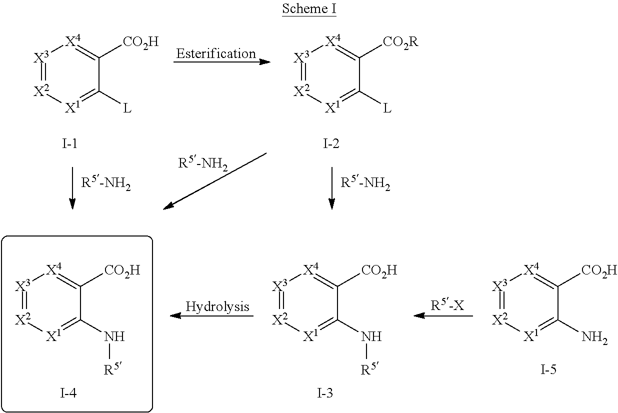

The preparation of the compounds of the invention typically begins with the synthesis of N-substituted-2-aminoaromatic acid derivatives I-4 (Scheme I). Esterification of a suitably substituted 2-halo aromatic acid I-1 under standard conditions provides the corresponding ester I-2. Typically, esterification reactions are performed under either acidic conditions, in the presence of an alcohol, or under basic conditions, in the presence of a suitable alkyl halide. Reaction of the 2-halo aromatic ester I-2 (L=Cl, Br or I) with an appropriate aniline or amine (R^{5′}—NH₂, wherein R^{5′} is a substituted phenyl group) provides the corresponding N-substituted-2-aminoaromatic esters I-3. Typically, this reaction is performed at elevated temperature, using either standard heating or microwave irradiation, in the presence of a catalyst, for example Pd₂(dba)₃ or Cu/CuO, a suitable ligand, for instance BINAP or Xantphos, and an inorganic base, typically Cs₂CO₃ or K₂CO₃, in an appropriate solvent, such as 1,4-dioxane, toluene or 2-ethoxyethanol.

The intermediate I-3 can alternatively be prepared by reacting the 2-aminoaromatic ester I-5 with an appropriate aryl halide (R^{5′}—X, wherein R^{5′} is a substituted phenyl group) under the similar coupling conditions as described above. Such reactions are well-known to those of skill in the art. Saponification of the ester I-3 to the corresponding N-substituted-2-aminoaromatic acid derivatives (I-4) is typically achieved under standard basic conditions, using bases such as LiOH, KOH, or NaOH, in a suitable solvent or solvent system, for instance methanol/H₂O, ethanol/H₂O, or THF/H₂O. Such conditions are well-known to those of skill in the art.

An alternative approach, which will be readily apparent to those of skill in the art, is to react the 2-halo aromatic ester I-1 with an appropriate aniline or amine (R^{5′}—NH₂) to provide compound I-4 directly. The reaction conditions are similar to those described above for conversion of I-2 to I-3. This reaction can also be performed under acidic conditions, such as p-toluenesulfonic acid or acetic acid, at elevated temperature.

The preparation of intermediate I-4 can also be achieved by a coupling reaction of ester I-2 (L=Cl) with an appropriate aniline or amine (R^{5′}—NH₂) under similar coupling conditions as described above for conversion of I-2 to I-3.

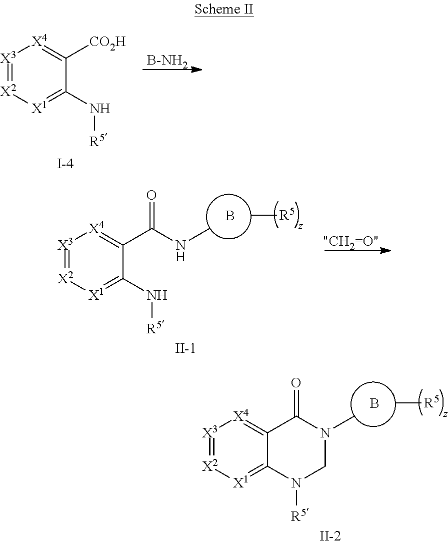

The intermediate N-substituted-2-aminoaromatic acid derivatives I-4, prepared as illustrated in Scheme I, can be converted to II-2 as outlined in Scheme II. Coupling of I-4 with a suitable 2-alkoxy-azaheterocycle B—NH₂, for example 2-methoxy-4-aminopyridine, under various amide coupling conditions known to those of skill in the art, provides the corresponding amide II-1. For example, one might employ standard coupling reagents, like EDC/HOBT, HATU, HBTU or T3P, in the presence of an amine base, like triethylamine, or Hunig's base (diisopropylethylamine), in a suitable solvent, typically DMF, DMA or acetonitrile. Alternatively, one might convert the acid to the corresponding acid chloride, using a reagent like thionyl chloride or oxalyl chloride, then react the acid chloride with a suitable 2-alkoxy-azaheterocycle B—NH₂ (like 2-methoxy-4-aminopyridine), in the presence of an acid scavenger or base, such as pyridine, 2,6-lutidine, triethylamine or Hunig's base, in an appropriate solvent, such as dichloromethane or pyridine, to afford the desired coupling product II-1.

Formation of the ring system, as in II-2, involves reaction of I-1 with formaldehyde or a suitable equivalent. For instance, the reaction may be achieved using formaldehyde, either as gaseous formaldehyde, paraformaldehyde, or s-trioxane, in the presence of an acid, preferably PTSA or sulfuric acid. Alternatively, the ring system can be formed via reaction of II-1 using diiodomethane or chloroiodomethane as a formaldehyde equivalent. In this variant of the cyclization reaction, a base, typically Cs₂CO₃ or NaH, can be used, in a suitable solvent, oftentimes acetonitrile or DMF. The choice of using formaldehyde or diiodomethane depends on the particular reactivity characteristics of the substrate I-1.

In some examples, compound II-2 can be obtained as the final product, which may also be accessed through the method described in Scheme II.

In the instance where ring B in II-2 is substituted by appropriate halogens, particularly chlorine, bromine, or iodine, the halogen can be replaced with other functionalities by reaction with a corresponding coupling partner under appropriate coupling reaction conditions. The coupling partners include suitable amine, alcohol and boronic acid or ester. This type of reaction usually can be realized at elevated temperature, using either standard heating or microwave irradiation, in the presence of a catalyst, usually Pd₂(dba)₃, a suitable ligand, for instance tBuXphos, XPhos or Xantphos, and an inorganic base, typically KOH, Cs₂CO₃ or K₂CO₃, in an appropriate solvent, such as 1,4-dioxane, THF, toluene or 2-ethoxyethanol. In some cases where ring B is substituted with fluorine, the conversion may be achieved through a S_{N}Ar reaction in the presence of a base, for example DIPEA in an appropriate solvent like DMF.

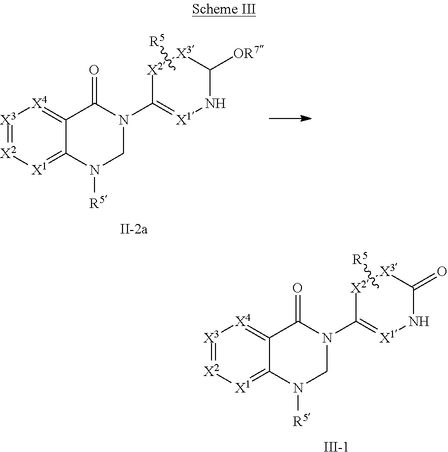

In the instance where B=2-alkoxy-azaheterocycle (each of X¹, X^{2′}, and X³ is independently C or N or NH; R^{7″} is alkyl), for example 6-methoxypyridin-3-amine, removal of the alkoxy (typically methoxy) protecting group may be required to complete the synthesis of the compounds of the invention. Preferred methods for achieving this transformation include reaction with a mixture of TMS-chloride and NaI, or a solution of TMS-iodide, in a neutral solvent like acetonitrile, at elevated temperature. Alternatively, this conversion may be achieved utilizing a mixture of p-toluenesulfonic acid and LiCl in a solvent such as DMF at elevated temperature.

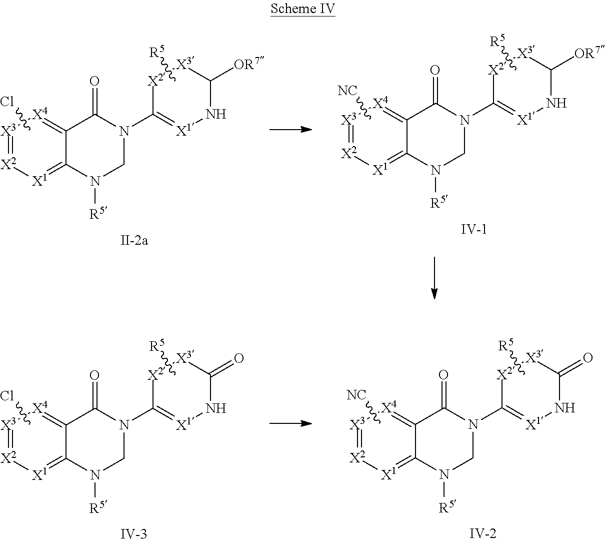

In the instance where one of X¹—X⁴ in II-2a is C—Cl the conversion from the chloro to cyano group can be achieved by treating II-2a with zinc cyanide or copper(I) cyanide in the presence of tetrakis in a solvent such as DMF at elevated temperature to generate the final compound IV-2.

As necessary, the final compound IV-2 can be generated from IV-1 via appropriate deprotection reaction or suitably methods illustrated in Scheme III. The selection of reactions and the corresponding conditions are apparent to those of skill in the art.

Alternatively, the conversion from the chloro group to cyano group can be achieved after the deprotection step utilizing similar reaction conditions as described above for conversion of II-2a to IV-4, to generate the final compound IV-2.

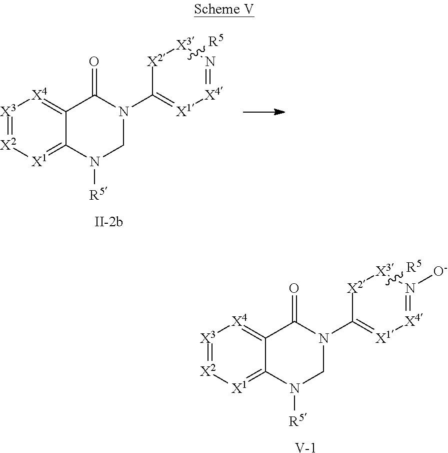

In the instance where B=azaheterocycle such as 3-aminopyridine or 4-aminopyridine etc., an oxidation step may be required to generate the corresponding pyridine N-oxide analogs of the present invention. The conversions are usually achieved in the presence of an oxidant, for example mCPBA, in a neutral solvent (e.g. DCM) at 0° C. or room temperature.

A further deprotection step may be required for some specific examples. Such transformations are well-known to those of skill in the art. For example, in the cases of B ring is 2-alkoxy-azaheterocycle, the alkoxy protecting group can be removed by procedures as described in Scheme IV.

Pharmaceutical Compositions, Administration Routes, and Dosages

The compounds of the invention may be formulated into pharmaceutical compositions prior to administration to a subject. According to one aspect, the invention provides a pharmaceutical composition comprising a compound of the invention (i.e. a compound as defined by any of the Formulas disclosed herein, including Formulas (I) and (I-A) to (I-E) or a pharmaceutically acceptable salt and/or a corresponding tautomer form thereof of the invention) and one or more pharmaceutically acceptable excipients. According to one aspect, the invention provides a pharmaceutical composition comprising a compound of the invention (i.e. a compound as defined by any of the Formulas disclosed herein, including Formulas (I) and (I-A) to (I-E) or a pharmaceutically acceptable salt and/or a corresponding tautomer form thereof of the invention) and a pharmaceutically acceptable excipient.

In another aspect, the invention relates to a pharmaceutical composition or formulation, which comprises: a compound as defined by any of the Formulas disclosed herein, including Formulas (I) and (I-A) to (I-E) or a pharmaceutically acceptable salt and/or a corresponding tautomer form thereof of the invention; a pharmaceutically acceptable excipient(s); and optionally one or more other therapeutic ingredients.

The pharmaceutical compositions or formulations as defined herein typically contain one compound of the invention. However, in certain embodiments, the pharmaceutical compositions may contain more than one compound of the invention. In addition, the pharmaceutical compositions of the invention may optionally further comprise one or more additional pharmaceutically active compounds.

A pharmaceutically acceptable excipient is non-toxic and should not interfere with the efficacy of the active ingredient. Suitable pharmaceutically acceptable excipients will vary depending upon the particular dosage form chosen, route of administration, etc. Suitable pharmaceutically acceptable excipients include the following types of excipients: diluents, carriers, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. Examples of pharmaceutically acceptable excipients are described, e.g., in Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

Pharmaceutical compositions may be adapted for administration by any appropriate or suitable route, for example by systemic administration (e.g., oral administration, parenteral administration, transdermal administration, rectal administration, inhalation), topical administration, etc. Parenteral administration is typically by injection or infusion and includes intravenous, intramuscular, and subcutaneous injection or infusion. Inhalation refers to administration into the patient's lungs whether inhaled through the mouth or through the nasal passages. Typically, administration is via the oral route or parenteral route.

Pharmaceutical compositions adapted for oral administration may be presented as solid dosage forms such as tablets, capsules, caplets, troches, pills; powders; or liquid dosage forms such as solutions, suspensions, syrups, elixirs, or emulsion, etc. Pharmaceutical compositions adapted for parenteral administration may be presented as solutions, suspensions, and powders for reconstitution.

In general, pharmaceutical compositions of the invention are prepared using conventional materials and techniques, such as mixing, blending and the like. Some of the methods commonly used in the art are described in Remington's Pharmaceutical Sciences (Mack Publishing Company).

Solid oral dosage forms, such as tablets and capsules can be prepared by mixing a compound of the invention with excipients such as diluents and fillers (e.g., starch, lactose, sucrose, calcium carbonate, calcium phosphate and the like), binders (e.g., starch, acacia gum, carboxymethyl cellulose, hydroxypropyl cellulose, crystalline cellulose, and the like), lubricants (e.g., magnesium stearate, talc and the like), and the like. Pharmaceutical compositions adapted for parenteral administration can be an injection solution prepared from powders, granules or tablets by mixing with a carrier, such as distilled water, saline and the like, and base and the like may be used for pH adjustment.

The invention also provides a pharmaceutical composition comprising from 0.5 to 1,000 mg of a compound of the invention (i.e., a compound of any of the Formulas disclosed herein, including Formulas (I) and (I-A) to (I-E) or a pharmaceutically acceptable salt and/or a corresponding tautomer form thereof of the invention) and from 0.5 to 1,000 mg of a pharmaceutically acceptable excipient.

Compounds and pharmaceutical compositions of the invention as defined herein may be administered once or according to a dosing regimen, where a number of doses are administered at varying intervals of time for a given period of time. For example, doses may be administered one, two, three, or four times per day. Doses may be administered until the desired therapeutic effect is achieved or indefinitely to maintain the desired therapeutic effect. Doses of compounds of the invention may in the range of 0.001 mg/kg to 100 mg/kg, such as 0.001 mg/kg to 50 mg/kg. Preferably, the selected dose is administered orally or parenterally.

Methods, Uses, Compounds for Use in Manufacture and/or Treatment of Diseases

In general, the invention also relates to uses of the compounds and/or pharmaceutical compositions of the invention as defined herein for use as a medicament or for use in therapy.

Compounds of the invention as defined herein are inhibitors of voltage-gated sodium ion channels, and particularly the voltage-gated sodium ion channel Nav1.8. The activity of a compound utilized in this invention as an inhibitor of Nav1.8 may be assayed according to methods described generally in the Examples herein, or according to methods available to one of ordinary skill in the art.

Accordingly, in one aspect, the invention relates to uses of compounds and pharmaceutical compositions of the invention as inhibitors of voltage-gated sodium ion channels, particularly Nav1.8.

In one embodiment, the invention relates to a method of inhibiting a voltage-gated sodium ion channel in a subject in need thereof, comprising administering to the subject an effective amount of a compound of the invention or a pharmaceutical composition of the invention as described herein. In one embodiment, the voltage-gated sodium channel is Nav1.8.

In embodiment, the invention relates to a compound of the invention or a pharmaceutical composition of the invention for use in inhibiting a voltage-gated sodium ion channel. In one embodiment, the voltage-gated sodium channel is Nav1.8.

In one embodiment, the invention relates to use of a compound of the invention or a pharmaceutical composition of the invention in the manufacture of a medicament for inhibiting a voltage-gated sodium ion channel. In one embodiment, the voltage-gated sodium channel is Nav1.8.

Without wishing to be bound by any particular theory, the compounds and compositions of the invention are particularly useful for treating a disease, condition, or disorder where activation or hyperactivity of Nav1.8 is implicated in the disease, condition, or disorder. When activation or hyperactivity of Nav1.8 is implicated in a particular disease, condition, or disorder, the disease, condition, or disorder may also be referred to as a “Nav1.8-mediated disease, condition or disorder.” Exemplary Nav1.8-mediated diseases, disorders, and conditions include pain and pain-associated diseases, disorders, and conditions, and cardiovascular diseases, disorders, and conditions such as atrial fibrillation.

Thus, in another aspect, the invention relates to uses of compounds and pharmaceutical compositions of the invention in methods and medicaments for treating pain or a pain-associated disease, disorder, or condition and/or for treating cardiovascular diseases, disorders, and conditions.

As used herein, “patient” or “subject” in need thereof refers to a human or mammal. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc. Suitably the subject being treated is a human.

As used herein, the terms “treat”, “treating”, and/or “treatment” used in reference to a disease, disorder, or condition mean to ameliorate or prevent the condition or one or more biological manifestations of the condition; to interfere with one or more points in the biological cascade that leads to or is responsible for the condition; to alleviate one or more of the symptoms or effects associated with the condition; to slow the progression of the condition or one or more of the biological manifestations of the condition; or to lessen the severity of the condition or one or more symptoms or effects associated with the condition. As mentioned above, “treatment” of a disease, disorder, or condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “effective amount” and “therapeutically effective amount” are used interchangeably. An effective amount in reference to a compound of the invention means an amount of the compound sufficient to treat the patient's condition, but low enough to avoid serious side effects (at a reasonable benefit/risk ratio) within the scope of sound medical judgment. An effective amount of a compound or pharmaceutically acceptable salt thereof and/or corresponding tautomer form thereof of the invention or corresponding pharmaceutical composition thereof will vary according to factors, such as the particular compound chosen (e.g., consider the potency, efficacy, and half-life of the compound); the route of administration chosen; the condition being treated; the severity of the condition being treated; the age, size, weight, and physical condition of the patient or subject being treated; the medical history of the patient or subject being treated; the duration of the treatment; the nature of concurrent therapy; the desired therapeutic effect, etc.

According to embodiments of the invention, a pain-associated disease, disorder or condition is pain caused by any one of a variety of diseases of varying etiologies as described throughout the present disclosure. In some embodiments, pain or a pain-associated disease, disorder, or condition is neuropathic pain, chronic pain, acute pain, nociceptive pain, inflammatory pain, musculoskeletal pain, visceral pain, cancer pain, idiopathic pain, multiple sclerosis, Charcot-Marie-Tooth syndrome, or incontinence.

In some embodiments, pain or a pain-associated disease, disorder, or condition is neuropathic pain or chronic neuropathic pain.

In some embodiments, pain or a pain-associated disease, disorder, or condition is neuropathic pain or chronic neuropathic pain selected from small fiber neuropathy, small fiber-mediated diabetic neuropathy, idiopathic small fiber neuropathy, painful diabetic neuropathy or polyneuropathy.

In some embodiments pain or a pain-associated disease, disorder, or condition is neuropathic pain selected from post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion, complex regional pain syndrome, drug therapy induced neuralgia, cancer chemotherapy induced neuralgia, anti-retroviral therapy induced neuralgia, post spinal cord injury pain, idiopathic small-fiber neuropathy, idiopathic sensory neuropathy or trigeminal autonomic cephalalgia.

In some embodiments, pain or a pain-associated disease, disorder, or condition is neuropathic pain or chronic neuropathic pain selected from diabetic peripheral neuropathy, pain caused by neuropathy, neurologic or neuronal injury, pain associated nerve injury, neuralgias and associated acute or chronic pain, post-herpetic neuralgia, pain associated root avulsions, painful traumatic mononeuropathy, painful polyneuropathy, erythromelalgia, paroxysmal extreme pain disorder (PEPD), burning mouth syndrome, central pain syndromes caused by a lesion at a level of nervous system, traumatic nerve injury, nerve compression or entrapment, congenital insensitivity to pain (CIP), dysmenorrheal, primary erythromelalgia, HIV peripheral sensory neuropathy, pudendal neuralgia, spinal nerve injury, chronic inflammatory demyelinating polyneuropathy (CIDP), carpal tunnel syndrome and vasculitic neuropathy.

In some embodiments, pain or a pain-associated disease, disorder, or condition is visceral pain, wherein visceral pain is inflammatory bowel disease pain, Crohn's disease pain or interstitial cystitis pain.

In some embodiments, pain or a pain-associated disease, disorder, or condition is musculoskeletal pain, wherein musculoskeletal pain is osteoarthritis pain, back pain, cold pain, burn pain or dental pain.

In some embodiments, pain or a pain-associated disease, disorder, or condition is idiopathic pain, wherein idiopathic pain is fibromyalgia pain.

In some embodiments, pain or a pain-associated disease, disorder, or condition is chronic or acute pre-operative associated pain or chronic or acute post-operative associated pain. Post-operative associated pain includes ambulatory post-operative pain. Ambulatory surgery, also known as outpatient surgery, refers to same day surgery that does not require an overnight stay in a hospital or other medical facility. In some embodiments, pre-operative associated pain is selected from neuropathic pain or chronic neuropathic pain, chronic osteoarthritis pain, dental pain or inflammatory pain. In some embodiments, post-operative associated pain is selected from bunionectomy pain, hernia repair pair, breast surgery pain or cosmetic surgical pain.

In some embodiments, pain or a pain-associated disease, disorder, or condition is pain caused by trauma or iatrogenic medical or dental procedures. As used herein, the term “iatrogenic” refers to pain induced inadvertently by a medical or dental personnel, such as surgeon or dentist, during medical or dental treatment(s) or diagnostic procedure(s), which include, but are not limited to pain caused by pre-operative (i.e., “before”), peri-operative (i.e., “during” or medically induced pain during non-surgical or operative treatment(s)) and post-operative (i.e., after, post-operative or surgical induced caused pain) medical or dental procedures.

In some embodiments, pain or a pain-associated disease, disorder, or condition is nociceptive pain, wherein nociceptive pain is post-surgical pain, cancer pain, back and craniofacial pain, osteoarthritis pain, dental pain or diabetic peripheral neuropathy.

In some embodiments, pain or a pain-associated disease, disorder, or condition is inflammatory pain. Inflammatory pain can be pain of varied physiological origins. In some embodiments, inflammatory pain is selected from pain associated with osteoarthritis, rheumatoid arthritis, rheumatic disorder, teno-synovitis and gout, shoulder tendonitis or bursitis, gouty arthritis, and polymyalgia rheumatica, primary hyperalgesia, secondary hyperalgesia, primary allodynia, secondary allodynia, or other pain caused by central sensitization; complex regional pain syndrome, chronic arthritic pain and related neuralgias or acute pain. In some embodiments inflammatory pain is selected from pain associated with rheumatoid arthritis, osteoarthritis, rheumatoid spondylitis, gouty arthritis orjuvenile arthritis. In some embodiments, inflammatory pain is selected from rheumatoid arthritis; rheumatoid spondylitis; gouty arthritis; juvenile arthritis; rheumatic disorder; gout; shoulder tendonitis or bursitis; polymyalgia rheumatica; primary hyperalgesia; secondary hyperalgesia; primary allodynia; secondary allodynia; or other pain caused by central sensitization, complex regional pain syndrome, chronic or acute arthritic pain and related neuralgias.

In some embodiments, inflammatory pain is selected from rheumatoid arthritis pain or vulvodynia.

In some embodiments, the inflammatory pain is selected from osteoarthritis, chronic osteoarthritis pain (e.g., hip or knee) or chronic inflammatory demyelinating polyneuropathy.

In some embodiments pain or a pain-associated disease, disorder, or condition is musculoskeletal pain. In some embodiments, musculoskeletal pain is selected from bone and joint pain, osteoarthritis; lower back and neck pain; pain resulting from physical trauma or amputation. In some embodiments, musculoskeletal pain is selected from bone and joint pain, osteoarthritis (e.g., knee, hip), tendonitis (e.g., shoulder), bursitis (e.g., shoulder) tenosynovitis, lower back and neck pain, sprains, strains, or pain resulting from physical trauma or amputation.

In some embodiments, pain or a pain-associated disease, disorder, or condition is neurologic or neuronal injury associated or related pain disorders caused by diseases selected from neuropathy, pain associated nerve injury, pain associated root avulsions, painful traumatic mononeuropathy, painful polyneuropathy, erythromelalgia, paroxysmal extreme pain disorder (PEPD), burning mouth syndrome; central pain syndromes caused by a lesion at a level of nervous system); traumatic nerve injury, nerve compression or entrapment, congenital insensitivity to pain (CIP), dysmenorrheal, primary erythromelalgia; HIV peripheral sensory neuropathy; pudendal neuralgia, spinal nerve injury, chronic inflammatory demyelinating polyneuropathy (CIDP), carpal tunnel syndrome or vasculitic neuropathy.

In some embodiments, pain or a pain-associated disease, disorder, or condition is pain caused by trauma, or pain caused by iatrogenic, medical, or dental procedures.

In some embodiments, pain or a pain-associated disease, disorder, or condition is myofascial pain; myositis or muscle inflammation; repetitive motion pain; complex regional pain syndrome; sympathetically maintained pain; cancer, toxins and chemotherapy related pain; postsurgical pain syndromes and/or associated phantom limb pain; post-operative medical or dental procedures or treatments pain; pain associated with HIV or pain induced by HIV treatment.

In some embodiments, pain or a pain-associated disease, disorder, or condition is neuropathic pain or other pain-associated disease, disorder, or condition selected from peripheral neuropathic pain, central neuropathic pain, inherited erythromelalgia (IEM), small fiber neuralgia (SFN), paroxysmal extreme pain disorder (PEPD), painful diabetic neuropathy, chronic lower back pain, neuropathic back pain, sciatica, non-specific lower back pain, multiple sclerosis pain, HIV-related neuropathy, post-herpetic neuralgia, trigeminal neuralgia, vulvodynia, pain resulting from physical trauma, post-limb amputation pain, neuroma pain, phantom limb pain, cancer, toxins, or chronic inflammatory conditions.

In some embodiments, pain or a pain-associated disease, disorder, or condition is acute pain, chronic pain, neuropathic pain, inflammatory pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, dipolar disorder, myotonia, arrhythmia, movement disorders, neuroendocrine disorders, ataxia, multiple sclerosis, irritable bowel syndrome, incontinence, visceral pain, osteoarthritis pain, postherpetic neuralgia, diabetic neuropathy, radicular pain, sciatica, back pain, head pain, neck pain, severe pain, intractable pain, nociceptive pain, breakthrough pain, postsurgical pain, cancer pain, stroke, cerebral ischemia, traumatic brain injury, amyotrophic lateral sclerosis, stress induced angina, exercise induced angina, palpitations, hypertension, or abnormal gastro-intestinal motility.

In some embodiments, pain or a pain-associated disease, disorder, or condition is femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache, including, cluster headaches; chronic and acute neuropathic pain, post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phantom pain; intractable pain; acute pain, acute post-operative pain; acute musculoskeletal pain; joint pain; mechanical low back pain; neck pain; tendonitis; injury/exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory, burn and trauma pain; acute intermittent pain, endometriosis; acute herpes zoster pain; sickle cell anemia; acute pancreatitis; breakthrough pain; orofacial pain including sinusitis pain, dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease, including, urinary incontinence; hyperactivity bladder; painful bladder syndrome; interstitial cyctitis (IC); prostatitis; complex regional pain syndrome (CRPS), type I and type II; widespread pain, paroxysmal extreme pain, pruritis, tinnitis, or angina-induced pain.

In another aspect, the invention relates to uses of compounds and pharmaceutical compositions of the invention in methods and medicaments for treating cardiovascular diseases, disorders and conditions, including atrial fibrillation and cardiac arrhythmias.

In some embodiments, the cardiovascular disease is atrial fibrillation that is either idiopathic in nature or caused by a disease as defined herein. Atrial fibrillation can be paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease. In particular embodiments, atrial fibrillation is selected from paroxysmal, sustained, or long-standing atrial fibrillation.

In some embodiments, the cardiovascular disease includes cardiac arrhythmias.

In one aspect, the invention relates to a method of treatment of pain or a pain-associated disease, disorder, or condition as defined herein in a subject in need thereof, comprising administering to the subject a therapeutically effective amount of a compound of the invention or a pharmaceutical composition of the invention as described herein.

In an embodiment, provided is a method of treatment of acute pain or chronic pain in a subject in need thereof, comprising administering to the subject a therapeutically effective amount of a compound of the invention or a pharmaceutical composition of the invention as described herein.

In an embodiment, provided is a method of treatment of pain caused by trauma; pain caused by iatrogenic medical or dental procedures; or pre-operative or post-operative associated pain in a subject in need thereof, comprising administering to the subject a therapeutically effective amount of a compound of the invention or a pharmaceutical composition of the invention as described herein.

In an embodiment, provided is a method of treatment of neuropathic pain, nociceptive pain, inflammatory pain, musculoskeletal pain, visceral pain, or idiopathic pain in a subject in need thereof, comprising administering to the subject a therapeutically effective amount of a compound of the invention or a pharmaceutical composition of the invention as described herein.

In an embodiment, provided is a method of treatment of neuropathic pain or chronic neuropathic pain selected from small fiber neuropathy, small fiber-mediated diabetic neuropathy, idiopathic small fiber neuropathy, painful diabetic neuropathy or polyneuropathy in a subject in need thereof, comprising administering to the subject a therapeutically effective amount of a compound of the invention or a pharmaceutical composition of the invention as described herein.

In an embodiment, provided is a method of treatment of inflammatory pain selected from osteoarthritis, chronic osteoarthritis pain, or chronic inflammatory demyelinating polyneuropathy in a subject in need thereof, comprising administering to the subject a therapeutically effective amount of a compound of the invention or a pharmaceutical composition of the invention as described herein.

In one aspect, the invention relates to a method of treatment of atrial fibrillation as defined herein in a subject in need thereof, comprising administering to the subject a therapeutically effective amount of a compound of the invention or a pharmaceutical composition of the invention as described herein.

In one embodiment, provided is a method of treatment of atrial fibrillation, wherein the atrial fibrillation is paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In another aspect, the invention provides compounds of the invention and pharmaceutical compositions of the invention as described herein for use in treatment of pain or a pain-associated disease, disorder, or condition as defined herein.

In an embodiment, provided is a compound of the invention or pharmaceutical composition of the invention for use in treatment of acute pain or chronic pain.

In an embodiment, provided is a compound of the invention or pharmaceutical composition of the invention for use in treatment of pain caused by trauma; pain caused by iatrogenic medical or dental procedures; or pre-operative or post-operative associated pain.

In an embodiment, provided is a compound of the invention or pharmaceutical composition of the invention for use in treatment of neuropathic pain, nociceptive pain, inflammatory pain, musculoskeletal pain, visceral pain, or idiopathic pain.

In an embodiment, provided is a compound of the invention or pharmaceutical composition of the invention for use in treatment of neuropathic pain or chronic neuropathic pain selected from small fiber neuropathy, small fiber-mediated diabetic neuropathy, idiopathic small fiber neuropathy, painful diabetic neuropathy or polyneuropathy.

In an embodiment, provided is a compound of the invention or pharmaceutical composition of the invention for use in treatment of inflammatory pain selected from osteoarthritis, chronic osteoarthritis pain, or chronic inflammatory demyelinating polyneuropathy.

In another aspect, the invention relates to a compound of the invention or a pharmaceutical composition of the invention for use in treatment of atrial fibrillation.

In one embodiment, provided is a compound of the invention or pharmaceutical composition of the invention for use in treatment of atrial fibrillation, wherein the atrial fibrillation is paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In another aspect, the invention also provides uses of compounds of the invention or pharmaceutical compositions of the invention as described herein in the manufacture of a medicament for treatment of pain and pain associated diseases, disorders, and conditions as described herein.

In an embodiment, provided is use of a compound of the invention or pharmaceutical composition of the invention in the manufacture of a medicament for treatment of acute pain or chronic pain.

In an embodiment, provided is use of a compound of the invention or pharmaceutical composition of the invention in the manufacture of a medicament for treatment of pain caused by trauma; pain caused by iatrogenic medical or dental procedures; or pre-operative or post-operative associated pain.

In an embodiment, provided is use of a compound of the invention or pharmaceutical composition of the invention in the manufacture of a medicament for treatment of neuropathic pain, nociceptive pain, inflammatory pain, musculoskeletal pain, visceral pain, or idiopathic pain.

In an embodiment, provided is use of a compound of the invention or pharmaceutical composition of the invention in the manufacture of a medicament for treatment of neuropathic pain or chronic neuropathic pain selected from small fiber neuropathy, small fiber-mediated diabetic neuropathy, idiopathic small fiber neuropathy, painful diabetic neuropathy or polyneuropathy.

In an embodiment, provided is use of a compound of the invention or pharmaceutical composition of the invention in the manufacture of a medicament for treatment of inflammatory pain selected from osteoarthritis, chronic osteoarthritis pain, or chronic inflammatory demyelinating polyneuropathy.

In another aspect, the invention also provides uses of compounds of the invention or pharmaceutical compositions of the invention as described herein in the manufacture of a medicament for treatment of atrial fibrillation.

In an embodiment, provided is use of a compound of the invention or pharmaceutical composition of the invention in the manufacture of a medicament for treatment of atrial fibrillation, wherein the atrial fibrillation is paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In another aspect, the invention relates to a compound of the invention or a pharmaceutical composition of the invention as described herein for use in therapy.

Combination Therapies and Uses for Therapy

Compounds and pharmaceutical compositions of the invention as described herein can be used in combination with one or more additional therapeutic agents. Such additional therapeutic agents can be administered concurrently with, prior to, or subsequent to treatment with a compound or pharmaceutical composition of the invention as described herein.

In the context of this specification, the term “concurrently” when referring to simultaneous administration of compounds or therapeutic agents means at the same time, as would be the case, for example in embodiments where a compound and additional therapeutic agent(s) are combined in a single preparation, or when a compound and additional therapeutic agent(s) are administered separately but taken within a short duration or period of time.

In light of the foregoing, the invention also relates to a combination therapy, which may be a comprised of a simultaneous or co-administration, or serial administration of a combination of compounds or pharmaceutical compositions of the invention with one or more additional therapeutic agents. Such combination therapy can be used for treatment of pain or any pain-associated disease, disorder, or condition, or a cardiovascular disease, disorder, or condition as defined throughout the present specification.

Therapeutic agents suitable for use in combination with the compounds and pharmaceutical compositions of the invention include, but are not limited to: Acetaminophen, Acetylsalicylic acid, Nav1.7 Inhibitors, Nav1.9 Inhibitors, anti-depressants (i.e. such as, but not limited to duloxetine or amitriptyline), anti-convulsants (i.e. such as, but not limited to pregabalin and gabapentin), opiates (i.e., such as, but not limited to hydrocodone, codeine, morphine, oxycodone, oxymorphone, fentanyl, and the like), etc.; and where administration of the above, respectively, also is determined by one of ordinary skill in the art. In one aspect, suitable Nav1.7 Inhibitors or Nav1.9 Inhibitors for use in the invention, include, but are not limited to those Nav1.7 Inhibitors or Nav1.9 Inhibitors known in the chemical literature.

Each component of a combination used for therapeutic purposes (e.g., compound or pharmaceutical composition of the invention and additional therapeutic agent) may be administered orally, intravenously or parenterally or in combinations thereof. Each component of a therapeutic combination may be, but is not limited to being administered by simultaneous administration, co-administration, or serial administration; and/or by identical or different routes of administration or combinations of administration routes. In certain embodiments, each identical or different route of administration or combinations of administration routes is selected from oral, intravenous or parenteral administration.

### EXAMPLES

The following examples illustrate the invention. These examples are not intended to limit the scope of the present invention, but rather to provide guidance to the skilled artisan to prepare and use the compounds, compositions, and methods of the present invention.

While particular aspects or embodiments of the present invention are described, the skilled artisan will appreciate that various changes and modifications can be made without departing from the spirit and scope of the invention.

### Synthesis Examples

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The invention is intended to encompass such salts.

Final compounds were characterized with LCMS or GCMS (conditions listed below) and NMR. ¹H NMR or ¹⁹FNMR spectra were recorded using a BrukerAvance III 500 MHz spectrometer, Bruker Avance 400 MHz spectrometer and Varian Mercury Plus-300 MHz spectrometer. CDCl₃ is deuterochloroform, DMSO-d₆ is hexadeuterodimethylsulfoxide, and CD₃OD is tetradeuteromethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling

Analytical Methods:

1) LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with 0.1% TFA in water (solvent A) and 0.1% TFA in acetonitrile (solvent B), using the following elution gradient: 1-100% (solvent B) over 1.85 min at a flow rate of 1.3 ml/min.

2) LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with formic acid in Water (solvent A) and formic acid in acetonitrile (solvent B), using the following elution gradient: 1-100% (solvent B) over 1.85 min at a flow rate of 1.3 ml/min.

3) LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with 10 mM ammonium bicarbonate in water adjusted to pH=10 with 25% ammonium hydroxide solution (solvent A) and acetonitrile (solvent B), using the following elution gradient: 1-100% (solvent B) over 1.85 min at a flow rate of 1.3 ml/min.

4) LCMS method: Agilent 1290 Infinity II LC system with Agilent MSD 6125B/6130 using multi mode (ESI and APCI+ve and −ve) equipped with a Sunfire C18 column (30 mm×2.1 mm, i.d. 3.5 μm packing diameter) at 25° C. eluting with 0.1% formic acid in water (solvent A) and 0.1% formic acid in acetonitrile (solvent B), using the following elution gradient: 0-100% (solvent B) over 3.1 min and holding at 100% for 0.8 min at a flow rate of 1.0 ml/min.

5) LCMS method: Agilent 1290 Infinity II LC system with Agilent MSD 6125B/6130 using multi mode (ESI and APCI+ve and −ve) equipped with a Atlantis dC18 column (50 mm×4.6 mm, i.d. 5.0 μm packing diameter) at 25° C. eluting with 0.1% TFA in water (solvent A) and methanol (solvent B), using the following elution gradient: 5-95% (solvent B) over 5.0 min and holding at 95% for 1.5 min at a flow rate of 1.0 ml/min.

6) LCMS method: Agilent 1290 Infinity II LC system with Agilent MSD 6125B/6130 using multi mode (ESI and APCI+ve and −ve) equipped with a Zorbax XDB C18 column (50 mm×4.6 mm, i.d. 3.5 μm packing diameter) at 25° C. eluting with 10 mM ammonium acetate in water (solvent A) and acetonitrile (solvent B), using the following elution gradient: Solvent B: 10-95% (solvent B) over 3.5 min and holding at 95% for 1.0 min at a flow rate of 1.0 ml/min.

7) LCMS method: Agilent 1290 Infinity II LC system with Agilent MSD 61251B/6130 using multi mode (ESI and APCI+ve and −ve) equipped with a Xbridge 08 column (50 mm×4.6 mm, i.d. 3.5 μm packing diameter) at 25° C. eluting with 10 mM ammonium bicarbonate in water (solvent A) and acetonitrile (solvent B), using the following elution gradient: 10-95% (solvent B) over 4.0 min and holding at 95% for 1.0 min at a flow rate of 1.0 ml/min.

8) GCMS Method: Agilent 7890B GO system with Agilent MSD 5977B using El equipped with a HP-5 column (30 m×0.32 mm, 0.25 μm film thickness) at 250° C. eluting with helium at a flow rate of 2 mL/min and 10 min run time under the following chromatographic run conditions: 120° C. for 1 min 40° C./min up to 300° C., hold for 4.5 min.

In the following experimental descriptions, the following abbreviations may be used:

| Abbreviation | Meaning |
|---|---|
| AcOH | acetic acid |
| aq. | aqueous |
| BBr₃ | boron tribromide |
| BCl₃ | boron trichloride |
| BH₃ | borane |
| BINAP | 2,2′-bis(diphenylphosphino)-1,1′- |
| | binaphthalene |
| Bn | benzyl |
| brine | saturated aqueous sodium chloride |
| BuLi or nBuLi | butyllithium |
| CDI | carbonyldiimidazole |
| CH₂Cl₂ | methylene chloride |
| CH₃CN | acetonitrile |
| COCl₂ | oxalyl chloride |
| Cs₂CO₃ | cesium carbonate |
| DCC | dicyclohexylcarbodiimide |
| DCM or CH₂Cl₂ | methylene chloride |
| DEAD | diethyl azodicarboxylate |
| DEAP | diethyl aminopyridine |
| DIAD | diisopropyl azodicarboxylate |
| DIPEA, DIEA, Hunig's base | N,N-diisopropylethylamine |
| DMA | Dimethylacetamide |
| DMAP | 4-dimethylaminopyridine |
| DMF | N,N-dimethylformamide |
| DME | dimethoxyethane |
| DMSO | dimethylsulfoxide |
| DSC | Differential scanning calorimetry |
| EDC | 1-[3-(dimethylamino)propyl]-3- |
| | ethylcarbodiimide hydrochloride |
| Et | ethyl |
| Et₃N | triethylamine |
| Et₂O | diethyl ether |
| EtOAc | ethyl acetate |
| EtOH | ethanol |
| Fmoc or fmoc | fluorenylmethyloxycarbonyl |
| g, G, gm, GM | gram |
| GCMS | gas chromatography-mass spectroscopy |
| h or hr | hour(s) |
| H₂ | hydrogen |
| H₂O₂ | hydrogen peroxide |
| H₂O | water |
| H₂SO₄ | sulfuric acid |
| HATU | (O-(7-azabenzotriazol-1-yl)-N,N,N′,N′- |
| | tetramethyluronium hexafluorophosphate) |
| HBTU | 2-(1H-benzo[d][1,2,3]triazol-1-yl)-1,1,3,3- |
| | tetramethylisouronium |
| | hexafluorophosphate(V) |
| HCl | hydrochloric acid |
| HCO₂H | formic acid |
| HOBt or HOBT | 1-hydroxybenzotriazole |
| HPLC | high performance liquid chromatography |
| I₂ | Iodine |
| JLR | jacketed lab reactor |
| K₂CO₃ | potassium carbonate |
| KHSO₄ | potassium hydrogen sulfate |
| KOAc | potassium acetate |
| L or l | liter |
| LAH | lithium aluminum hydride |
| LCMS | liquid chromatography-mass spectroscopy |
| LDA | lithium diisopropyl amide |
| LED | light-emitting diode |
| LiOH | lithium hydroxide |
| LHMDS | lithium bis(trimethylsilyl)amide |
| mCPBA | meta-chloroperoxybenzoic acid |
| MDAP | mass directed autopurification |
| Me | methyl |
| MeOH | methanol |
| mg, MG | milligram |
| MgBr₂ | magnesium bromide |
| MgSO₄ | magnesium sulfate |
| Min or mins | minute(s) |
| ml or mL or ML | milliliter |
| mmol | millimole |
| MnO₂ | manganese dioxide |
| Mol, mol | mole |
| MS | mass spectrum |
| MTBE | methyl tert-butyl ether |
| μw | microwave |
| N₂ | nitrogen |
| Na(CN)BH₃ | sodium cyanoborohydride |
| NaCl | sodium chloride |
| Na₂CO₃ | sodium carbonate |
| NaHCO₃ | sodium bicarbonate |
| NaHMDS | sodium bis(trimethylsilyl)amide |
| NaHSO₃ | sodium bisulfite |
| NaH | sodium hydride |
| Nal | sodium iodide |
| NaOH | sodium hydroxide |
| Na₂SO₃ | sodium sulfite |
| Na₂SO₄ | sodium sulfate |
| NH₄Cl | ammonium chloride |
| HCO₂•NH₄ | ammonium formate |
| NH₄OH | ammonium hydroxide |
| nm | nanometer |
| NMO | 4-methylmorpholine N-oxide |
| NMP | N-methyl-2-pyrrolidone |
| Pd/C | palladium on carbon |
| PdCl₂(dbpf) | 1,1′-bis(di-tert-butylphosphino)ferrocene |
| | dichloropalladium |
| Pd(dppf)Cl₂/PdCl₂(dppf) | [1,1′-bis(diphenylphosphino)ferrocene] |
| | dichloropalladium(II) |
| PdCl₂(dppf)-CH₂Cl₂ adduct | [1,1′-bis(diphenylphosphino)ferrocene] |
| | dichloropalladium(II), complex with |
| | dichloromethane |
| Pd₂ (dba)₃ | tris(dibenzylideneacetone)dipalladium(0) |
| Pd(Ph₃)₄, tetrakis | tetrakis(triphenylphosphine)palladium(0) |
| PdOAc₂ or Pd(OAc)₂ | palladium acetate |
| Pd(OH)₂ | palladium hydroxide |
| PIFA | [Bis(trifluoroacetoxy)iodo]benzene |
| Ph | phenyl |
| PL HCO₃ MP | macroporus polystyrene supported |
| | carbonate |
| POCl₃ | phosphoryl chloride |
| psi | Pounds per square inch |
| PTFE | polytetrafluoroethylene |
| PTSOH or PTSA or pTsOH | p-Toluenesulfonic acid |
| rt or RT | room temperature |
| sat. | saturated |
| SFC | supercritical fluid chromatography |
| Si | silica |
| Si SPE | silica gel cartridges |
| SiO₂ | silica gel |
| SPE | solid phase extraction |
| T3P ® | propylphosphonic anhydride |
| tBu or t-Bu | tert-butyl group |
| TBAB | tetrabutylammonium bromide |
| TBAF | tetrabutylammonium fluoride |
| TBAI | tetrabutylammonium iodide |
| TBDMSCl | tert-butyldimethylsilyl chloride |
| TBME | tert-butylmethyl ether |
| TBTU | 2-(1H-benzotriazole-1-yl)-1,1,3,3- |
| | tetramethyluronium tetrafluoroborate |
| TEA | triethylamine |
| TFA | trifluoroacetic acid |
| THF | tetrahydrofuran |
| TiCl₄ | titanium tetrachloride |
| TMS-Br or TMSBr | trimethylsilyl bromide |
| TMS-Cl or TMSCl | trimethylsilyl chloride |
| TMSI | Iodotrimethylsilane or trimethylsilyl iodide |
| TMS-OTf or TMSOtf | trimethylsilyl triflate |
| tR | retention time |
| UPLC | ultra performance liquid chromatography |
| Xantphos | 4,5-Bis(diphenylphosphino)-9,9- |
| | dimethylxanthene |
| Xphos | 2-Dicyclohexylphosphino-2′,4′,6′- |
| | triisopropylbiphenyl |
| XRPD | X-ray powder diffraction |

### Intermediate 1

### Methyl 5-bromo-2-chloroisonicotinate

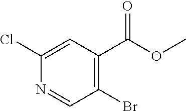

To a stirring solution of 5-bromo-2-chloroisonicotinic acid (3 g, 12.69 mmol) in methanol (25 mL) at 0° C. was added thionyl chloride (2.78 mL, 38.1 mmol) and the reaction mixture was heated at 80° C. for 3 h. The reaction was cooled and concentrated. The resultant brown solid was diluted with water (50 mL) and extracted with EtOAc (2×25 mL). The combined organic extracts were washed with water (10 mL) and brine (10 mL), dried over Na₂SO₄ and concentrated to give the title compound as a brown oil (1.88 g, 7.40 mmol, 58.3% yield). MS (m/z) 251.9 (M+3H)⁺.

### Intermediate 2

### Ethyl 4-bromo-6-(trifluoromethyl)nicotinate

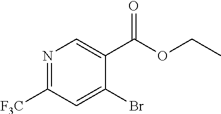 [SMILES: CCOC(=O)c1cnc(C(F)(F)F)cc1Br]

To a stirring solution of 4-bromo-6-(trifluoromethyl)nicotinic acid (1 g, 3.70 mmol) and ethyl iodide (0.329 mL, 4.07 mmol) in DMF (10 mL) under N₂ at RT was added potassium carbonate (0.614 g, 4.44 mmol). After stirring at 27° C. for 3 h, the reaction mixture was diluted with ice cold water (250 mL) and extracted with Et₂O (2×100 mL). The combined organic extracts were washed with ice-cold water (200 mL), dried over Na₂SO₄ and concentrated under reduced pressure to give the title compound as a brown oil (1.1 g, 3.21 mmol, 87% yield). MS (m/z) 300.0 (M+3H)⁺.

Intermediate 3 was prepared from the indicated carboxylic acid by methods analogous to those described for Intermediate 2.

| Int. | Name | Structure | Characterization | Carboxylic acid |
|---|---|---|---|---|
| 3 | ethyl 3-bromo-6- chloropicolinate | 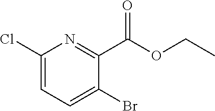 | MS (m/z) 265.9 (M + 3H)⁺ | 3-bromo-6- chloropicolinic acid |

### Intermediate 4

### 3-Bromo-6-methoxypicolinic acid

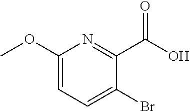

To a stirring solution of ethyl 3-bromo-6-chloropicolinate (1.5 g, 5.67 mmol) in methanol (5 mL) was added sodium methoxide (25% wt in methanol) (7.78 mL, 34.0 mmol) dropwise over 2 minutes at 0° C. After stirring at 60° C. for 6 hr, solvent was removed under vacuum and the pH of the residue was adjusted to 5-6 with aqueous citric acid solution. The reaction was diluted with water (100 mL) and extracted with EtOAc (2×100 mL). The combined organic extracts were concentrated under vacuum to give the title compound as an off-white solid (1.02 g, 3.85 mmol, 67.9% yield). MS (m/z) 234.0 (M+3H)⁺.

### Intermediate 5

### Ethyl 3-bromo-6-methoxypicolinate

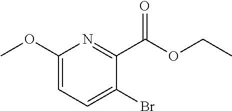

This intermediate was prepared from 3-bromo-6-methoxypicolinic acid by methods analogous to those described for Intermediate 2. MS (m/z) 262.0 (M+3H)⁺.

### Intermediate 6

### Ethyl 5-bromo-2-(trifluoromethyl)isonicotinate

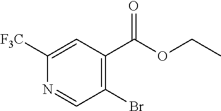 [SMILES: CCOC(=O)c1cc(C(F)(F)F)ncc1Br]

To a suspension of 5-bromo-2-(trifluoromethyl)isonicotinic acid (500 mg, 1.852 mmol) in ethanol (3 mL) was added sulfuric acid (0.296 mL, 5.56 mmol) resulting in an exotherm. The reaction mixture was sealed, stirred and heated to 70° C. After ˜5 minutes a solution had formed and stirring was continued for another 4 h. The reaction mixture was diluted with water (7 mL), basified with 2M NaOH (aq), followed by extraction into EtOAc (2×5 mL). The combined organics were dried by filtration through a hydrophobic frit and concentrated to give the title compound as a pale yellow oil/solid (443 mg, 1.486 mmol, 80% yield). MS (m/z) 299.9 (M+3H)⁺.

### Intermediate 7

### Ethyl 3-amino-3-imino-2-nitrosopropanoate

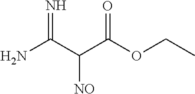

To a stirring solution of ethyl 3-amino-3-iminopropanoate, hydrochloride (10 g, 60.0 mmol) in water (30 mL) at 0° C., acetic acid (10.31 mL, 180 mmol) and sodium nitrite (12.42 g, 180 mmol) were added. After stirring for 16 hr at RT, the solid precipitate was filtered and dried under vacuum to give the title compound as a yellow solid (6 g, 32.4 mmol, 54.0% yield). MS (m/z) 160.2 (M+H)⁺.

### Intermediate 8

### Ethyl 2,3-diamino-3-iminopropanoate, 2Hydrochloride

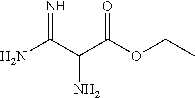

To a stirring solution of ethyl 3-amino-3-imino-2-nitrosopropanoate (6 g, 37.7 mmol) in ethanol (120 mL) and aq.HCl (5M) (120 ml, 600 mmol) under N₂ was added Pd/C (10% wt.) (1.605 g, 1.508 mmol). The reaction mixture was stirred under hydrogen (1 atm) at RT for 40 h, filtered through a pad of Celite® and concentrated. The resultant off-white solid was dissolved in EtOH (60 mL) and aq. HCl (60 mL). Pd/C (10% wt) (0.8 g, 0.752 mmol) was added under N₂ and the reaction was stirred under hydrogen (1 atm) at RT for 16 h. The reaction mixture was filtered through a pad of Celite® and concentrated to give the title compound as an off-white solid (5.5 g, 24.97 mmol, 66.2% yield). MS (m/z) 146.1 (M+H)⁺.

### Intermediate 9 and Intermediate 10

### Ethyl 3-amino-5-(trifluoromethyl)pyrazine-2-carboxylate and Ethyl 3-amino-6-(trifluoromethyl)pyrazine-2-carboxylate

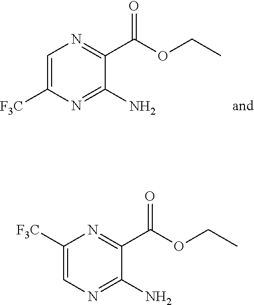 [SMILES: CCOC(=O)c1ncc(C(F)(F)F)nc1N ; CCOC(=O)c1nc(C(F)(F)F)cnc1N]

To a stirring solution of ethyl 2,3-diamino-3-iminopropanoate (4 g, 27.6 mmol) in water (200 mL), 3,3,3-trifluoro-2-oxopropanal (20% wt in water) (24.31 g, 38.6 mmol) and sodium acetate (15.82 g, 193 mmol) were added. The reaction mixture was stirred at RT for 2 hr and then extracted with EtOAc (300 mL). The organic layer was washed with brine (50 mL), dried over Na₂SO₄, filtered and concentrated. The crude product was purified by column chromatography (Biotage Isolera, 100 g Si₂O SNAP column, 3% EtOAc/petroleum ether over 15 minutes) to give

Ethyl 3-amino-5-(trifluoromethyl)pyrazine-2-carboxylate as an off-white solid (750 mg, 3.16 mmol, 11.46% yield). MS (m/z) 236.1 (M+H)⁺

Ethyl 3-amino-6-(trifluoromethyl)pyrazine-2-carboxylate as an off-white solid (440 mg, 1.852 mmol, 6.72% yield). MS (m/z) 236.0 (M+H)⁺

### Intermediate 11

### 4-Fluoro-1-nitro-2-(prop-1-en-2-yl)benzene

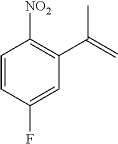

A 2.5 L 4-neck round bottom flask equipped with a magnetic stir bar and N₂ inlet was charged with 2-chloro-4-fluoro-1-nitrobenzene (90 g, 513 mmol), 1,4-Dioxane (1000 mL), sodium carbonate (65.2 g, 615 mmol), water (200 mL), 4,4,5,5-tetramethyl-2-(prop-1-en-2-yl)-1,3,2-dioxaborolane (103 g, 615 mmol) and PdCl₂(dppf)-CH₂Cl₂ adduct (20.93 g, 25.6 mmol). The flask was purged with N₂ for 30 minutes and then stirred at 80° C. for 16 hr. The reaction mixture was cooled to RT, purged with N₂ for 20 minutes, added PdCl₂(dppf)-CH₂Cl₂ adduct (2.093 g, 2.56 mmol) and stirred at 80° C. for another 16 hr. The reaction mixture was cooled to RT and filtered through a Celite® pad washing with EtOAc (300 mL). The filtrate was concentrated and the residue was washed with water (500 mL), dried over Na₂SO₄ and concentrated in vacuo to give the title compound as a brown oil (120 g, 489 mmol, 95% yield). GCMS (m/z) 181 (M)⁺

### Intermediate 12

### 3,4-Difluoro-2-vinylaniline

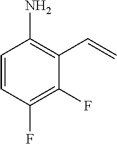

This intermediate was prepared by methods analogous to those described for Intermediate 11 using 2-bromo-3,4-difluoroaniline and 4,4,5,5-tetramethyl-2-vinyl-1,3,2-dioxaborolane in place of 2-chloro-4-fluoro-1-nitrobenzene and 4,4,5,5-tetramethyl-2-(prop-1-en-2-yl)-1,3,2-dioxaborolane. MS (m/z) 156.0 (M+H⁺).

### Intermediate 13

### 4-Fluoro-2-isopropylaniline

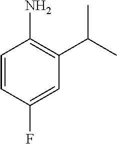

A solution of 4-fluoro-1-nitro-2-(prop-1-en-2-yl)benzene (120 g, 662 mmol) in EtOAc (2 L) was hydrogenated over Pd/C (10% wt) (30 g, 28.2 mmol) under 1 atm hydrogen pressure at RT for 16 hr. The reaction mixture was filtered through a Celite® bed washing with EtOAc (2 L). The filtrate was concentrated and purified by column chromatography (Biotage Isolera, 340 g Si₂O column, 0-20% EtOAc/petroleum ether over 4 h) to give the title compound as a brown oil (70 g, 395 mmol, 59.6% yield). MS (m/z) 154.1 (M+H)⁺.

Intermediates 14-15 were prepared from the indicated aryl by methods analogous to those described for Intermediate 13.

| Int. | Name | Structure | Characterization | Aryl |
|---|---|---|---|---|
| 14 | 2-ethyl-4- fluoroaniline | 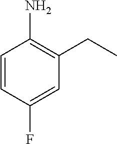 | MS (m/z) 140.1 (M + H)⁺. | 2-ethyl-4-fluoro- 1-nitrobenzene |
| 15 | 2-ethyl-3,4- difluoroaniline | 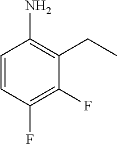 | MS (m/z) 158.0 (M + H)⁺. | 3,4-difluoro-2- vinylaniline |

### Intermediate 16

### 5-Fluoro-6-methoxy-2-methylpyridin-3-amine

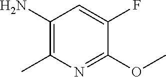

To a solution of N-(5-fluoro-6-methoxy-2-methylpyridin-3-yl)-1,1-diphenylmethanimine (24 g, 74.9 mmol) in 1,4-Dioxane (120 mL) at RT was added HCl in water (1.5 M, 200 mL, 300 mmol) and the reaction mixture was stirred at RT for an hour. The reaction mixture, combined with another reaction carried out on 25 g scale of N-(5-fluoro-6-methoxy-2-methylpyridin-3-yl)-1,1-diphenylmethanimine, was diluted with ice-water (50 mL) and extracted with DCM (80 mL×3). The combined organic extracts were neutralized with solid NaHCO₃ slowly until pH=8 and extracted with DCM (150 mL×3). The combined organic extracts were dried over Na₂SO₄ and concentrated under reduced pressure to give the title compound as a pale-yellow solid (20 g, 126 mmol, 168% yield). MS (m/z) 157.2 (M+H)⁺.

### Intermediate 17

### Ethyl 4-((4-fluoro-2-methylphenyl)amino)-6-(trifluoromethyl)nicotinate

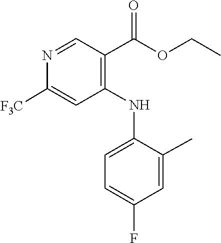 [SMILES: CCOC(=O)c1cnc(C(F)(F)F)cc1Nc1ccc(F)cc1C]

A solution of ethyl 4-bromo-6-(trifluoromethyl)nicotinate (1.1 g, 3.69 mmol), 4-fluoro-2-methylaniline (0.693 g, 5.54 mmol) and cesium carbonate (2.405 g, 7.38 mmol) in 1,4-dioxane (15 mL) was purged with N₂ for 15 minutes before BINAP (0.230 g, 0.369 mmol) and Pd₂(dba)₃ (0.169 g, 0.185 mmol) were added. The reaction mixture was purged with N₂ for 5 minutes and stirred at 90° C. for 15 h. The reaction was cooled and filtered through a pad of Celite® washing with EtOAc (80 mL). The filtrate was concentrated and purified by column chromatography (Biotage Isolera, 50 g Si₂O column, 0-20% EtOAc/petroleum ether over 1 h) to give the title compound as a pale yellow solid (850 mg, 2.128 mmol, 57.7% yield). MS (m/z) 343.1 (M+H)⁺.

Intermediates 18-32 were prepared from the indicated aryl halogen and aniline by methods analogous to those described for Intermediate 17.

| | | | | Aryl | |
|---|---|---|---|---|---|
| Int. | Name | Structure | Characterization | halogen | Aniline |
| 18 | methyl 5-((4- fluoro-2-iso- propylphenyl)- amino)-2-(tri- fluoromethyl)- isonicotinate | 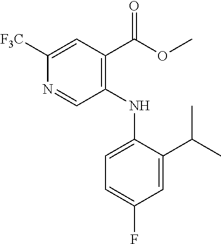 [SMILES: COC(=O)c1cc(C(F)(F)F)ncc1Nc1ccc(F)cc1C(C)C] | MS (m/z) 357.1 (M + H)⁺ | methyl 5- bromo-2- (trifluoro- methyl)iso- nicotinate | 4-fluoro-2- isopropyl- aniline |
| 19, 32 | methyl 5-((4- fluoro-2- methylphen- yl)amino)-2- (trifluoro- methyl)iso- nicotinate | 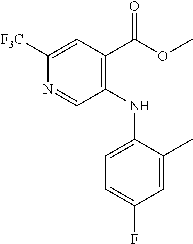 [SMILES: COC(=O)c1cc(C(F)(F)F)ncc1Nc1ccc(F)cc1C] | MS (m/z) 329.0 (M + H)⁺ | methyl 5- bromo-2- (trifluoro- methyl)iso- nicotinate | 4-fluoro-2- methylaniline |
| 20 | methyl 3-((4- fluoro-2- methylphen- yl)amino)iso- nicotinate | 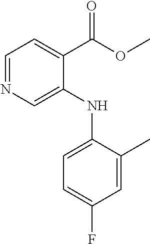 [SMILES: COC(=O)c1ccncc1Nc1ccc(F)cc1C] | MS (m/z) 261.3 (M + H)⁺ | methyl 3- bromoiso- nicotinate | 4-fluoro-2- methylaniline |
| 22 | methyl 5-((2- ethyl-4-fluoro- phenyl)amino)- 2-(trifluoro- methyl)iso- nicotinate | 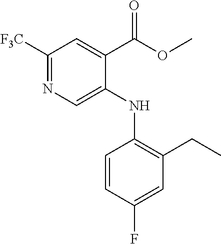 [SMILES: CCc1cc(F)ccc1Nc1cnc(C(F)(F)F)cc1C(=O)OC] | MS (m/z) 343.1 (M + H)⁺ | methyl 5- bromo-2- (trifluoro- methyl)iso- nicotinate | 2-ethyl-4- fluoroaniline |
| 23 | methyl 2- chloro-5-((4- fluoro-2- methylphen- yl)amino)iso- nicotinate | 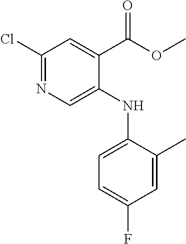 [SMILES: COC(=O)c1cc(Cl)ncc1Nc1ccc(F)cc1C] | MS (m/z) 295.2 (M + H)⁺ | methyl 5- bromo-2- chloroiso- nicotinate | 4-fluoro-2- methylaniline |
| 24, 31 | methyl 5-((2- methyl-4-(tri- fluorometh- oxy)phenyl)- amino)-2-(tri- fluoromethyl)- isonicotinate | 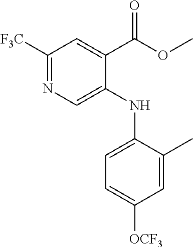 [SMILES: COC(=O)c1cc(C(F)(F)F)ncc1Nc1ccc(OC(F)(F)F)cc1C] | MS (m/z) 395.3 (M + H)⁺ | methyl 5- bromo-2- (trifluoro- methyl)iso- nicotinate | 2-methyl-4- (trifluoro- methoxy)- aniline |
| 25 | methyl 5- ((3,4-difluoro- 2-methyl-phen- yl)amino)-2- (trifluorometh- yl)isonicotinate | 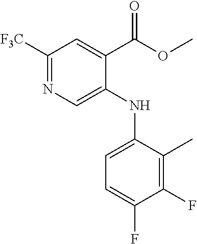 [SMILES: COC(=O)c1cc(C(F)(F)F)ncc1Nc1ccc(F)c(F)c1C] | MS (m/z) 347.2 (M + H)⁺ | methyl 5- bromo-2- (trifluoro- methyl)iso- nicotinate | 3,4-difluoro- 2-methyl- aniline |
| 26 | methyl 6- chloro-4-((4- fluoro-2- methylphen- yl)amino)- nicotinate | 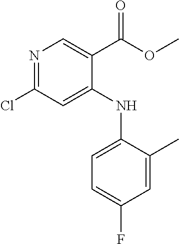 [SMILES: COC(=O)c1cnc(Cl)cc1Nc1ccc(F)cc1C] | MS (m/z) 295.2 (M + H)⁺ | methyl 4- bromo-6- chloro- nicotinate | 4-fluoro-2- methylaniline |
| 27 | ethyl 5-((2- ethyl-3,4- difluorophen- yl)amino)-2- (trifluoro- methyl)iso- nicotinate | 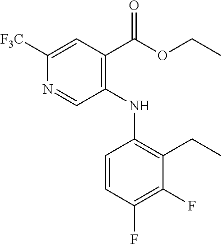 [SMILES: CCOC(=O)c1cc(C(F)(F)F)ncc1Nc1ccc(F)c(F)c1CC] | MS (m/z) 375.0 (M + H)⁺ | ethyl 5- bromo-2- (trifluoro- methyl)iso- nicotinate | 2-ethyl-3,4- difluoro- aniline |
| 28 | ethyl 4-((4- fluoro-2- methylphen- yl)amino)-2- (trifluoro- methyl)pyr- imidine-5- carboxylate | 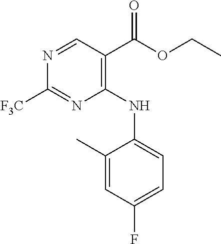 [SMILES: CCOC(=O)c1cnc(C(F)(F)F)nc1Nc1ccc(F)cc1C] | MS (m/z) 344.0 (M + H)⁺ | ethyl 4- chloro-2- (trifluoro- methyl)- pyrimidine- 5-carbox- ylate | 4-fluoro-2- methylaniline |
| 29 | methyl 2- chloro-5-((4- fluoro-2-iso- propylphen- yl)amino)iso- nicotinate | 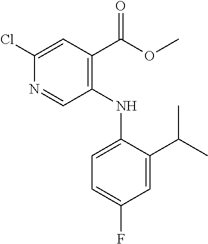 [SMILES: COC(=O)c1cc(Cl)ncc1Nc1ccc(F)cc1C(C)C] | MS (m/z) 323.0 (M + H)⁺ | methyl 5- bromo-2- chloroiso- nicotinate | 4-fluoro-2- isopropyl- aniline |
| 30 | ethyl 3-((4- fluoro-2-iso- propylphen- yl)amino)-6- methoxy- picolinate | 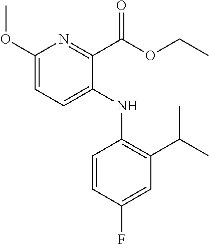 [SMILES: CCOC(=O)c1nc(OC)ccc1Nc1ccc(F)cc1C(C)C] | MS (m/z) 333.0 (M + H)⁺ | ethyl 3- bromo-6- methoxy- picolinate | 4-fluoro-2- isopropyl- aniline |

### Intermediate 33

### Ethyl 3-((4-fluoro-2-methylphenyl)amino)-5-(trifluoromethyl)pyrazine-2-carboxylate

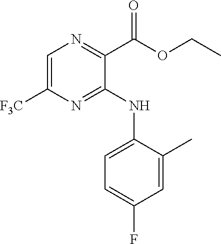 [SMILES: CCOC(=O)c1ncc(C(F)(F)F)nc1Nc1ccc(F)cc1C]

To a solution of ethyl 3-amino-5-(trifluoromethyl)pyrazine-2-carboxylate (700 mg, 2.98 mmol) in toluene (20 mL), 4-fluoro-1-iodo-2-methylbenzene (1054 mg, 4.46 mmol) and cesium carbonate (1455 mg, 4.46 mmol) were added at RT. The resultant reaction mixture was purged with N₂ for 10 min before Pd₂(dba)₃ (68.1 mg, 0.074 mmol) and (9,9-dimethyl-9H-xanthene-4,5-diyl)bis(diphenylphosphane) (86 mg, 0.149 mmol) were added. The reaction mixture was stirred at 90° C. in sealed tube for 16 h. Ice water (20 mL) was added and the reaction was extracted with EtOAc (2×50 mL). The combined organic extracts were washed with brine (10 mL), dried over Na₂SO₄, filtered and evaporated undervacuum. The crude product was absorbed on silica (1 g) in DCM (10 mL) and purified by flash chromatography (Isolera, 50 g Si2O column, 4% EtOAc/petroleum ether) to give the title compound as a yellow solid (720 mg, 1.909 mmol, 64.1% yield). MS (m/z) 344.0 (M+H)⁺.

Intermediate 34 was prepared from the indicated aryl halogen and aniline by methods analogous to those described for Intermediate 33.

| Int. | Name | Structure | Characterization | Aryl halogen | Aniline |
|---|---|---|---|---|---|
| 34 | ethyl 3-((4- fluoro-2- methylphen- yl)amino)-6- (trifluoro- methyl)pyr- azine-2- carboxylate | 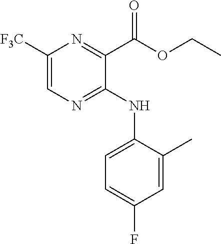 [SMILES: CCOC(=O)c1nc(C(F)(F)F)cnc1Nc1ccc(F)cc1C] | MS (m/z) 344.0 (M + H)⁺ | 4-fluoro-1- iodo-2-meth- ylbenzene | ethyl 3- amino-6- (trifluoro- methyl)pyr- azine-2- carboxylate |

### Intermediate 35

### Ethyl 3-((4-fluoro-2-isopropylphenyl)amino)-6-hydroxypicolinate

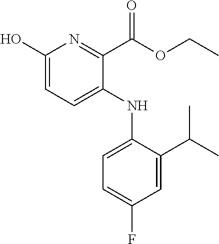 [SMILES: CCOC(=O)c1nc(O)ccc1Nc1ccc(F)cc1C(C)C]

To a stirring solution of ethyl 3-((4-fluoro-2-isopropylphenyl)amino)-6-methoxypicolinate (0.7 g, 2.106 mmol) in acetonitrile (10 mL) at 0° C. under N₂ was added iodotrimethylsilane (0.717 mL, 5.27 mmol) and reaction mixture was stirred at 80° C. for 1 h. The reaction mixture was allowed to cool to 30° C. and concentrated under reduced pressure. The residue was dissolved in EtOAc (50 mL) and washed with saturated sodium thiosulphate (50 mL). The organic phase was dried over Na₂SO₄ and concentrated in vacuo to give the title compound as a pale-yellow solid (700 mg, 1.690 mmol, 80% yield). MS (m/z) 319.1 (M+H)⁺.

### Intermediate 36

### 3-((4-Fluoro-2-methylphenyl)amino)-5-(trifluoromethyl)picolinic acid

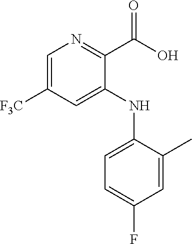 [SMILES: Cc1cc(F)ccc1Nc1cc(C(F)(F)F)cnc1C(=O)O]

To a stirring solution of methyl 3-chloro-5-(trifluoromethyl)picolinate (1.5 g, 6.26 mmol) in 1,4-Dioxane (20 mL) were added 4-fluoro-2-methylaniline (1.567 g, 12.52 mmol), BINAP (0.390 g, 0.626 mmol), Cs₂CO₃ (4.08 g, 12.52 mmol) and Pd₂dba₂ (0.287 g, 0.313 mmol). The reaction was purged with N₂ for 5 minutes and then stirred at 100° C. for 12 h. After the reaction was cooled, water (50 mL) was added and the reaction was washed with EtOAc (25 mL). The aqueous layer was acidified with 1.5 N HCl (5 mL) and extracted with EtOAc (75 mL). The organic layer was dried over Na₂SO₄ and concentrated under reduced pressure to give the title compound as a yellow solid (2 g, 4.71 mmol, 75% yield). MS (m/z) 314.9 (M+H)⁺.

### Intermediate 37

### 4-((4-Fluoro-2-methylphenyl)amino)-2-(trifluoromethyl)pyrimidine-5-carboxylic acid

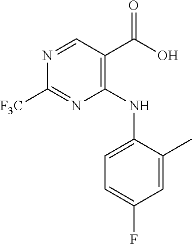 [SMILES: Cc1cc(F)ccc1Nc1nc(C(F)(F)F)ncc1C(=O)O]

To a stirring solution of ethyl 4-((4-fluoro-2-methylphenyl)amino)-2-(trifluoromethyl)pyrimidine-5-carboxylate (6 g, 17.48 mmol) in THF (40.0 mL) was added a solution of LiOH (4.40 g, 105 mmol) in water (40.0 mL) dropwise over 5 minutes at RT. The resulting reaction mixture was stirred at 60° C. for 16 h. The reaction mixture was allowed to cool to RT and concentrated. The residue was acidified with 1.5 N HCl (100 mL) until pH 3-4. The yellow solid precipitate was collected by filtration and dried under reduced pressure to give the title compound as a yellow solid (4.5 g, 14.10 mmol, 81% yield). MS (m/z) 316.0 (M+H)⁺.

Intermediates 38-54 were prepared from the indicated ester by methods analogous to those described for Intermediate 37.

| Int. | Name | Structure | Characterization | Ester |
|---|---|---|---|---|
| 38 | 2-chloro-5-((4- fluoro-2-iso- propylphenyl)- amino)iso- nicotinic acid | 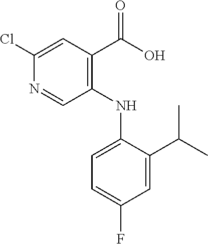 [SMILES: CC(C)c1cc(F)ccc1Nc1cnc(Cl)cc1C(=O)O] | MS (m/z) 309.0 (M + H)⁺ | methyl 2-chloro- 5-((4-fluoro-2- isopropylphenyl)- amino)isonicotinate |
| 39 | 4-((4-fluoro-2- methylphenyl)- amino)-6-(tri- fluoromethyl)- nicotinic acid | 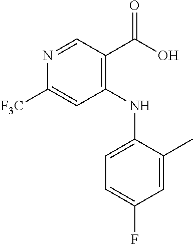 [SMILES: Cc1cc(F)ccc1Nc1cc(C(F)(F)F)ncc1C(=O)O] | MS (m/z) 315.0 (M + H)⁺ | ethyl 4-((4-fluoro- 2-methylphenyl)- amino)-6-(trifluoro- methyl)nicotinate |
| 40 | 5-((4-fluoro-2- isopropylphen- yl)amino)-2- (trifluorometh- yl)isonicotinic acid | 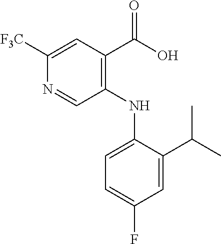 [SMILES: CC(C)c1cc(F)ccc1Nc1cnc(C(F)(F)F)cc1C(=O)O] | MS (m/z) 343.0 (M + H)⁺ | methyl 5-((4-fluoro- 2-isopropylphen- yl)amino)-2-(tri- fluoromethyl)iso- nicotinate |
| 41, 54 | 5-((4-fluoro-2- methylphenyl)- amino)-2-(tri- fluoromethyl)- isonicotinic acid | 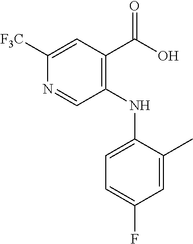 [SMILES: Cc1cc(F)ccc1Nc1cnc(C(F)(F)F)cc1C(=O)O] | MS (m/z) 315.0 (M + H)⁺ | methyl 5-((4-fluoro- 2-methylphenyl)- amino)-2-(trifluoro- methyl)isonicotinate |
| 42 | 3-((4-fluoro- 2-methylphen- yl)amino)iso- nicotinic acid | 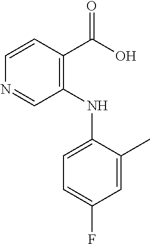 [SMILES: Cc1cc(F)ccc1Nc1cnccc1C(=O)O] | MS (m/z) 247.1 (M + H)⁺ | methyl 3-((4-fluoro- 2-methylphenyl)- amino)isonicotinate |
| 44 | 5-((2-ethyl-4- fluorophenyl)- amino)-2-(tri- fluoromethyl)- isonicotinic acid | 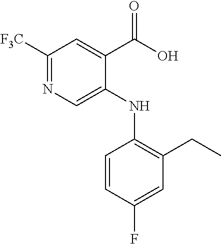 [SMILES: CCc1cc(F)ccc1Nc1cnc(C(F)(F)F)cc1C(=O)O] | MS (m/z) 329.1 (M + H)⁺ | methyl 5-((2-ethyl- 4-fluorophenyl)- amino)-2-(trifluoro- methyl)isonicotinate |
| 45 | 3-((4-fluoro-2- methylphenyl)- amino)-5-(tri- fluoromethyl)- pyrazine-2- carboxylic acid | 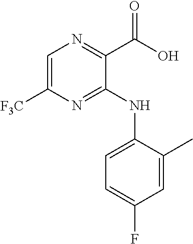 [SMILES: Cc1cc(F)ccc1Nc1nc(C(F)(F)F)cnc1C(=O)O] | MS (m/z) 316.1 (M + H)⁺ | ethyl 3-((4-fluoro- 2-methylphenyl)- amino)-5-(trifluoro- methyl)pyrazine-2- carboxylate |
| 46 | 3-((4-fluoro-2- methylphenyl)- amino)-6-(tri- fluoromethyl)- pyrazine-2- carboxylic acid | 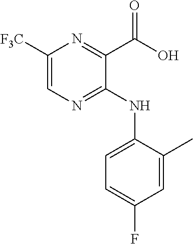 [SMILES: Cc1cc(F)ccc1Nc1ncc(C(F)(F)F)nc1C(=O)O] | MS (m/z) 316.1 (M + H)⁺ | ethyl 3-((4-fluoro- 2-methylphenyl)- amino)-6-(trifluoro- methyl)pyrazine-2- carboxylate |
| 47 | 2-chloro-5-((4- fluoro-2-meth- ylphenyl)- amino)isonico- tinic acid | 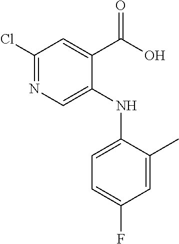 [SMILES: Cc1cc(F)ccc1Nc1cnc(Cl)cc1C(=O)O] | MS (m/z) 281.1 (M + H)⁺ | methyl 2-chloro-5- ((4-fluoro-2-meth- ylphenyl)amino)- isonicotinate |
| 48, 53 | 5-((2-methyl- 4-(trifluoro- methoxy)phen- yl)amino)-2- (trifluorometh- yl)isonicotinic acid | 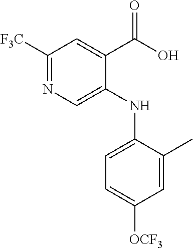 [SMILES: Cc1cc(OC(F)(F)F)ccc1Nc1cnc(C(F)(F)F)cc1C(=O)O] | MS (m/z) 381.2 (M + H)⁺ | methyl 5-((2-meth- yl-4-(trifluorometh- oxy)phenyl)amino)- 2-(trifluoromethyl) isonicotinate |
| 49 | 6-chloro-4- ((4-fluoro-2- methylphenyl)- amino)nicotinic acid | 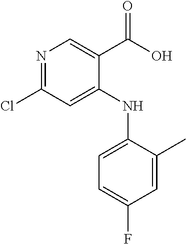 [SMILES: Cc1cc(F)ccc1Nc1cc(Cl)ncc1C(=O)O] | MS (m/z) 281.2 (M + H)⁺ | methyl 6-chloro-4- ((4-fluoro-2-meth- ylphenyl)amino)- nicotinate |
| 50 | 5-((3,4- difluoro-2- methylphen- yl)amino)-2- (trifluorometh- yl)isonicotinic acid | 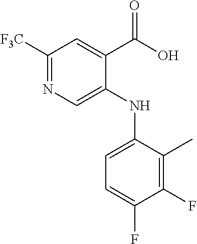 [SMILES: Cc1c(Nc2cnc(C(F)(F)F)cc2C(=O)O)ccc(F)c1F] | MS (m/z) 333.1 (M + H)⁺ | methyl 5-((3,4- difluoro-2-methyl- phenyl)amino)-2- (trifluoromethyl) isonicotinate |
| 51 | 3-((4-fluoro-2- isopropylphen- yl)amino)-6- hydroxy- picolinic acid | 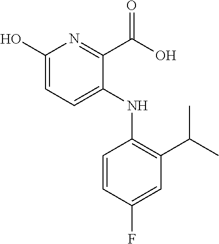 [SMILES: CC(C)c1cc(F)ccc1Nc1ccc(O)nc1C(=O)O] | MS (m/z) 289.1 (M − H)⁻ | ethyl 3-((4-fluoro- 2-isopropylphenyl)- amino)-6-hydroxy- picolinate |
| 52 | 5-((2-ethyl- 3,4-difluoro- phenyl)amino)- 2-(trifluoro- methyl)iso- nicotinic acid | 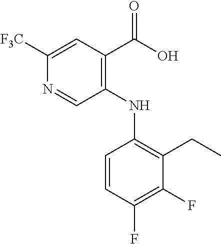 [SMILES: CCc1c(Nc2cnc(C(F)(F)F)cc2C(=O)O)ccc(F)c1F] | MS (m/z) 347.1 (M + H)⁺ | ethyl 5-((2-ethyl- 3,4-difluorophenyl)- amino)-2-(trifluoro- methyl)isonicotinate |

### Intermediate 55

### 4-((2-Ethyl-4-fluorophenyl)amino)-6-(trifluoromethyl)nicotinic acid

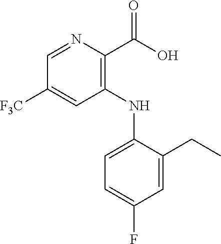 [SMILES: CCc1cc(F)ccc1Nc1cc(C(F)(F)F)cnc1C(=O)O]

A solution of 4-chloro-6-(trifluoromethyl)nicotinic acid (0.338 g, 1.499 mmol) and 2-ethyl-4-fluoroaniline (0.292 g, 2.098 mmol) in acetic acid (3.75 ml) was stirred at 100° C. for 18 hr. The reaction was cooled, water was added and the solid precipitate was collected by filtration, washed with water, and air dried to give the title compound as a grey solid (326 mg, 0.993 mmol, 66.3% yield). MS (m/z) 329.2 (M+H)⁺.

### Intermediate 56

### 3-((4-Fluoro-2-methylphenyl)amino)-N-(6-methoxy-2-methylpyridin-3-yl)-5-(trifluoromethyl)picolinamide

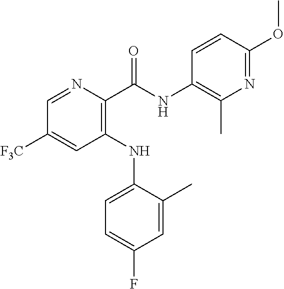 [SMILES: COc1ccc(NC(=O)c2ncc(C(F)(F)F)cc2Nc2ccc(F)cc2C)c(C)n1]

To a stirring solution of 3-((4-fluoro-2-methylphenyl)amino)-5-(trifluoromethyl)picolinic acid (2 g, 6.36 mmol) in DMF (20 mL) were added 6-methoxy-2-methylpyridin-3-amine (0.879 g, 6.36 mmol), HATU (2.420 g, 6.36 mmol), and TEA (0.887 mL, 6.36 mmol). After stirring at 25° C. for 1 h, cold water (50 mL) was added and the reaction was stirred for 1 h. The solid precipitate was collected by filtration and washed with Et₂O (5 mL) to give the title compound as a yellow solid (1.1 g, 2.431 mmol, 38.2% yield). MS (m/z) 435.0 (M+H)⁺.

Intermediates 57-73 were prepared from the indicated amine and carboxylic acid by methods analogous to those described for Intermediate 56.

| Int. | Name | Structure | Characterization | Amine | Acid |
|---|---|---|---|---|---|
| 57 | 2-chloro-5-((4- fluoro-2-iso- propylphenyl)- amino)-N-(6- methoxy-2- methylpyridin- 3-yl)isonicotin- amide | 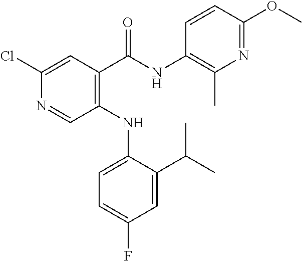 [SMILES: COc1ccc(NC(=O)c2cc(Cl)ncc2Nc2ccc(F)cc2C(C)C)c(C)n1] | MS (m/z) 429.2 (M + H)⁺ | 6-methoxy-2- methylpyridin- 3-amine | 2-chloro-5-((4- fluoro-2-iso- propylphenyl)- amino)isonico- tinic acid |
| 58 | 4-((4-fluoro-2- methylphenyl)- amino)-N-(6- methoxy-2- methylpyridin- 3-yl)-6-(tri- fluoromethyl)- nicotinamide | 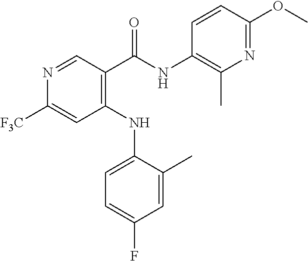 [SMILES: COc1ccc(NC(=O)c2cnc(C(F)(F)F)cc2Nc2ccc(F)cc2C)c(C)n1] | MS (m/z) 435.0 (M + H)⁺ | 6-methoxy-2- methylpyridin- 3-amine | 4-((4-fluoro-2- methylphenyl)- amino)-6-(tri- fluoromethyl) nicotinic acid |
| 69 | 3-((4-fluoro-2- isopropylphen- yl)amino)-6- hydroxy-N-(6- methoxy-2- methylpyridin- 3-yl)picolin- amide | 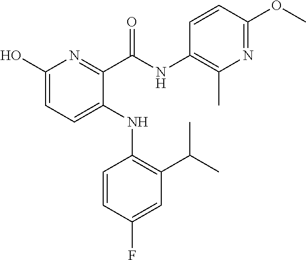 [SMILES: COc1ccc(NC(=O)c2nc(O)ccc2Nc2ccc(F)cc2C(C)C)c(C)n1] | MS (m/z) 411.1 (M + H)⁺ | 6-methoxy-2- methylpyridin- 3-amine | 3-((4-fluoro-2- isopropylphen- yl)amino)-6- hydroxypico- linic acid |
| 60 | 3-((4-fluoro-2- methylphenyl)- amino)-N-(6- methoxy-2- methylpyridin- 3-yl)isonicotin- amide | 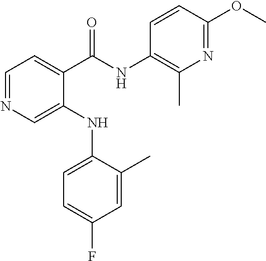 [SMILES: COc1ccc(NC(=O)c2ccncc2Nc2ccc(F)cc2C)c(C)n1] | MS (m/z) 367.3 (M + H)⁺ | 6-methoxy-2- methylpyridin- 3-amine | 3-((4-fluoro-2- methylphenyl)- amino)isonico- tinic acid |
| 62 | 5-((2-ethyl-4- fluorophenyl)- amino)-N-(6- methoxy-2- methylpyridin- 3-yl)-2-(tri- fluoromethyl)- isonicotinamide | 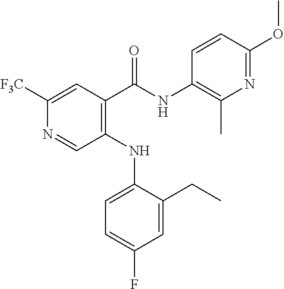 [SMILES: CCc1cc(F)ccc1Nc1cnc(C(F)(F)F)cc1C(=O)Nc1ccc(OC)nc1C] | MS (m/z) 449.2 (M + H)⁺ | 6-methoxy-2- methylpyridin- 3-amine | 5-((2-ethyl-4- fluorophenyl)- amino)-2-(tri- fluoromethyl) isonicotinic acid |
| 63 | 3-((4-fluoro-2- methylphenyl)- amino)-N-(6- methoxy-2- methylpyridin- 3-yl)-5-(tri- fluoromethyl)- pyrazine-2- carboxamide | 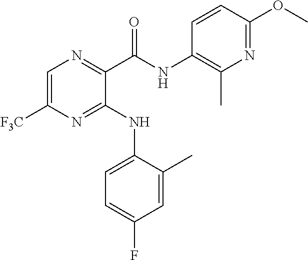 [SMILES: COc1ccc(NC(=O)c2ncc(C(F)(F)F)nc2Nc2ccc(F)cc2C)c(C)n1] | MS (m/z) 436.0 (M + H)⁺ | 6-methoxy-2- methylpyridin- 3-amine | 3-((4-fluoro-2- methylphenyl)- amino)-5-(tri- fluoromethyl) pyrazine-2- carboxylic acid |
| 64 | 3-((4-fluoro-2- methylphenyl)- amino)-N-(6- methoxy-2- methylpyridin- 3-yl)-6-(tri- fluoromethyl)- pyrazine-2- carboxamide | 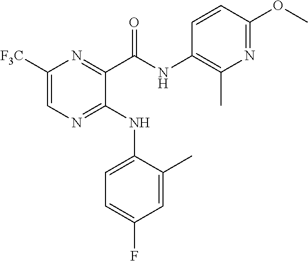 [SMILES: COc1ccc(NC(=O)c2nc(C(F)(F)F)cnc2Nc2ccc(F)cc2C)c(C)n1] | MS (m/z) 436.0 (M + H)⁺ | 6-methoxy-2- methylpyridin- 3-amine | 3-((4-fluoro-2- methylphenyl)- amino)-6-(tri- fluoromethyl) pyrazine-2- carboxylic acid |
| 65 | 2-chloro-5-((4- fluoro-2-meth- ylphenyl)- amino)-N-(6- methoxy-2- methylpyridin- 3-yl)isonico- tinamide | 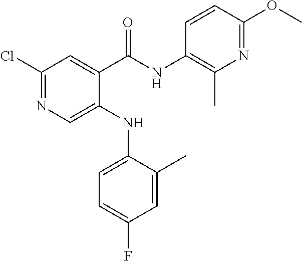 [SMILES: COc1ccc(NC(=O)c2cc(Cl)ncc2Nc2ccc(F)cc2C)c(C)n1] | MS (m/z) 401.3 (M + H)⁺ | 6-methoxy-2- methylpyridin- 3-amine | 2-chloro-5-((4- fluoro-2-meth- ylphenyl)- amino)isonico- tinic acid |
| 66 | N-(6-methoxy- 2-methylpyr- idin-3-yl)-5- ((2-methyl-4- (trifluorometh- oxy)phenyl)- amino)-2-(tri- fluoromethyl)- isonicotinamide | 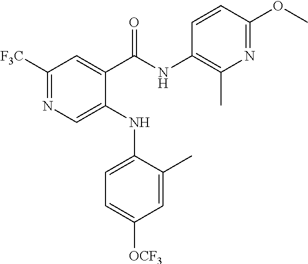 [SMILES: COc1ccc(NC(=O)c2cc(C(F)(F)F)ncc2Nc2ccc(OC(F)(F)F)cc2C)c(C)n1] | MS (m/z) 501.3 (M + H)⁺ | 6-methoxy-2- methylpyridin- 3-amine | 5-((2-methyl-4- (trifluorometh- oxy)phenyl)- amino)-2-(tri- fluoromethyl) isonicotinic acid |
| 67 | 5-((3,4-difluoro- 2-methylphen- yl)amino)-N-(6- methoxy-2- methylpyridin- 3-yl)-2-(tri- fluoromethyl)- isonicotinamide | 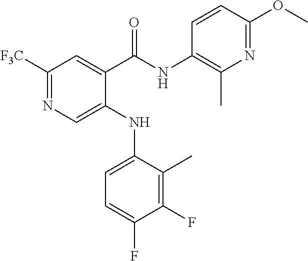 [SMILES: COc1ccc(NC(=O)c2cc(C(F)(F)F)ncc2Nc2ccc(F)c(F)c2C)c(C)n1] | MS (m/z) 453.3 (M + H)⁺ | 6-methoxy-2- methylpyridin- 3-amine | 5-((3,4-difluoro- 2-methylphen- yl)amino)-2- (trifluoromethyl) isonicotinic acid |
| 68 | 6-chloro-4-((4- fluoro-2-meth- ylphenyl)- amino)-N-(6- methoxy-2- methylpyridin- 3-yl)nicotin- amide | 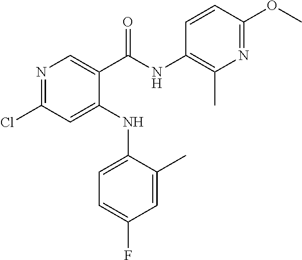 [SMILES: COc1ccc(NC(=O)c2cnc(Cl)cc2Nc2ccc(F)cc2C)c(C)n1] | MS (m/z) 401.2 (M + H)⁺ | 6-methoxy-2- methylpyridin- 3-amine | 6-chloro-4-((4- fluoro-2-meth- ylphenyl)- amino)nicotinic acid |
| 69 | 4-((2-ethyl-4- fluorophenyl)- amino)-N-(6- methoxy-2- methylpyridin- 3-yl)-6-(tri- fluoromethyl)- nicotinamide | 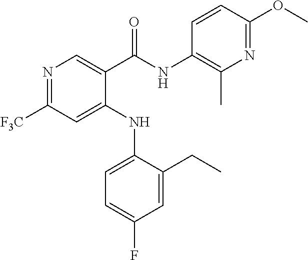 [SMILES: CCc1cc(F)ccc1Nc1cc(C(F)(F)F)ncc1C(=O)Nc1ccc(OC)nc1C] | MS (m/z) 449.4 (M + H)⁺ | 6-methoxy-2- methylpyridin- 3-amine | 4-((2-ethyl-4- fluorophenyl)- amino)-6-(tri- fluoromethyl) nicotinic acid |
| 70 | 5-((2-ethyl-3,4- difluorophen- yl)amino)-N- (6-methoxy-2- methylpyridin- 3-yl)-2-(tri- fluoromethyl)- isonicotinamide | 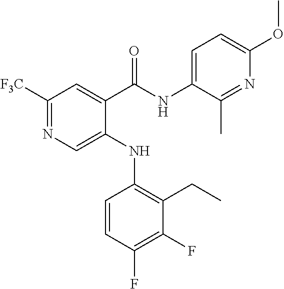 [SMILES: CCc1c(Nc2cnc(C(F)(F)F)cc2C(=O)Nc2ccc(OC)nc2C)ccc(F)c1F] | MS (m/z) 467.2 (M + H)⁺ | 6-methoxy-2- methylpyridin- 3-amine | 5-((2-ethyl-3,4- difluorophenyl)- amino)-2-(tri- fluoromethyl) isonicotinic acid |
| 71 | N-(5-fluoro-6- methoxy-2- methylpyridin- 3-yl)-5-((2- methyl-4-(tri- fluoromethoxy)- phenyl)amino)- 2-(trifluoro- methyl)iso- nicotinamide | 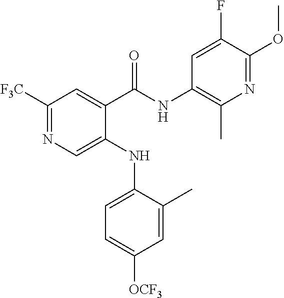 [SMILES: COc1nc(C)c(NC(=O)c2cc(C(F)(F)F)ncc2Nc2ccc(OC(F)(F)F)cc2C)cc1F] | MS (m/z) 519.1 (M + H)⁺ | 5-fluoro-6- methoxy-2- methylpyridin- 3-amine | 5-((2-methyl-4- (trifluorometh- oxy)phenyl)- amino)-2-(tri- fluoromethyl) isonicotinic acid |
| 72 | 5-((4-fluoro-2- methylphenyl)- amino)-N-(5- fluoro-6-meth- oxy-2-methyl- pyridin-3-yl)-2- (trifluorometh- yl)isonicotin- amide | 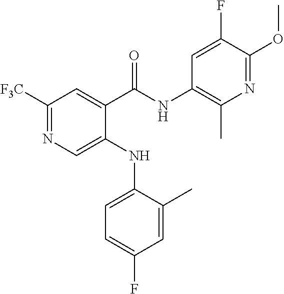 [SMILES: COc1nc(C)c(NC(=O)c2cc(C(F)(F)F)ncc2Nc2ccc(F)cc2C)cc1F] | MS (m/z) 453.2 (M + H)⁺ | 5-fluoro-6- methoxy-2- methylpyridin- 3-amine | 5-((4-fluoro-2- methylphenyl)- amino)-2-(tri- fluoromethyl) isonicotinic acid |
| 73 | 5-((2-methyl- 4-(trifluoro- methoxy)phen- yl)amino)-N- (3-methylpyr- idin-4-yl)-2- (trifluorometh- yl)isonicotin- amide | 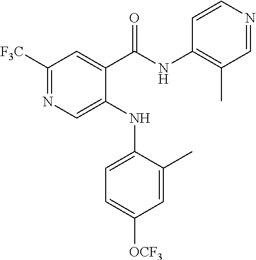 [SMILES: Cc1cnccc1NC(=O)c1cc(C(F)(F)F)ncc1Nc1ccc(OC(F)(F)F)cc1C] | MS (m/z) 471.3 (M + H)⁺ | 3-methylpyr- idin-4-amine | 5-((2-methyl-4- (trifluorometh- oxy)phenyl)- amino)-2-(tri- fluoromethyl) isonicotinic acid |

### Intermediate 74

### 4-((4-Fluoro-2-methylphenyl)amino)-N-(6-methoxy-2-methylpyridin-3-yl)-2-(trifluoromethyl)pyrimidine-5-carboxamide

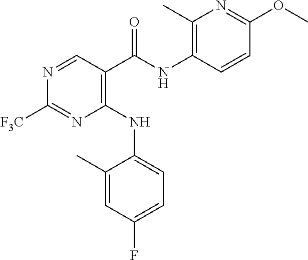 [SMILES: COc1ccc(NC(=O)c2cnc(C(F)(F)F)nc2Nc2ccc(F)cc2C)c(C)n1]

A solution of 4-((4-fluoro-2-methylphenyl)amino)-2-(trifluoromethyl)pyrimidine-5-carboxylic acid (1.5 g, 4.76 mmol) and thionyl chloride (15 mL, 206 mmol) was stirred under N₂ at 80° C. for 1.5 h. The reaction mixture was allowed to cool to RT and concentrated under reduced pressure. The obtained yellow solid residue was dissolved in DCM (10 mL) and cooled to 0° C. To the reaction solution was added a solution of triethylamine (1.990 mL, 14.28 mmol) and 6-methoxy-2-methylpyridin-3-amine (0.789 g, 5.71 mmol) in DCM (5 mL) dropwise over 5 minutes under N₂. After stirring under N₂ at RT for 2 h, the reaction mixture was cooled to 0° C., quenched with water (25 mL), and extracted with DCM (3×50 mL). The combined organic extracts were washed with water (50 mL), brine (50 mL) and saturated NaHCO₃ (25 mL), dried over Na₂SO₄ and concentrated in vacuo. The residue was purified by column chromatography (Biotage, 50 g Si₂O SNAP column, 0-50% EtOAc/petroleum ether over 50 minutes) to give the title compound as a yellow solid (1.7 g, 3.86 mmol, 81% yield). MS (m/z) 436.1 (M+H)⁺.

Intermediates 75-76 were prepared from the indicated amine and carboxylic acid by methods analogous to those described for Intermediate 74.

| Int. | Name | Structure | Characterization | Amine | acid |
|---|---|---|---|---|---|
| 75 | 5-((4-fluoro-2- isopropylphenyl)- amino)-N-(6- methoxy-2-meth- ylpyridin-3-yl)-2- (trifluoromethyl)- isonicotinamide | 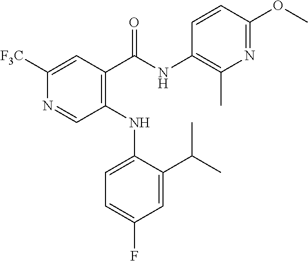 [SMILES: COc1ccc(NC(=O)c2cc(C(F)(F)F)ncc2Nc2ccc(F)cc2C(C)C)c(C)n1] | MS (m/z) 463.1 (M + H)⁺ | 6-methoxy-2- methylpyridin- 3-amine | 5-((4-fluoro-2- isopropylphen- yl)amino)-2- (trifluoromethyl) isonicotinic acid |
| 76 | 5-((4-fluoro-2- methylphenyl)- amino)-N-(6- methoxy-2- methylpyridin-3- yl)-2-(trifluoro- methyl)isonico- tinamide | 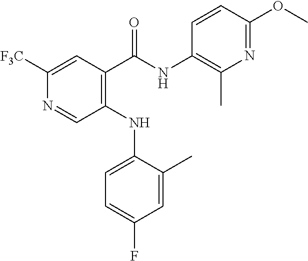 [SMILES: COc1ccc(NC(=O)c2cc(C(F)(F)F)ncc2Nc2ccc(F)cc2C)c(C)n1] | MS (m/z) 435.1 (M + H)⁺ | 6-methoxy-2- methylpyridin- 3-amine | 5-((4-fluoro-2- methylphenyl)- amino)-2-(tri- fluoromethyl) isonicotinic acid |

### Intermediate 77

### 1-(4-Fluoro-2-methylphenyl)-3-(6-methoxy-2-methylpyridin-3-yl)-7-(trifluoromethyl)-2,3-dihydropyrido[3,2-d]pyrimidin-4(1H)-one

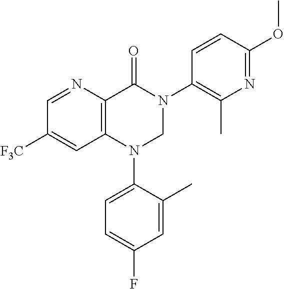 [SMILES: COc1ccc(N2CN(c3ccc(F)cc3C)c3cc(C(F)(F)F)cnc3C2=O)c(C)n1]

To a stirring solution of 3-((4-fluoro-2-methylphenyl)amino)-N-(6-methoxy-2-methylpyridin-3-yl)-5-(trifluoromethyl)picolinamide (1.1 g, 2.53 mmol) in acetonitrile (15 mL) were added Cs₂CO₃ (3.30 g, 10.13 mmol) and diiodomethane (2.035 g, 7.60 mmol) and the reaction mixture was stirred at 90° C. for 10 h. The reaction mixture was quenched with water (50 mL), extracted with DCM (50 mL), dried over Na₂SO₄ and concentrated under reduced pressure. The residue was purified by column chromatography (Biotage Isolera, 10 g Si₂O SNAP column, 2-9% EtOAc/petroleum ether over 35 minutes) to give the title compound as a yellow solid (430 mg, 0.954 mmol, 37.7% yield). MS (m/z) 447.0 (M+H)⁺.

Intermediates 78-96 were prepared from the indicated amide by methods analogous to those described for Intermediate 77.

| Int. | Name | Structure | Characterization | Amide |
|---|---|---|---|---|
| 78 | 1-(4-fluoro-2-meth- ylphenyl)-3-(6- methoxy-2-methyl- pyridin-3-yl)-7- (trifluoromethyl)- 2,3-dihydropyr- imido[4,5-d]pyr- imidin-4(1H)-one | 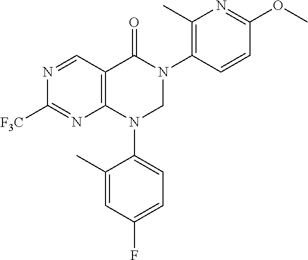 [SMILES: COc1ccc(N2CN(c3ccc(F)cc3C)c3nc(C(F)(F)F)ncc3C2=O)c(C)n1] | MS (m/z) 448.1 (M + H)⁺ | 4-((4-fluoro-2-meth- ylphenyl)amino)-N- (6-methoxy-2-meth- ylpyridin-3-yl)-2- (trifluoromethyl)- pyrimidine-5-carbox- amide |
| 79 | 6-chloro-1-(4- fluoro-2-isopropyl- phenyl)-3-(6-meth- oxy-2-methylpyr- idin-3-yl)-2,3- dihydropyrido[3,4- d]pyrimidin-4(1H)- one | 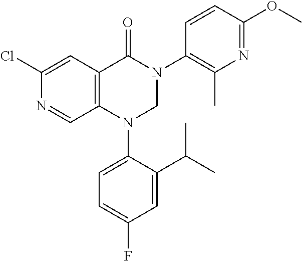 [SMILES: COc1ccc(N2CN(c3ccc(F)cc3C(C)C)c3cnc(Cl)cc3C2=O)c(C)n1] | MS (m/z) 441.2 (M + H)⁺ | 2-chloro-5-((4- fluoro-2-isopropyl- phenyl)amino)-N-(6- methoxy-2-methyl- pyridin-3-yl)iso- nicotinamide |
| 80 | 1-(4-fluoro-2-meth- ylphenyl)-3-(6- methoxy-2-meth- ylpyridin-3-yl)-7- (trifluoromethyl)- 2,3-dihydropyrido- [4,3-d]pyrimidin- 4(1H)-one | 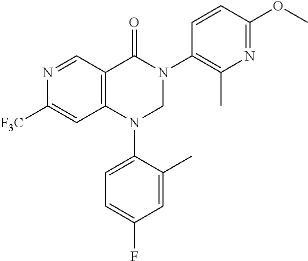 [SMILES: COc1ccc(N2CN(c3ccc(F)cc3C)c3cc(C(F)(F)F)ncc3C2=O)c(C)n1] | MS (m/z) 447.0 (M + H)⁺ | 4-((4-fluoro-2-meth- ylphenyl)amino)-N- (6-methoxy-2-meth- ylpyridin-3-yl)-6- (trifluoromethyl)- nicotinamide |
| 81 | 1-(4-fluoro-2-iso- propylphenyl)-3-(6- methoxy-2-methyl- pyridin-3-yl)-6- (trifluoromethyl)- 2,3-dihydropyrido- [3,4-d]pyrimidin- 4(1H)-one | 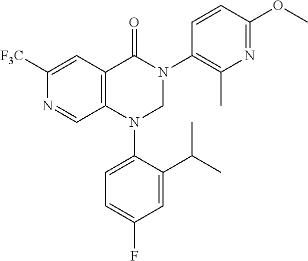 [SMILES: COc1ccc(N2CN(c3ccc(F)cc3C(C)C)c3cnc(C(F)(F)F)cc3C2=O)c(C)n1] | MS (m/z) 475.2 (M + H)⁺ | 5-((4-fluoro-2-iso- propylphenyl)- amino)-N-(6-meth- oxy-2-methylpyr- idin-3-yl)-2-(tri- fluoromethyl)iso- nicotinamide |
| 82 | 1-(4-fluoro-2-meth- ylphenyl)-3-(6- methoxy-2-meth- ylpyridin-3-yl)-6- (trifluoromethyl)- 2,3-dihydro- pyrido[3,4-d]pyri- midin-4(1H)-one | 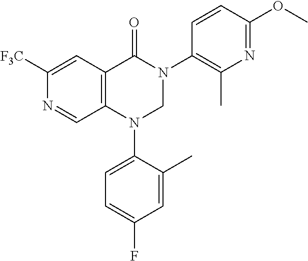 [SMILES: COc1ccc(N2CN(c3ccc(F)cc3C)c3cnc(C(F)(F)F)cc3C2=O)c(C)n1] | MS (m/z) 447.0 (M + H)⁺ | 5-((4-fluoro-2-meth- ylphenyl)amino)-N- (6-methoxy-2-meth- ylpyridin-3-yl)-2- (trifluoromethyl)iso- nicotinamide |
| 83 | 1-(4-fluoro-2-meth- ylphenyl)-3-(6- methoxy-2-meth- ylpyridin-3-yl)-2,3- dihydropyrido[3,4- d]pyrimidin-4(1H)- one | 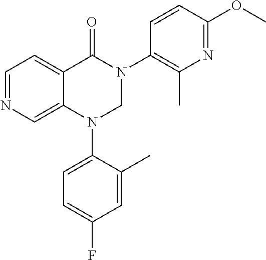 [SMILES: COc1ccc(N2CN(c3ccc(F)cc3C)c3cnccc3C2=O)c(C)n1] | MS (m/z) 379.4 (M + H)⁺ | 3-((4-fluoro-2-meth- ylphenyl)amino)-N- (6-methoxy-2-meth- ylpyridin-3-yl)iso- nicotinamide |
| 85 | 1-(2-ethyl-4-fluoro- phenyl)-3-(6-meth- oxy-2-methylpyr- idin-3-yl)-6-(tri- fluoromethyl)-2,3- dihydropyrido[3,4- d]pyrimidin-4(1H)- one | 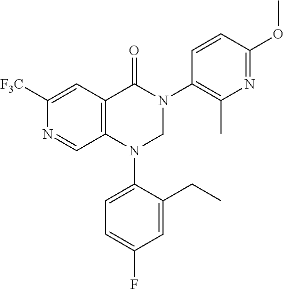 [SMILES: CCc1cc(F)ccc1N1CN(c2ccc(OC)nc2C)C(=O)c2cc(C(F)(F)F)ncc21] | MS (m/z) 461.2 (M + H)⁺ | 5-((2-ethyl-4-fluoro- phenyl)amino)-N- (6-methoxy-2-meth- ylpyridin-3-yl)-2- (trifluoromethyl)- isonicotinamide |
| 86 | 1-(4-fluoro-2-meth- ylphenyl)-3-(6- methoxy-2-methyl- pyridin-3-yl)-7- (trifluoromethyl)- 2,3-dihydropteridin- 4(1H)-one | 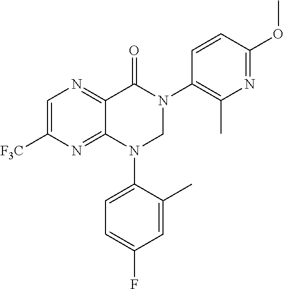 [SMILES: COc1ccc(N2CN(c3ccc(F)cc3C)c3nc(C(F)(F)F)cnc3C2=O)c(C)n1] | MS (m/z) 448.0 (M + H)⁺ | 3-((4-fluoro-2-meth- ylphenyl)amino)-N- (6-methoxy-2-meth- ylpyridin-3-yl)-5- (trifluoromethyl)- pyrazine-2-carbox- amide |
| 87 | 1-(4-fluoro-2-meth- ylphenyl)-3-(6- methoxy-2-meth- ylpyridin-3-yl)-6- (trifluoromethyl)- 2,3-dihydropteridin- 4(1H)-one | 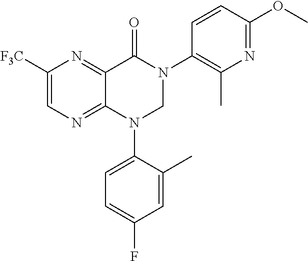 [SMILES: COc1ccc(N2CN(c3ccc(F)cc3C)c3ncc(C(F)(F)F)nc3C2=O)c(C)n1] | MS (m/z) 448.2 (M + H)⁺ | 3-((4-fluoro-2-meth- ylphenyl)amino)-N- (6-methoxy-2-meth- ylpyridin-3-yl)-6- (trifluoromethyl)- pyrazine-2-carbox- amide |
| 88 | 6-chloro-1-(4- fluoro-2-methyl- phenyl)-3-(6-meth- oxy-2-methylpyr- idin-3-yl)-2,3- dihydropyrido[3,4- d]pyrimidin-4(1H)- one | 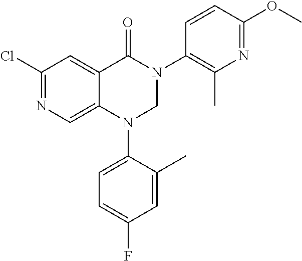 [SMILES: COc1ccc(N2CN(c3ccc(F)cc3C)c3cnc(Cl)cc3C2=O)c(C)n1] | MS (m/z) 413.3 (M + H)⁺ | 2-chloro-5-((4- fluoro-2-methyl- phenyl)amino)-N- (6-methoxy-2-meth- ylpyridin-3-yl)iso- nicotinamide |
| 89 | 3-(6-methoxy-2- methylpyridin-3- yl)-1-(2-methyl-4- (trifluorometh- oxy)phenyl)-6- (trifluoromethyl)- 2,3-dihydropyrido- [3,4-d]pyrimidin- 4(1H)-one | 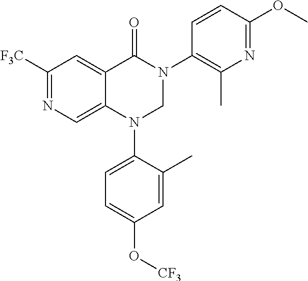 [SMILES: COc1ccc(N2CN(c3ccc(OC(F)(F)F)cc3C)c3cnc(C(F)(F)F)cc3C2=O)c(C)n1] | MS (m/z) 513.3 (M + H)⁺ | N-(6-methoxy-2- methylpyridin-3-yl)- 5-((2-methyl-4- (trifluoromethoxy) phenyl)amino)-2- (trifluoromethyl)iso- nicotinamide |
| 90 | 1-(3,4-difluoro-2- methylphenyl)-3- (6-methoxy-2-meth- ylpyridin-3-yl)-6- (trifluoromethyl)- 2,3-dihydropyrido- [3,4-d]pyrimidin- 4(1H)-one | 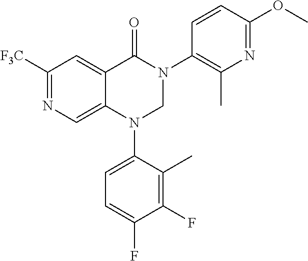 [SMILES: COc1ccc(N2CN(c3ccc(F)c(F)c3C)c3cnc(C(F)(F)F)cc3C2=O)c(C)n1] | MS (m/z) 465.3 (M + H)⁺ | 5-((3,4-difluoro-2- methylphenyl)- amino)-N-(6-meth- oxy-2-methylpyr- idin-3-yl)-2-(tri- fluoromethyl)iso- nicotinamide |
| 91 | 7-chloro-1-(4- fluoro-2-methyl- phenyl)-3-(6-meth- oxy-2-methylpyr- idin-3-yl)-2,3- dihydropyrido[4,3- d]pyrimidin-4(1H)- one | 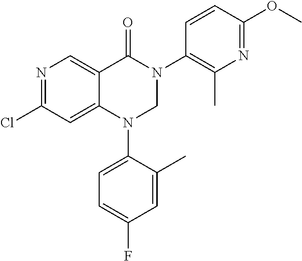 [SMILES: COc1ccc(N2CN(c3ccc(F)cc3C)c3cc(Cl)ncc3C2=O)c(C)n1] | MS (m/z) 413.4 (M + H)⁺ | 6-chloro-4-((4- fluoro-2-methyl- phenyl)amino)-N- (6-methoxy-2-meth- ylpyridin-3-yl)nico- tinamide |
| 92 | 1-(2-ethyl-4- fluorophenyl)-3-(6- methoxy-2-meth- ylpyridin-3-yl)-7- (trifluoromethyl)- 2,3-dihydropyrido [4,3-d]pyrimidin- 4(1H)-one | 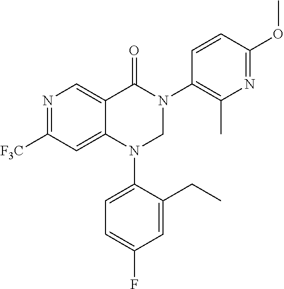 [SMILES: CCc1cc(F)ccc1N1CN(c2ccc(OC)nc2C)C(=O)c2cnc(C(F)(F)F)cc21] | MS (m/z) 461.3 (M + H)⁺ | 4-((2-ethyl-4-fluoro- phenyl)amino)-N-(6- methoxy-2-methyl- pyridin-3-yl)-6- (trifluoromethyl)- nicotinamide |
| 93 | 1-(2-ethyl-3,4- difluorophenyl)-3- (6-methoxy-2-meth- ylpyridin-3-yl)-6- (trifluoromethyl)- 2,3-dihydropyrido- [3,4-d]pyrimidin- 4(1H)-one | 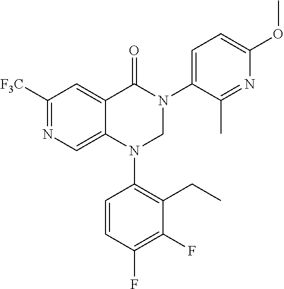 [SMILES: CCc1c(N2CN(c3ccc(OC)nc3C)C(=O)c3cc(C(F)(F)F)ncc32)ccc(F)c1F] | MS (m/z) 479.0 (M + H)⁺ | 5-((2-ethyl-3,4- difluorophenyl)- amino)-N-(6-meth- oxy-2-methylpyr- idin-3-yl)-2-(tri- fluoromethyl)iso- nicotinamide |
| 94 | 3-(5-fluoro-6-meth- oxy-2-methylpyr- idin-3-yl)-1-(2- methyl-4-(trifluoro- methoxy)phenyl)-6- (trifluoromethyl)- 2,3-dihydropyrido- [3,4-d]pyrimidin- 4(1H)-one | 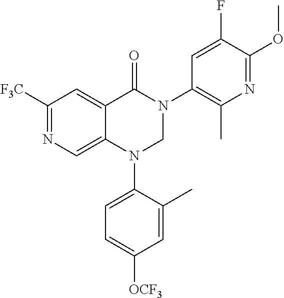 [SMILES: COc1nc(C)c(N2CN(c3ccc(OC(F)(F)F)cc3C)c3cnc(C(F)(F)F)cc3C2=O)cc1F] | MS (m/z) 531.1 (M + H)⁺ | N-(5-fluoro-6- methoxy-2-methyl- pyridin-3-yl)-5-((2- methyl-4-(trifluoro- methoxy)phenyl)- amino)-2-(trifluoro- methyl)isonicotin- amide |
| 95 | 1-(4-fluoro-2-meth- ylphenyl)-3-(5- fluoro-6-methoxy- 2-methylpyridin-3- yl)-6-(trifluorometh- methyl)-2,3- dihydropyrido [3,4- d]pyrimidin-4(1H)- one | 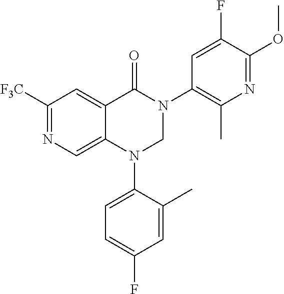 [SMILES: COc1nc(C)c(N2CN(c3ccc(F)cc3C)c3cnc(C(F)(F)F)cc3C2=O)cc1F] | MS (m/z) 465.2 (M + H)⁺ | 5-((4-fluoro-2-meth- ylphenyl)amino)-N- (5-fluoro-6-meth- oxy-2-methylpyr- idin-3-yl)-2-(tri- fluoromethyl)iso- nicotinamide |
| 96 | 1-(2-methyl-4-(tri- fluoromethoxy)- phenyl)-3-(3-meth- ylpyridin-4-yl)-6- (trifluoromethyl)- 2,3-dihydropyrido- [3,4-d]pyrimidin- 4(1H)-one | 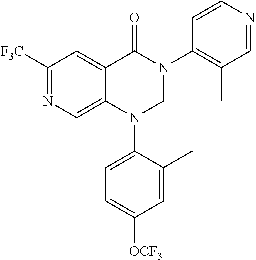 [SMILES: Cc1cnccc1N1CN(c2ccc(OC(F)(F)F)cc2C)c2cnc(C(F)(F)F)cc2C1=O] | MS (m/z) 483.3 (M + H)⁺ | 5-((2-methyl-4- (trifluoromethoxy)- phenyl)amino)-N- (3-methylpyridin-4- yl)-2-(trifluorometh- yl)isonicotinamide |

### Intermediate 97

### 1-(4-Fluoro-2-isopropylphenyl)-6-hydroxy-3-(6-methoxy-2-methylpyridin-3-yl)-2,3-dihydropyrido[3,2-d]pyrimidin-4(1H)-one

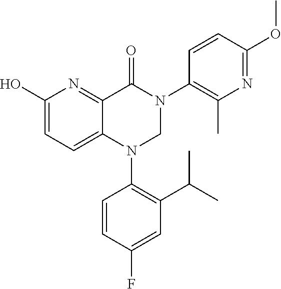 [SMILES: COc1ccc(N2CN(c3ccc(F)cc3C(C)C)c3ccc(O)nc3C2=O)c(C)n1]

To a stirring solution of 3-((4-fluoro-2-isopropylphenyl)amino)-6-hydroxy-N-(6-methoxy-2-methylpyridin-3-yl)picolinamide (290 mg, 0.707 mmol) in toluene (5 mL), PTSOH (134 mg, 0.707 mmol) and paraformaldehyde (849 mg, 28.3 mmol) were added under N₂ and the reaction mixture was stirred at 100° C. for 1 h. The reaction mixture was allowed to cool to RT, quenched with saturated NaHCO₃ (15 mL) and extracted with EtOAc (2×20 mL). The combined organic extracts were washed with water (30 mL) and brine (30 mL), dried over Na₂SO₄, and concentrated in vacuo. The residue was purified via column chromatography (Isolera, 25 g Si₂O SNAP column, 2-5% MeOH/DCM over 20 minutes) to give the title compound as a yellow solid (180 mg, 0.418 mmol, 59.2% yield). MS (m/z) 423.2 (M+H)⁺.

### Intermediate 98

### 6-Chloro-1-(4-fluoro-2-isopropylphenyl)-3-(6-methoxy-2-methylpyridin-3-yl)-2,3-dihydropyrido[3,2-d]pyrimidin-4(1H)-one

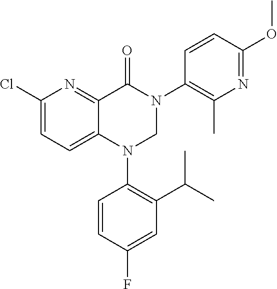 [SMILES: COc1ccc(N2CN(c3ccc(F)cc3C(C)C)c3ccc(Cl)nc3C2=O)c(C)n1]

POCl₃ (0.8 mL, 8.58 mmol) was added to 1-(4-fluoro-2-isopropylphenyl)-6-hydroxy-3-(6-methoxy-2-methylpyridin-3-yl)-2,3-dihydropyrido[3,2-d]pyrimidin-4(1H)-one (80 mg, 0.189 mmol) at 0° C. under N₂ and the reaction mixture was stirred at 90° C. for 18 h. The reaction was cooled to 0° C. and more of POCl₃ (0.1 mL, 1.073 mmol) was added. The reaction was stirred for 1 h at 90° C. and then allowed to cool to RT. The reaction was slowly poured into a solution of saturated NaHCO₃ (15 mL) at 0° C. and extracted with EtOAc (2×20 mL). The combined organic extracts were dried over Na₂SO₄, filtered, and concentrated in vacuo to give the crude title compound as a pale yellow liquid (90 mg). MS (m/z) 441.1 (M+H)⁺.

### Intermediate 99

### 1-(4-Fluoro-2-methylphenyl)-3-(6-methoxy-2-methylpyridin-3-yl)-4-oxo-1,2,3,4-tetrahydropyrido[4,3-d]pyrimidine-7-carbonitrile

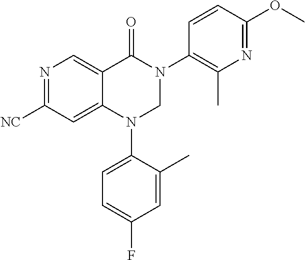 [SMILES: COc1ccc(N2CN(c3ccc(F)cc3C)c3cc(C#N)ncc3C2=O)c(C)n1]

To a solution of 7-chloro-1-(4-fluoro-2-methylphenyl)-3-(6-methoxy-2-methylpyridin-3-yl)-2,3-dihydropyrido[4,3-d]pyrimidin-4(1H)-one (80 mg, 0.194 mmol) in DMF (2 mL) were added dicyanozinc (45.5 mg, 0.388 mmol) and tetrakis (44.8 mg, 0.039 mmol). The reaction mixture was heated in a microwave oven at 150° C. for 1 h. The crude material was purified by column chromatography (Isco, 10-70% EtOAc/hexanes) to give the title product as an off-white solid (66 mg, 0.162 mmol, 84% yield). MS (m/z) 404.4 (M+H)⁺.

### Intermediate 100

### 5-((3,4-Difluoro-2-methylphenyl)amino)-2-(trifluoromethyl)isonicotinic acid, hydrochloride

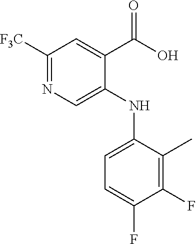 [SMILES: Cc1c(Nc2cnc(C(F)(F)F)cc2C(=O)O)ccc(F)c1F]

To a mixture of methyl 5-bromo-2-(trifluoromethyl)isonicotinate (14 g, 49.3 mmol) and 3,4-difluoro-2-methylaniline (8.47 g, 59.1 mmol) in 1,4-dioxane (224 mL) was added cesium carbonate (32.1 g, 99 mmol), then Pd₂(dba)₃ (2.257 g, 2.465 mmol) and 2,2′-bis(diphenylphosphaneyl)-1,1′-binaphthalene (3.07 g, 4.93 mmol). The mixture was stirred at 100° C. under nitrogen overnight. The reaction was cooled and then filtered through celite. The crude solution was heated at 65° C. with 5N sodium hydroxide (49.3 mL, 246 mmol) for 40 m. The solution was cooled, and 75 mL of 6N HCl was added and allowed to stir to give a yellow suspension. This was filtered to give solid, a second crop was also collected and the solids combined and slurried with 100 mL diethyl ether, sonicated and filtered to give a lemon yellow solid, which was dried for 18 h under vacuum to provide 5-((3,4-difluoro-2-methylphenyl)amino)-2-(trifluoromethyl)isonicotinic acid, hydrochloride as a yellow solid (13.25 g, 35.9 mmol, 72.9% yield). MS (m/z) 331.1 (M+H)⁺. ¹H NMR (400 MHz, DMSO-d₆) b 9.47 (s, 1H) 8.06-8.16 (m, 1H) 7.97-8.06 (m, 1H) 7.30-7.41 (m, 1H) 7.18-7.31 (m, 1H) 3.37 (br s, 3H) 2.17 (s, 3H).

### Intermediate 101

### 5-((3,4-Difluoro-2-methylphenyl)amino)-N-(6-methoxy-2-methylpyridin-3-yl)-2-(trifluoromethyl)isonicotinamide

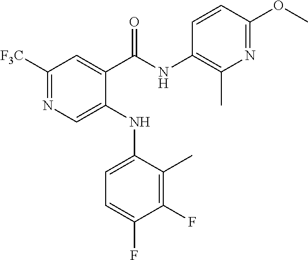 [SMILES: COc1ccc(NC(=O)c2cc(C(F)(F)F)ncc2Nc2ccc(F)c(F)c2C)c(C)n1]

A mixture of 5-((3,4-difluoro-2-methylphenyl)amino)-2-(trifluoromethyl)isonicotinic acid hydrochloride (19.75 g, 53.6 mmol), 6-methoxy-2-methylpyridin-3-amine (9.84 g, 71.2 mmol) and pyridine (23.8 mL, 295 mmol) was stirred in ethyl acetate (179 mL) to give a solution at 25° C., then T3P (88 mL, 149 mmol, 50% solution in ethyl acetate) was added and the reaction stirred at 25° C. for 2 h, then filtered. The filtrate was treated with 150 mL 1N HCl and 50 mL EtOAc, the organic separated, washed with 150 mL sat'd sodium bicarb solution, dried over anhydrous sodium sulfate, and concentrated in vacuo to a light brown solid. This solid was triturated at 70° C. in 100 mL MTBE, cooled to 25° C., and filtered to give 14.21 g of crude product. The filtrate was evaporated in vacuo to a dark oily solid and again triturated with ˜30 mL MTBE and filtered to give 4 g of a white solid product. The filtrate was concentrated in vacuo and again triturated with 15 mL MTBE to give 0.6 g more product and the solids combined to give a peach solid of title compound (19 g total, 42.0 mmol, 78% yield). The remaining filtrate was evaporated in vacuo to a dark brown gum (4.4 g) and was preabsorbed on silica. The residue was purified via column chromatography (Isco CombiFlash Rf, 0% to 30% gradient of EtOAc in Heptane; 80 g RediSep column). The pure fractions were collected and an additional 2.9 g (6.4 mmol, 12% yield) of title product isolated by concentration in vacuo as a peach solid. MS (m/z) 453.1 (M+H)⁺. ¹H NMR (400 MHz, DMSO-d₆) b 10.44 (s, 1H) 9.50 (s, 1H) 8.23 (d, J=15.16 Hz, 2H) 7.64 (d, J=8.80 Hz, 1H) 7.29-7.37 (m, 1H) 7.16-7.29 (m, 1H) 6.71 (d, J=8.31 Hz, 1H) 3.86 (s, 3H) 2.37 (s, 3H) 2.17 (s, 3H).

### Intermediate 102

### 1-(3,4-Difluoro-2-methylphenyl)-3-(6-methoxy-2-methylpyridin-3-yl)-6-(trifluoromethyl)-2,3-dihydropyrido[3,4-d]pyrimidin-4(1H)-one

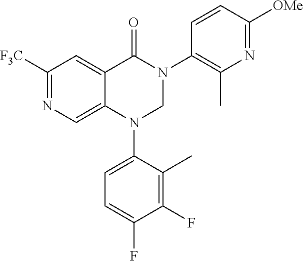 [SMILES: COc1ccc(N2CN(c3ccc(F)c(F)c3C)c3cnc(C(F)(F)F)cc3C2=O)c(C)n1]

A mixture of 5-((3,4-difluoro-2-methylphenyl)amino)-N-(6-methoxy-2-methylpyridin-3-yl)-2-(trifluoromethyl)isonicotinamide (18.37 g, 40.6 mmol) and cesium carbonate (79 g, 244 mmol) in acetonitrile (625 ml) was treated with diiodomethane (32.7 ml, 406 mmol) in a 2 L flask with reflux condenser heating at Tj=99° C. This was allowed to stir for 18 h. The reaction was not complete, so diiodomethane (9.81 ml, 122 mmol) was added and allowed to reflux for 4 h more, then cooled, filtered through a celite pad and concentrated in vacuo. The resulting solid was dissolved in dichloromethane and loaded onto 35 g of silica and dry loaded onto Isco CombiFlash Rf for silica gel chromatography (0% to 30% EtOAc in Heptane, 25 m gradient; 330 g RediSep column). The pure fractions were collected and the product isolated by concentration in vacuo as a light yellow solid, 14.7 g. This powder was dissolved in 30 mL DCM to load and purify via silica gel chromatography on an Isco CombiFlash Rf (0% to 25% gradient over 25 m of EtOAc in Heptane; 330 g RediSep column). The pure fractions were collected and the product isolated by concentration in vacuo to the title compound as a tan powder (13.7 g, 29.5 mmol, 72.6% yield). MS (m/z) 465.1 (M+H)⁺. ¹H NMR (400 MHz, DMSO-d₆) b 8.07 (s, 1H) 7.84-8.01 (m, 1H) 7.66 (br d, J=8.31 Hz, 1H) 7.39-7.53 (m, 1H) 7.31-7.38 (m, 1H) 6.75 (d, J=9.29 Hz, 1H) 4.92-5.68 (m, 2H) 3.85 (s, 3H) 2.28-2.38 (m, 3H) 2.25 (s, 3H).

### Example 1

### 1-(4-Fluoro-2-methylphenyl)-3-(2-methyl-6-oxo-1,6-dihydropyridin-3-yl)-7-(trifluoromethyl)-2,3-dihydropyrido[3,2-d]pyrimidin-4(1H)-one

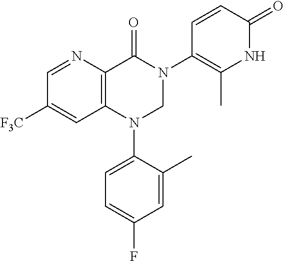 [SMILES: Cc1cc(F)ccc1N1CN(c2ccc(=O)[nH]c2C)C(=O)c2ncc(C(F)(F)F)cc21]

Iodotrimethylsilane (578 mg, 2.89 mmol) was added dropwise to a stirring solution of 1-(4-fluoro-2-methylphenyl)-3-(6-methoxy-2-methylpyridin-3-yl)-7-(trifluoromethyl)-2,3-dihydropyrido[3,2-d]pyrimidin-4(1H)-one (430 mg, 0.963 mmol) in acetonitrile (10 mL) at RT under N₂. The reaction mixture was stirred at 60° C. for 12 h, cooled to RT and concentrated under reduced pressure. The crude product was purified by reverse phase HPLC (Sunfire C18 (19×150 mm) 5 μm column, 0.1% formic acid in water/ACN) to give the title compound as a white solid (120 mg, 0.275 mmol, 28.5% yield). ¹H NMR (400 MHz, DMSO-d₆) δ: 11.80 (s, 1H), 8.53 (s, 1H), 7.43-7.38 (m, 2H), 7.33 (dd, J=2.8, 9.6 Hz, 1H), 7.25-7.15 (m, 1H), 6.92-6.75 (m, 1H), 6.21 (d, J=9.6 Hz, 1H), 5.60-4.80 (m, 2H), 2.25 (s, 3H), 2.13 (s, 3H). MS (m/z) 433.0 (M+H)⁺.

Examples 2-8 were prepared from the indicated Intermediates by methods analogous to those described for Example 1.

| Ex. | Name | Structure | Characterization | Intermediate |
|---|---|---|---|---|
| 2 | 1-(4-fluoro-2- methylphenyl)-3- (2-methyl-6-oxo- 1,6-dihydropyr- idin-3-yl)-7-(tri- fluoromethyl)- 2,3-dihydropyr- imido-[4,5-d]pyr- imidin-4(1H)-one | 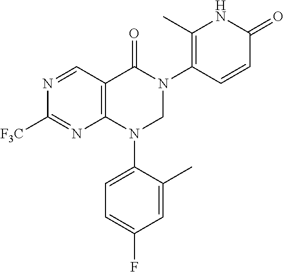 [SMILES: Cc1cc(F)ccc1N1CN(c2ccc(=O)[nH]c2C)C(=O)c2cnc(C(F)(F)F)nc21] | ¹H NMR (400 MHz, DMSO-d₆) δ: 11.85 (br s, 1H), 8.92 (d, J = 4.4 Hz, 1H), 7.51-7.39 (m, 2H), 7.35-7.25 (m, 1H), 7.18 (td, J = 8.5, 3.1 Hz, 1H), 6.27-6.18 (m, 1H), 5.58 (d, J = 9.6 Hz, 0.5H), 5.34 (d, J = 9.9 Hz, 0.5H), 5.23 (d, J = 9.9 Hz, 0.5H), 5.02 (d, J = 9.6 Hz, 0.5H), 2.23 (d, J = 2.1 Hz, 3H), 2.17-2.13 (m, 3H). MS (m/z) 434.0 (M + H)⁺ | 1-(4-fluoro-2- methylphenyl)-3-(6- methoxy-2-methyl- pyridin-3-yl)-7-(tri- fluoromethyl)-2,3- dihydropyrimido- [4,5-d]pyrimidin- 4(1H)-one |
| 3 | 6-chloro-1-(4- fluoro-2-isopro- pylphenyl)-3-(2- methyl-6-oxo-1,6- dihydropyridin- 3-yl)-2,3-dihydro- pyrido-[3,4-d]pyr- imidin-4(1H)-one | 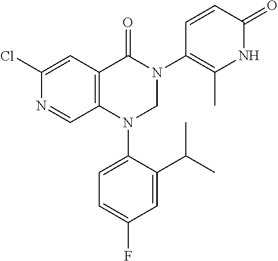 [SMILES: Cc1[nH]c(=O)ccc1N1CN(c2ccc(F)cc2C(C)C)c2cnc(Cl)cc2C1=O] | ¹H NMR (400 MHz, DMSO-d₆) δ: 11.81 (br s, 1H), 7.70 (s, 1H), 7.45-7.30 (m, 4H), 7.25-7.15 (m, 1H), 6.21 (d, J = 9.3 Hz, 1H), 5.49 (d, J = 9.6 Hz, 0.6H), 5.20 (d, J = 10.1 Hz, 0.4H), 5.02 (d, J = 10.1 Hz, 0.4H), 4.74 (d, J = 9.5 Hz, 0.6H), 3.24- 3.08 (m, 1H), 2.13-2.09 (m, 3H), 1.23-1.11 (m, 6H). MS (m/z) 427.0 (M + H)⁺ | 6-chloro-1-(4- fluoro-2-isopropyl- phenyl)-3-(6-meth- oxy-2-methylpyr- idin-3-yl)-2,3- dihydropyrido[3,4- d]pyrimidin-4(1H)- one |
| 4 | 1-(4-fluoro-2- methylphenyl)-3- (2-methyl-6-oxo- 1,6-dihydropyr- idin-3-yl)-7-(tri- fluoromethyl)- 2,3-dihydropyr- ido[4,3-d]pyrim- idin-4(1H)-one | 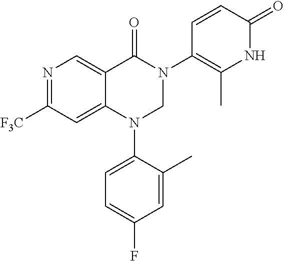 [SMILES: Cc1cc(F)ccc1N1CN(c2ccc(=O)[nH]c2C)C(=O)c2cnc(C(F)(F)F)cc21] | ¹H NMR (400 MHz, DMSO-d₆) δ: 11.81 (s, 1H), 8.89 (s, 1H), 7.53- 7.47 (m, 1H), 7.43-7.37 (m, 2H), 7.28-7.22 (m, 1H), 6.36 (d, J = 12.80 Hz, 1H), 6.22 (d, J = 8.40 Hz, 1H), 5.55 (d, J = 9.20 Hz, 0.6H), 5.28 (d, J = 10.00 Hz, 0.4H), 5.16 (d, J = 10.00 Hz, 0.4H), 4.90 (d, J = 9.60 Hz, 0.6H), 2.26 (s, 3H), 2.14 (s, 3H). MS (m/z) 432.8 (M + H)⁺. | 1-(4-fluoro-2-meth- ylphenyl)-3-(6- methoxy-2-meth- ylpyridin-3-yl)-7- (trifluoromethyl)- 2,3-dihydropyrido- [4,3-d]pyrimidin- 4(1H)-one |
| 5 | 1-(4-fluoro-2- isopropylphenyl)- 3-(2-methyl-6- oxo-1,6-dihydro- pyridin-3-yl)-6- (trifluoromethyl)- 2,3-dihydropyr- ido[3,4-d]pyrim- idin-4(1H)-one | 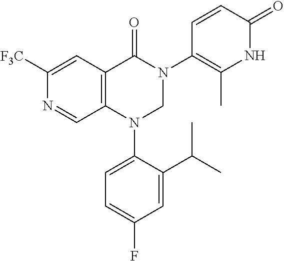 [SMILES: Cc1[nH]c(=O)ccc1N1CN(c2ccc(F)cc2C(C)C)c2cnc(C(F)(F)F)cc2C1=O] | ¹H NMR (400 MHz, DMSO-d₆) δ: 11.84 (br s, 1H), 8.02 (s, 1H), 7.69 (d, J = 17.6 Hz, 1H), 7.51-7.46 (m, 1H), 7.46- 7.32 (m, 2H), 7.28-7.20 (m, 1H), 6.22 (d, J = 9.6 Hz, 1H), 5.56 (d, J = 9.5 Hz, 0.6H), 5.29 (d, J = 10.1 Hz, 0.4H), 5.10 (d, J = 10.0 Hz, 0.4H), 4.84 (d, J = 9.5 Hz, 0.6H), 3.24-3.06 (m, 1H), 2.16- 2.09 (m, 3H), 1.29-1.10 (m, 6H). MS (m/z) 461.2 (M + H)⁺ | 1-(4-fluoro-2-iso- propylphenyl)-3-(6- methoxy-2-methyl- pyridin-3-yl)-6-(tri- fluoromethyl)-2,3- dihydropyrido[3,4- d]pyrimidin-4(1H)- one |
| 6 | 1-(4-fluoro-2- methylphenyl)-3- (2-methyl-6-oxo- 1,6-dihydropyr- idin-3-yl)-6-(tri- fluoromethyl)- 2,3-dihydropyr- ido-[3,4-d]pyrim- idin-4(1H)-one | 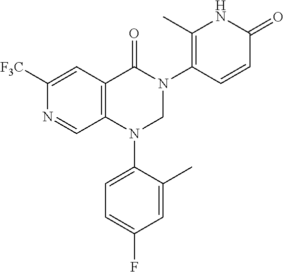 [SMILES: Cc1cc(F)ccc1N1CN(c2ccc(=O)[nH]c2C)C(=O)c2cc(C(F)(F)F)ncc21] | ¹H NMR (400 MHz, DMSO-d₆) δ: 11.82 (br s, 1H), 8.02 (s, 1H), 7.82- 7.68 (m, 1H), 7.48 (dd, J = 8.8, 5.5 Hz, 1H), 7.41-7.32 (m, 2H), 7.27- 7.17 (m, 1H), 6.21 (d, J = 9.6 Hz, 1H), 5.51 (d, J = 9.6 Hz, 0.5H), 5.21 (br s, 1H), 4.91 (d, J = 9.9 Hz, 0.5H), 2.28 (s, 3H), 2.16-2.06 (m, 3H). MS (m/z) 433.0 (M + H)⁺ | 1-(4-fluoro-2-meth- ylphenyl)-3-(6- methoxy-2-methyl- pyridin-3-yl)-6- (trifluoromethyl)- 2,3-dihydropyrido- [3,4-d]pyrimidin- 4(1H)-one |
| 7 | 1-(4-fluoro-2- methylphenyl)-3- (2-methyl-6-oxo- 1,6-dihydropyr- idin-3-yl)-7-(tri- fluoromethyl)- 2,3-dihydropter- idin-4(1H)-one | 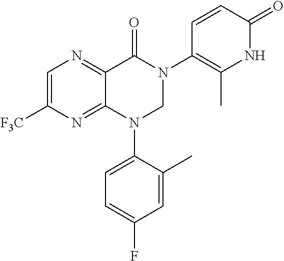 [SMILES: Cc1cc(F)ccc1N1CN(c2ccc(=O)[nH]c2C)C(=O)c2ncc(C(F)(F)F)nc21] | ¹H NMR (400 MHz, DMSO-d₆) ) [mixture of rotamers] δ: 11.85 (s, 1H), 8.59 (s, 1H), 7.45- 7.38 (m, 2H), 7.26 (dd, J = 2.40, 9.80 Hz, 1H), 7.16 (td, J = 2.80, 8.40 Hz, 1H), 6.25-6.23 (m, 1H), 5.62 (d, J = 10.00 Hz, 0.5H), 5.37 (d, J = 9.60 Hz, 0.5H), 5.21 (d, J = 9.60 Hz, 0.5H), 4.99 (d, J = 9.60 Hz, 0.5H), 2.21 (s, 3H), 2.15-2.10 (m, 3H). MS (m/z) 434.0 (M + H)⁺ | 1-(4-fluoro-2-meth- ylphenyl)-3-(6- methoxy-2-meth- ylpyridin-3-yl)-7- (trifluoromethyl)- 2,3-dihydropteridin- 4(1H)-one |
| 8 | 1-(4-fluoro-2- methylphenyl)-3- (2-methyl-6-oxo- 1,6-dihydropyr- idin-3-yl)-6-(tri- fluoromethyl)-2,3- dihydropteridin- 4(1H)-one | 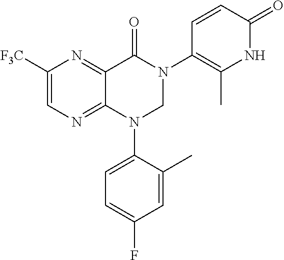 [SMILES: Cc1cc(F)ccc1N1CN(c2ccc(=O)[nH]c2C)C(=O)c2nc(C(F)(F)F)cnc21] | ¹H NMR (400 MHz, DMSO-d6) ) [mixture of rotamers] δ: 11.84 (s, 1H), 8.65 (s, 1H), 7.44- 7.38 (m, 2H), 7.27 (d, J = 9.60 Hz, 1H), 7.16 (td, J = 3.20, 8.60 Hz, 1H), 6.25-6.21 (m, 1H), 5.60 (d, J = 9.60 Hz, 0.5H), 5.37 (d, J = 10.40 Hz, 0.5H), 5.21 (d, J = 10.00 Hz, 0.5H), 5.00 (d, J = 9.60 Hz, 0.5H), 2.24 (s, 3H), 2.20-2.15 (m, 3H). MS (m/z) 434.0 (M + H)⁺ | 1-(4-fluoro-2-meth- ylphenyl)-3-(6- methoxy-2-meth- ylpyridin-3-yl)-6- (trifluoromethyl)- 2,3-dihydropteridin- 4(1H)-one |

### Example 9

### 6-Chloro-1-(4-fluoro-2-isopropylphenyl)-3-(2-methyl-6-oxo-1,6-dihydropyridin-3-yl)-2,3-dihydropyrido[3,2-d]pyrimidin-4(1H)-one

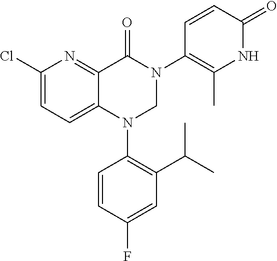 [SMILES: Cc1[nH]c(=O)ccc1N1CN(c2ccc(F)cc2C(C)C)c2ccc(Cl)nc2C1=O]

To a stirring solution of 6-chloro-1-(4-fluoro-2-isopropylphenyl)-3-(6-methoxy-2-methylpyridin-3-yl)-2,3-dihydropyrido[3,2-d]pyrimidin-4(1H)-one (90 mg, 0.102 mmol) in DMF (1 mL) were added p-toluenesulfonic acid monohydrate (97 mg, 0.508 mmol) and lithium chloride (21.52 mg, 0.508 mmol) at 0° C. under N₂. The reaction mixture was stirred at 120° C. for 2 h and then cooled to 25° C. The reaction was quenched with ice water (15 mL) and extracted with EtOAc (2×20 mL). The combined organic extracts were washed with brine (20 mL), dried over Na₂SO₄, filtered and concentrated under reduced pressure. The resultant brown gum was purified by reverse phase HPLC (X-bridge C18 (19×150 mm) 5 μm column, 10 mM NH₄HCO₃ in water/ACN) to give the title compound as an off-white solid (16 mg, 0.037 mmol, 36.5% yield). ¹H NMR (400 MHz, DMSO-d₆) δ: 11.80 (br s, 1H), 7.43-7.28 (m, 4H), 7.23-7.15 (m, 1H), 6.76-6.66 (m, 1H), 6.21 (d, J=9.3 Hz, 1H), 5.48 (d, J=9.6 Hz, 0.6H), 5.22 (d, J=10.4 Hz, 0.4H), 4.97 (d, J=10.4 Hz, 0.4H), 4.72 (d, J=9.6 Hz, 0.6H), 3.24-3.03 (m, 1H), 2.18-2.03 (m, 3H), 1.23-1.08 (m, 6H). MS (m/z) 427.2 (M+H)⁺.

Examples 10-11 was prepared from the indicated intermediate by methods analogous to those described for Example 9.

| Ex. | Name | Structure | Characterization | Intermediate |
|---|---|---|---|---|
| 10 | 1-(2-ethyl-3,4- difluorophenyl)-3- (2-methyl-6-oxo- 1,6-dihydropyr- idin-3-yl)-6-(tri- fluoromethyl)-2,3- dihydropyrido[3,4- d]pyrimidin-4(1H)- one | 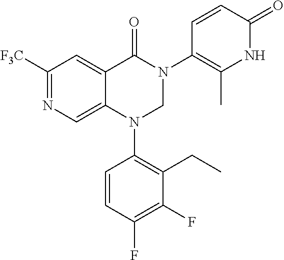 [SMILES: CCc1c(N2CN(c3ccc(=O)[nH]c3C)C(=O)c3cc(C(F)(F)F)ncc32)ccc(F)c1F] | ¹H NMR (CHLOROFORM- d, 400 MHz) δ: 13.14 (br d, J = 11.7 Hz, 1H), 8.27 (s, 1H), 7.87 (br d, J = 9.8 Hz, 1H), 7.40-7.30 (m, 1H), 7.21 (br d, J = 9.0 Hz, 1H), 7.10-7.00 (m, 1H), 6.50 (d, J = 9.5 Hz, 1H), 5.39 (br d, J = 9.0 Hz, 0.5H), 5.15 (br d, J = 10.8 Hz, 0.5H), 4.97 (br d, J = 10.0 Hz, 0.5H), 4.73 (br d, J = 9.5 Hz, 0.5H), 2.90-2.70 (m, 2H), 2.36 (br d, 3H, J = 19.1 Hz), 1.30- 1.20 (m, 3H). MS (m/z) 465.0 (M + H)⁺ | 1-(2-ethyl-3,4- difluorophenyl)- 3-(6-methoxy-2- methylpyridin-3- yl)-6-(trifluoro- methyl)-2,3- dihydropyrido- [3,4-d]pyrimidin- 4(1H)-one |
| 11 | 3-(5-fluoro-2- methyl-6-oxo-1,6- dihydropyridin-3- yl)-1-(4-fluoro-2- methylphenyl)-6- (trifluoromethyl)- 2,3-dihydropyrido- [3,4-d]pyrimidin- 4(1H)-one | 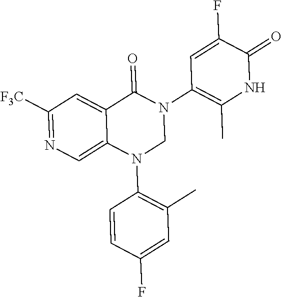 [SMILES: Cc1cc(F)ccc1N1CN(c2cc(F)c(=O)[nH]c2C)C(=O)c2cc(C(F)(F)F)ncc21] | ¹H NMR (DMSO-d₆, 400 MHz) δ: 12.38 (br s, 1H), 8.03 (s, 1H), 7.7-7.8 (m, 1H), 7.51 (d, J = 11.2 Hz, 1H), 7.4-7.5 (m, 1H), 7.34 (br d, J = 9.8 Hz, 1H), 7.1-7.3 (m, 1H), 5.2-5.5 (m, 1H), 4.9-5.2 (m, 1H), 2.28 (s, 3H), 2.10 (br d, J = 14.7 Hz, 3H) MS (m/z) 451.2 (M + H)⁺ | 1-(4-fluoro-2- methylphenyl)- 3-(5-fluoro-6- methoxy-2-meth- ylpyridin-3-yl)- 6-(trifluorometh- yl)-2,3-dihydro- pyrido[3,4-d]pyr- imidin-4(1H)-one |

### Example 12

### 1-(4-Fluoro-2-methylphenyl)-3-(2-methyl-6-oxo-1,6-dihydropyridin-3-yl)-2,3-dihydropyrido[3,4-d]pyrimidin-4(1H)-one

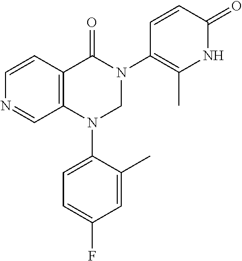 [SMILES: Cc1cc(F)ccc1N1CN(c2ccc(=O)[nH]c2C)C(=O)c2ccncc21]

To a mixture of 1-(4-fluoro-2-methylphenyl)-3-(6-methoxy-2-methylpyridin-3-yl)-2,3-dihydropyrido[3,4-d]pyrimidin-4(1H)-one (250 mg, 0.661 mmol) and sodium iodide (990 mg, 6.61 mmol) in acetonitrile (10.00 mL) was added chlorotrimethylsilane (0.838 mL, 6.61 mmol) and the reaction was stirred at 60° C. for 3 h. The reaction was diluted with DCM (30 mL), washed with aq. Na₂S₂O₃, dried over Na₂SO₄, filtered and concentrated under reduced pressure. The crude product was purified by MDAP (XSELECT CSH C18 (150 mm×30 mm) 5 μm column, A=0.1% v/v solution of formic acid in water, B=0.1% v/v solution of formic acid in acetonitrile, 5-35% B, gradient time 3-12 min) to give the title compound as a white solid (76 mg, 0.198 mmol, 30.0% yield). ¹H NMR (400 MHz, DMSO-d₆) δ: 11.80 (br s, 1H), 8.19 (d, J=4.89 Hz, 1H), 7.73 (d, J=4.89 Hz, 1H), 7.65 (br s, 1H), 7.44-7.34 (m, 2H), 7.31 (dd, J=9.29, 2.93 Hz, 1H), 7.22-7.13 (m, 1H), 6.20 (d, J=9.78 Hz, 1H), 5.60-4.73 (m, 2H), 2.27 (s, 3H), 2.09 (br s, 3H). MS (m/z) 365.3 (M+H)⁺.

### Example 17

### 1-(3,4-Difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one

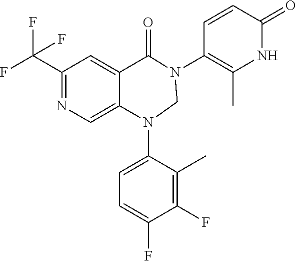 [SMILES: Cc1[nH]c(=O)ccc1N1CN(c2ccc(F)c(F)c2C)c2cnc(C(F)(F)F)cc2C1=O]

A solution of 1-(3,4-difluoro-2-methylphenyl)-3-(6-methoxy-2-methylpyridin-3-yl)-6-(trifluoromethyl)-2,3-dihydropyrido[3,4-d]pyrimidin-4(1H)-one (13.7 g, 29.5 mmol) and HCl (59.0 ml, 295 mmol, 5N in isopropanol)) in isopropanol (73.8 ml) was heated at reflux (Tj=91° C.) for 12 h then 6 h at room temperature. The mixture was concentrated in vacuo to an orange foamy oil, then the oil was partitioned between 100 mL each EtOAc and aqueous saturated sodium bicarbonate solution, the aqueous layer was back extracted with 50 mL EtOAc, the combined organics were washed with brine, dried over anhydrous sodium sulfate, and concentrated in vacuo to a light brown foam. The foam was dissolved in 15 mL dichloromethane, and after 30 m, solid precipitated and was filtered to give title compound as a white solid (6.85 g, 15.2 mmol, 51.6% yield). The filtrate containing crude product was collected and was purified via silica gel chromatography on an Isco CombiFlash Rf (1:1 ethyl acetate:heptane to 70% 3:1 EtOAc:EtOH; 330 g RediSep column over 20 min). The pure fractions were collected and the product isolated by concentration in vacuo to give more title compound (4.1 g, 9.1 mmol, 30.9% yield) after drying under high vacuum 18 h as an off white solid. MS (m/z) 451.1 (M+H)⁺. ¹H NMR (700 MHz, DMSO-d₆) b ppm 11.18-12.29 (m, 1H) 8.00-8.07 (m, 1H) 7.79-7.96 (m, 1H) 7.43-7.51 (m, 1H) 7.37-7.43 (m, 1H) 7.29-7.36 (m, 1H) 6.21 (d, J=9.47 Hz, 1H) 4.90-5.55 (m, 2H) 2.19-2.29 (m, 3H) 2.02-2.16 (m, 3H).

### Example 17a

### 1-(3,4-Difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one Crystalline Form Preparation

### Form 1

2.0 g of 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one was added to a 40 mL vial. To this was added 14 mL (7 vols) of MeOH and the contents shaken at 25° C. and 750 rpm. The temperature was increased to 50° C., and full dissolution occurred in about 15 mins. Solids began to form at about 10 mins following dissolution. These solids were allowed to grow in for about 10 mins to give a slurry. The temperature was then increased to 63° C. and held for 4 hrs. The contents were then cooled to 25° C. at a rate of 0.1° C./min and held for 1 hr. The crystals were then ripened by again heating to 63° C. at a rate of 1.0° C./min and held for 4 hrs, before finally cooling again to 25° C. at a rate of 0.1° C./min and holding overnight. The contents were filtered to remove the crystalline solid and placed in a vacuum oven at 60° C. overnight to afford 1.52 g of crystalline 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Pattern 1—see XRPD and DSC, Table 1 and FIGS. 1 and 2).

### Form 2

70 mg of 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Form 3) was added to each of 12×2 mL HPLC vials. 500 μL of various solvents were added to each of the vials. The vials were then stirred at 750 rpm starting at 25° C. The temperature was increased to 50° C. In those vials that appeared to show a physical change in the input material, the solid was filtered off and run on XRPD. Those solvents that showed physical changes in the input material were: isopropanol, isopropyl acetate and 1-butanol. The crystalline material isolated from these solvents seem to generate an XRPD of Pattern 2 (see XRPD and DSC, Table 1 and FIGS. 3 and 4).

### Form 3

1.0 g of amorphous (chromatographed, rotary-evaporated) 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one was added to a 20 mL vial. 5 mL (5 vols) of EtOH was added, and full dissolution of the input was achieved at 25° C. A clarifying filtration was done and the mother liquor added into a separate clean 20 mL vial. The vial was uncapped and left out to evaporate over the course of three days. The contents were slurried in 1 mL of EtOH and filtered to recover the crystalline 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Pattern 3—see XRPD, Table 1 and FIG. 5).

### Form 4

500 mg of 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one was added to a 20 mL vial. 7.5 mL of MeCN (15 vols) was added to the vial, and the contents shaken at 25° C. and 750 rpm. Full dissolution of the input material occurred in about 5 mins. The temperature was increased to 30° C., and solid particles began to form within 5 minutes. Mixing was continued at this temperature for another 30 minutes, during which time that the contents became a thicker but well mixing slurry. The temperature was increased to 63° C. at a rate of 1.0° C./min. The contents were then held at this temperature for 4 hrs. After 4 hrs at 63° C., the temperature was decreased to 25° C. at a rate of 0.1° C./min and holding there for 1 hr. The temperature was then cycled back up to 63° C. at a rate of 1.0° C./min and held there for 4 hrs, before finally cooling again to 25° C. at a rate of 0.1° C./min and holding there overnight. The following morning the contents were filtered and aspirated until a mist. The crystalline solid was then placed in a vacuum oven at 60° C. overnight to afford 368 mg of crystalline 1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one (Pattern 4—see XRPD and DSC, Table 1 and FIGS. 6 and 7).

### XRPD—Characteristic Peaks and d-Spacings for Forms 1 to 4 when Measured Using Cu Kα Radiation

TABLE 1

| Form 1 | | Form 2 | | Form 3 | | Form 4 | |
|---|---|---|---|---|---|---|---|
| | d-spac- | | d-spac- | | d-spac- | | d-spac- |
| 2θ/° | ing/Å | 2θ/° | ing/Å | 2θ/° | ing/Å | 2θ/° | ing/Å |
| 6.3 | 14.0 | 4.9 | 18.0 | 7.08 | 12.5 | 3.88 | 22.8 |
| 7.36 | 12.0 | 6.92 | 12.8 | 9.28 | 9.6 | 6.96 | 12.7 |
| 8.04 | 11.0 | 8.2 | 10.8 | 10.2 | 8.7 | 7.32 | 12.1 |
| 9.04 | 9.8 | 10.8 | 8.2 | 13.84 | 6.4 | 7.6 | 11.6 |
| 9.12 | 9.7 | 13.5 | 6.6 | 15 | 6.0 | 8.96 | 9.9 |
| 9.96 | 8.9 | 14.76 | 6.0 | 16.28 | 5.5 | 9.16 | 9.7 |
| 11.24 | 7.9 | 16.1 | 5.6 | 17.64 | 5.1 | 10.16 | 8.7 |
| 12.6 | 7.1 | 16.76 | 5.3 | 19.76 | 4.6 | 12.44 | 7.2 |
| 13.4 | 6.6 | 17.3 | 5.2 | 20.76 | 4.3 | 13.96 | 6.4 |
| 14.6 | 6.1 | 17.9 | 5.0 | 22.76 | 4.0 | 14.72 | 6.1 |
| 15.44 | 5.8 | 20 | 4.5 | 24.56 | 3.7 | 15.36 | 5.8 |
| 15.92 | 5.6 | 21.4 | 4.2 | 26.32 | 3.5 | 16.88 | 5.3 |
| 16.44 | 5.4 | 23 | 3.9 | 26.92 | 3.4 | 18.2 | 4.9 |
| 16.8 | 5.3 | 23.95 | 3.8 | 27.72 | 3.3 | 19.24 | 4.7 |
| 17.16 | 5.2 | 25.8 | 3.5 | 28.52 | 3.2 | 19.96 | 4.5 |
| 18.44 | 4.9 | 26.8 | 3.4 | | | 20.76 | 4.3 |
| 18.76 | 4.8 | 27.6 | 3.3 | | | 21.56 | 4.2 |
| 19.36 | 4.6 | 29.05 | 3.2 | | | 22.4 | 4.0 |
| 19.96 | 4.5 | 30.15 | 3.1 | | | 22.84 | 4.0 |
| 20.6 | 4.4 | | | | | 24.04 | 3.8 |
| 21.2 | 4.3 | | | | | 24.96 | 3.7 |
| 21.92 | 4.1 | | | | | 25.4 | 3.6 |
| 22.92 | 4.0 | | | | | 25.92 | 3.5 |
| 23.88 | 3.8 | | | | | 26.28 | 3.5 |
| 24.52 | 3.7 | | | | | 26.92 | 3.4 |
| 25.12 | 3.6 | | | | | 27.92 | 3.3 |
| 25.56 | 3.6 | | | | | 28.36 | 3.2 |
| 26 | 3.5 | | | | | 29.4 | 3.1 |
| 26.72 | 3.4 | | | | | 29.84 | 3.1 |
| 27.52 | 3.3 | | | | | 30 | 3.1 |
| 27.88 | 3.3 | | | | | 30.96 | 3.0 |
| 28.32 | 3.2 | | | | | 31.84 | 2.9 |
| 30.28 | 3.1 | | | | | 35.84 | 2.6 |
| 31 | 3.0 | | | | | | |
| 31.52 | 2.9 | | | | | | |
| 32.36 | 2.9 | | | | | | |
| 34.48 | 2.7 | | | | | | |
| 34.84 | 2.7 | | | | | | |
| 36.12 | 2.6 | | | | | | |
| 36.86 | 2.6 | | | | | | |
| 38.05 | 2.5 | | | | | | |

Examples 14-16 & 18-21 were prepared from the indicated Intermediate by methods analogous to those described for Example 12.

| Ex. | Name | Structure | Characterization | Intermediate |
|---|---|---|---|---|
| 14 | 1-(2-ethyl-4-fluoro- phenyl)-3-(2-methyl- 6-oxo-1,6-dihydro- pyridin-3-yl)-6- (trifluoromethyl)- 2,3-dihydropyrido- [3,4-d]pyrimidin- 4(1H)-one | 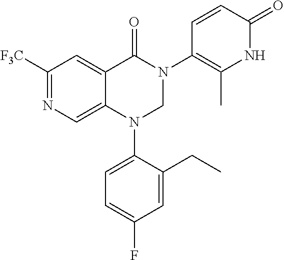 [SMILES: CCc1cc(F)ccc1N1CN(c2ccc(=O)[nH]c2C)C(=O)c2cc(C(F)(F)F)ncc21] | ¹H NMR (400 MHz,DMSO- d₆) δ: 11.83 (br s, 1H), 8.01 (s, 1H), 7.71 (d, J = 18.6 Hz, 1H), 7.54-7.48 (m, 1H), 7.38 (br dd, J = 13.0, 9.5 Hz, 2H), 7.29-7.17 (m, 1H), 6.22 (d, J = 9.8 Hz, 1H), 5.56 (d, J = 9.3 Hz, 0.6H), 5.29 (d, J = 10.3 Hz, 0.4H), 5.12 (d, J = 10.3 Hz, 0.4H), 4.87 (d, J = 9.3 Hz, 0.6H), 2.63 (q, J = 6.8 Hz, 2H), 2.12 (d, J = 8.8 Hz, 3H), 1.16 (q, J = 7.3 Hz, 3H). MS (m/z) 447.2 (M + H)⁺ | 1-(2-ethyl-4-fluoro- phenyl)-3-(6-methoxy- 2-methylpyridin-3-yl)- 6-(trifluoromethyl)- 2,3-dihydropyrido[3,4- d]pyrimidin-4(1H)-one |
| 15 | 6-chloro-1-(4-fluoro- 2-methylphenyl)-3- (2-methyl-6-oxo- 1,6-dihydropyridin- 3-yl)-2,3-dihydro- pyrido[3,4-d]pyrim- idin-4(1H)-one | 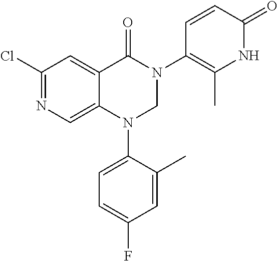 [SMILES: Cc1cc(F)ccc1N1CN(c2ccc(=O)[nH]c2C)C(=O)c2cc(Cl)ncc21] | ¹H NMR (400 MHz, DMSO- d₆) δ: 11.80 (br s, 1H), 7.71 (s, 1H), 7.45 (br s, 1H), 7.39-7.35 (m, 2H), 7.31-7.28 (m, 1H), 7.18-7.15 (m, 1H), 6.19 (d, J = 9.8 Hz, 1H), 5.41-4.84 (m, 2H), 2.25 (s, 3H), 2.08 (br s, 3H). MS (m/z) 399.3 (M + H). | 6-chloro-1-(4-fluoro- 2-methylphenyl)-3-(6- methoxy-2-methyl- pyridin-3-yl)-2,3- dihydropyrido[3,4-d]- pyrimidin-4(1H)-one |
| 16 | 1-(2-methyl-4- (trifluoromethoxy)- phenyl)-3-(2-meth- yl-6-oxo-1,6- dihydropyridin-3- yl)-6-(trifluoro- methyl)-2,3-dihydro- pyrido[3,4-d]pyrim- idin-4(1H)-one | 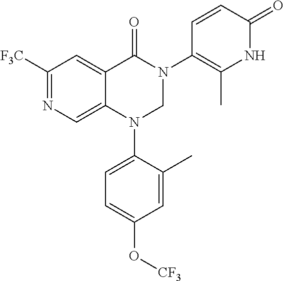 [SMILES: Cc1cc(OC(F)(F)F)ccc1N1CN(c2ccc(=O)[nH]c2C)C(=O)c2cc(C(F)(F)F)ncc21] | ¹H NMR (400 MHz, DMSO- d₆) δ: 11.82 (br s, 1H), 8.05 (s, 1H), 7.80 (d, J = 32 Hz, 1H), 7.56 (d, J = 8.3 Hz, 1H), 7.50 (br s, 1H), 7.44- 7.35 (m, 2H), 6.21 (d, J = 9.8 Hz, 1H), 5.60-5.95 (m, 2H), 2.31 (s, 3H), 2.0-2.2 (m, 3H). MS (m/z) 499.3 (M + H)⁺ | 3-(6-methoxy-2-meth- ylpyridin-3-yl)-1-(2- methyl-4-(trifluoro- methoxy)phenyl)-6- (trifluoromethyl)-2,3- dihydropyrido[3,4-d]- pyrimidin-4(1H)-one |
| 17 | 1-(3,4-difluoro-2- methylphenyl)-3- (2-methyl-6-oxo- 1,6-dihydropyridin- 3-yl)-6-(trifluoro- methyl)-2,3-dihydro- pyrido[3,4-d]pyrim- idin-4(1H)-one | 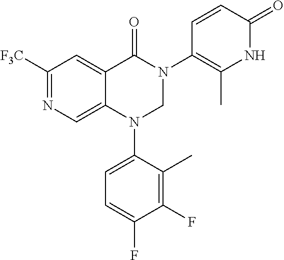 [SMILES: Cc1[nH]c(=O)ccc1N1CN(c2ccc(F)c(F)c2C)c2cnc(C(F)(F)F)cc2C1=O] | ¹H NMR (400 MHz, DMSO- d₆) δ: 11.82 (br s, 1H), 8.04 (s, 1H), 7.90 (d, J = 32 Hz, 1H), 7.3-7.6 (m, 3H), 6.21 (d, J = 9.3 Hz, 1H), 5.55- 4.90 (m, 2H), 2.23 (s, 3H), 2.10 (d, J = 24 Hz, 3H). MS (m/z) 451.3 (M + H)⁺ | 5-((3,4-difluoro-2- methylphenyl)amino)- N-(6-methoxy-2-meth- ylpyridin-3-yl)-2-(tri- fluoromethyl)isonico- tinamide |
| 18 | 7-chloro-1-(4- fluoro-2-methyl- phenyl)-3-(2-meth- yl-6-oxo-1,6- dihydropyridin- 3-yl)-2,3-dihydro- pyrido[4,3-d]pyrim- idin-4(1H)-one | 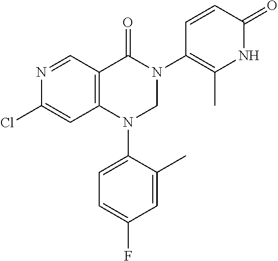 [SMILES: Cc1cc(F)ccc1N1CN(c2ccc(=O)[nH]c2C)C(=O)c2cnc(Cl)cc21] | ¹H NMR (400 MHz, DMSO- d₆) δ: 11.80 (m, 1H), 7.71 (s, 1H), 7.45 (br s, 1H), 7.42-7.34 (m, 2H), 7.30 (dd, J = 2.7, 9.5 Hz, 1H), 7.17 (br t, J = 7.1 Hz, 1H), 6.21 (d, J = 9.8 Hz, 1H), 5.56- 4.69 (m, 2H), 2.26 (s, 3H), 2.09 (br s, 3H). MS (m/z) 399.2 (M + H)⁺ | 7-chloro-1-(4-fluoro- 2-methylphenyl)-3- (6-methoxy-2-methyl- pyridin-3-yl)-2,3- dihydropyrido[4,3-d]- pyrimidin-4(1H)-one |
| 19 | 1-(4-fluoro-2-meth- ylphenyl)-3-(2- methyl-6-oxo-1,6- dihydropyridin-3- yl)-4-oxo-1,2,3,4- tetrahydropyrido- [4,3-d]pyrimidine- 7-carbonitrile | 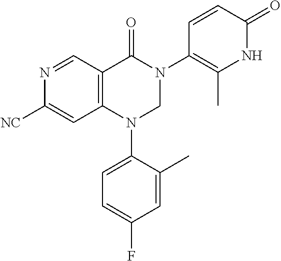 [SMILES: Cc1cc(F)ccc1N1CN(c2ccc(=O)[nH]c2C)C(=O)c2cnc(C#N)cc21] | ¹H NMR (400 MHz, DMSO- d₆) δ: 11.80 (br s, 1 H), 8.20 (s, 1 H), 7.69 (br d, J = 19.56 Hz, 1 H), 7.53-7.45 (m, 1 H), 7.44-7.32 (m, 2 H), 7.22 (br dd, J = 7.34, 3.91 Hz, 1 H), 6.21 (br d, J = 9.78 Hz, 1 H), 5.53 (br d, J = 9.78 Hz, 0.5 H), 5.28-5.13 (m, 1 H), 4.93 (br d, J = 9.78 Hz, 0.5 H) 2.26 (s, 3 H), 2.11 (br d, J = 7.34 Hz, 3 H) MS (m/z) 390.3 (M + H)⁺ | 1-(4-fluoro-2-methyl- phenyl)-3-(6-methoxy- 2-methylpyridin-3-yl)- 4-oxo-1,2,3,4-tetra- hydropyrido[4,3-d]- pyrimidine-7-car- bonitrile |
| 20 | 1-(2-ethyl-4-fluoro- phenyl)-3-(2-meth- yl-6-oxo-1,6- dihydropyridin- 3-yl)-7-(trifluoro- methyl)-2,3-dihydro- pyrido[4,3-d]pyrim- idin-4(1H)-one | 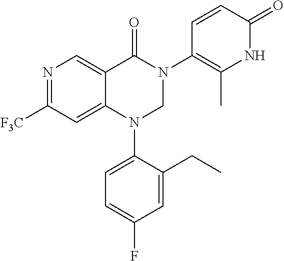 [SMILES: CCc1cc(F)ccc1N1CN(c2ccc(=O)[nH]c2C)C(=O)c2cnc(C(F)(F)F)cc21] | ¹H NMR (400 MHz, DMSO- d₆) δ: 11.83 (br. s, 1H), 8.89 (s, 1H), 7.51 (dt, J = 5.6, 8.2 Hz, 1H), 7.50-7.30 (m, 2H), 7.2-7.3 (m, 1H), 6.34 (d, J = 6.8 Hz, 1H), 6.22 (dd, J = 4.2, 9.5 Hz, 1H), 5.60 (d, J = 9.8 Hz, 0.6H), 5.36 (d, J = 10.3 Hz, 0.4H), 5.10 (d, J = 9.8 Hz, 0.4H), 4.87 (d, J = 9.3 Hz, 0.6H), 2.70-2.50 (m, 2H), 2.14 (d, 3H, J = 5.9 Hz), 1.20-1.10 (m, 3H) MS (m/z) 447.3 (M + H)⁺ | 1-(2-ethyl-4-fluoro- phenyl)-3-(6-methoxy- 2-methylpyridin-3-yl)- 7-(trifluoromethyl)- 2,3-dihydropyrido[4,3- d]pyrimidin-4(1H)-one |
| 21 | 3-(5-fluoro-2-meth- yl-6-oxo-1,6- dihydropyridin-3- yl)-1-(2-methyl-4- (trifluoromethoxy)- phenyl)-6-(tri- fluoromethyl)-2,3- dihydropyrido[3,4- d]pyrimidin-4(1H)- one | 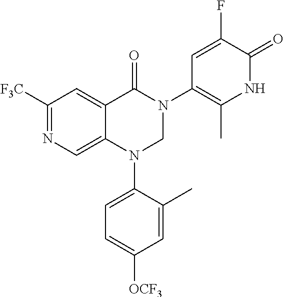 [SMILES: Cc1cc(OC(F)(F)F)ccc1N1CN(c2cc(F)c(=O)[nH]c2C)C(=O)c2cc(C(F)(F)F)ncc21] | ¹H NMR (400 MHz, DMSO- d₆) δ: 12.37 (br s, 1H), 8.05 (s, 1H), 7.86-7.78 (m, 1H), 7.57-7.49 (m, 3H), 7.37 (br s, 1H), 5.52-5.00 (m, 2H), 2.31 (s, 3H), 2.10 (br s, 3H) MS (m/z) 517.1 (M + H)⁺ | 3-(5-fluoro-6-methoxy- 2-methylpyridin-3-yl)- 1-(2-methyl-4-(tri- fluoromethoxy)phenyl)- 6-(trifluoromethyl)- 2,3-dihydropyrido[3,4- d]pyrimidin-4(1H)-one |

### Example 22

### 1-(4-Fluoro-2-methylphenyl)-3-(2-methyl-6-oxo-1,6-dihydropyridin-3-yl)-4-oxo-1,2,3,4-tetrahydropyrido[3,4-d]pyrimidine-6-carbonitrile

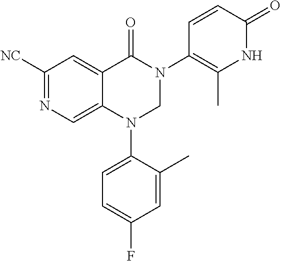 [SMILES: Cc1cc(F)ccc1N1CN(c2ccc(=O)[nH]c2C)C(=O)c2cc(C#N)ncc21]

To a solution of 6-chloro-1-(4-fluoro-2-methylphenyl)-3-(2-methyl-6-oxo-1,6-dihydropyridin-3-yl)-2,3-dihydropyrido[3,4-d]pyrimidin-4(1H)-one (75 mg, 0.188 mmol) in DMF (2 mL) were added dicyanozinc (44.2 mg, 0.376 mmol) and tetrakis (43.5 mg, 0.038 mmol). The reaction mixture was heated at 150° C. overnight and then heated in a microwave at 150° C. for 1 h. The reaction mixture was cooled, filtered through Celite® and purified by MDAP (XSELECT CSH C18 (150 mm×30 mm) 5 μm column, A=0.1% v/v solution of formic acid in water, B=0.1% v/v solution of formic acid in acetonitrile, 15-55% B, gradient time 3-22 min) to give the title product as an off-white solid (50 mg, 0.127 mmol, 67.6% yield). ¹H NMR (400 MHz, DMSO-d₆) δ: 11.83 (br s, 1H), 8.19 (br s, 1H), 7.69 (br d, J=19.6 Hz, 1H), 7.49 (dd, J=5.4, 8.8 Hz, 1H), 7.42-7.32 (m, 2H), 7.29-7.17 (m, 1H), 6.21 (br. d, J=9.8 Hz, 1H), 5.53 (br. d, J=9.8 Hz, 0.5H), 5.32-5.16 (m, 1H), 4.93 (br. d, J=9.8 Hz, 0.5H), 2.26 (s, 3H), 2.11 (br d, J=7.3 Hz, 3H). MS (m/z) 390.3 (M+H)⁺.

### Example 23

### 3-Methyl-4-(1-(2-methyl-4-(trifluoromethoxy)phenyl)-4-oxo-6-(trifluoromethyl)-1,4-dihydropyrido[3,4-d]pyrimidin-3(2H)-yl)pyridine 1-oxide

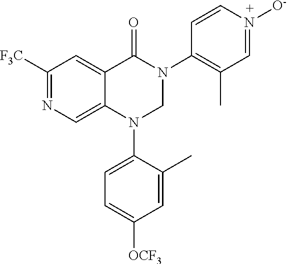

A mixture of 1-(2-methyl-4-(trifluoromethoxy)phenyl)-3-(3-methylpyridin-4-yl)-6-(trifluoromethyl)-2,3-dihydropyrido[3,4-d]pyrimidin-4(1H)-one (38 mg, 0.079 mmol) and mCPBA (26.5 mg, 0.118 mmol) in DCM (1.50 mL) was stirred at 0° C. for 4 h and then concentrated under vacuum. The residue was purified by MDAP (XSelect CSH Prep C18 um OBD, 30-85% gradient. Water with 10 mM ammonium bicarb and 0.075% ammonium hydroxide/acetonitrile, 40 mL/min flow rate, 17 min overall run time) to afford the title product as a white solid (15 mg, 0.029 mmol, 36.3% yield). ¹H NMR (400 MHz, DMSO-d₆) δ: 8.34-8.33 (m, 1H), 8.21 (dd, J=1.5, 6.8 Hz, 1H), 8.14 (s, 1H), 7.92 (br s, 1H), 7.63 (d, J=8.8 Hz, 1H), 7.55 (d, J=2.9 Hz, 1H), 7.47-7.42 (m, 2H), 5.55-5.30 (m, 2H), 2.37 (s, 3H), 2.16 (s, 3H). MS (m/z) 499.3 (M+H)⁺.

Biological Assays

The Naᵥ1.8 Inhibitor compounds or pharmaceutically acceptable salts thereof of the invention are useful for treatment of pain, pain disorders or conditions, pain-related disorders or conditions or pain caused by diseases, respectively, such as those defined throughout the instant application.

The biological activity of the compounds of the invention can be determined using suitable assays, such as those measuring such inhibition and those evaluating the ability of the compounds to inhibit voltage gated sodium channel Naᵥ 1.8 in vitro or in animal models of infection.

### Biological Assay Example 1

Human embryonic kidney 293 cells (HEK293) expressing human Naᵥ1.8, human Naᵥβ1 and human TREK1 (HEK293-Naᵥ1.8) were grown at 37° C., 5% CO₂ in 150 cm² flasks. HEK293-Naᵥ1.8 were passaged every 2-3 days into T175 cell culture flasks when confluency reached 80-90%.

Pharmacological assessment of the compounds of the invention was performed using HEK293-Nav1.8 in combination with an assay developed on the QPatch 48 HTX electrophysiological system. HEK293-Naᵥ1.8 were prepared on the day of use by removing culture media, washing in DPBS, adding Accutase (2 ml to cover the surface, aspirate 1 ml then 1.5 min at 37° C.) followed by addition of CHO-SFM II to stop the enzyme digestion and in order to obtain a suspension of 3×10⁶ cell/mL.

Compound was prepared in an extracellular solution of the following composition: NaCl (145 mM), KCl (4 mM), CaCl₂ (2 mM), MgCl₂ (2 mM), HEPES (1 mM), Glucose (10 mM), pH 7.4 with NaOH Osmolality 300 mOsM/L. The intracellular solution was used of the following composition: CsF (115 mM), CsCl (20 mM), NaCl (5 mM), EGTA (10 mM), HEPES (10 mM), Sucrose (20 mM), pH 7.2 with CsOH Osmolality 310 mOsm/L.

Utilizing the voltage-clamp mode in the QPatch 48 HTX system a half inactivation state voltage protocol (V₁/2) was used to determine pharmacological activity of compounds of the invention at Naᵥ1.8 ion channels. A V_{1/2} protocol was utilized with the following voltage steps: a holding voltage of −100 mV was established followed by a 20 ms voltage step to 0 mV (P1), followed by an inactivating voltage step at −46 mV for 8 seconds, followed by a step to −100 mV for 20 ms, before a 20 ms step to 0 mV (P2) before returning to the holding voltage of −100 mV. This voltage protocol was repeated at a frequency of 0.07 Hz., current magnitude was quantified at the P2 step throughout the recording. Inhibition of the measured current amplitude with the compounds of the invention was analyzed by fitting a 4-8 point dose-response curve allowing determination of the fifty percent inhibition concentration (IC₅₀). Within the QPatch HTX software, P2 current was normalized according to measurements made at baseline after compound and after positive reference compound and fit to the following equation:

$$n . {I}_{CPD} = Normalized Current = \frac{( Input - Baseline )}{( FullResponse - Baseline )}$$

To assess current run-down over the course of the experiment vehicle-only wells were utilized and the normalized current with vehicle-only (n.I_{VEH}) was determined. To correct the compound response for run-down, the currents were corrected according the following formula:

$$n . {I}_{RD \_ Correct} = \frac{( n . {I}_{CPD} - n . {I}_{VEH} )}{( 1 - n . {I}_{VEH} )}$$

Compounds of the invention were tested for activity against Nav1. 8 sodium channels in the above assay in one or more experimental runs and the results are shown in Table 2 below. Potency of the compounds of the invention is reported as a pIC50 value. The pIC50 value is the negative log of the IC50 value, wherein the IC50 value is the half maximal inhibitory concentration measured in molar (M). For compounds tested in more than one experimental run, the pIC50 value is reported as an average.

TABLE 2

| Example No. | [Nav1.8] pIC50 |
|---|---|
| 1 | 5.9 |
| 2 | 6 |
| 3 | 7 |
| 4 | 6.8 |
| 5 | 7.2 |
| 6 | 7 |
| 7 | 5.4 |
| 8 | 5.1 |
| 9 | 5.8 |
| 10 | 7.5 |
| 11 | 6.5 |
| 12 | 5.3 |
| 14 | 7.1 |
| 15 | 6.6 |
| 16 | 7.9 |
| 17 | 7.2 |
| 18 | 6.6 |
| 19 | 5.4 |
| 20 | 7.1 |
| 21 | 7.4 |
| 22 | 5.4 |
| 23 | 6.7 |

It is to be understood that the invention is not limited to the embodiments illustrated hereinabove and the right is reserved to the illustrated embodiments and all modifications coming within the scope of the following claims.

1.-41. (canceled)

42. A compound of formula (I): 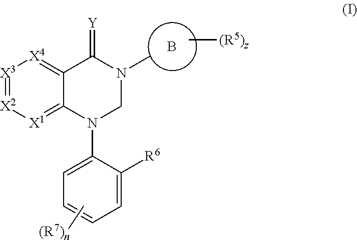
or a tautomer thereof, or a pharmaceutically acceptable salt thereof,
wherein:
Y is O or S;
X¹ is nitrogen or CR¹,
X² is nitrogen or CR²,
X³ is nitrogen or CR³, and
X⁴ is nitrogen or CR⁴,
provided at least one of X², X³, and X⁴ is nitrogen;
each of R¹, R², R³, and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

43. The compound according to claim 42 which is a compound of formula (I-A): 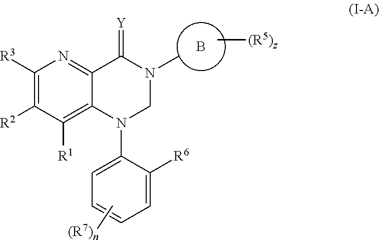
or a tautomer thereof, or a pharmaceutically acceptable salt thereof,
wherein:
Y is O or S;
each of R¹, R², and R³ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

44. The compound according to claim 42 which is a compound of formula (I-B): 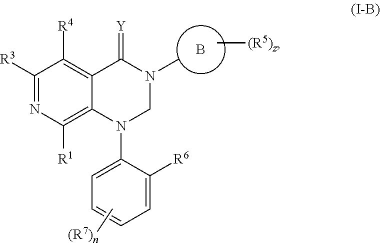
or a tautomer thereof, or a pharmaceutically acceptable salt thereof,
wherein:
Y is O or S;
each of R¹, R³, and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

45. The compound according to claim 42 which is a compound of formula (I-C): 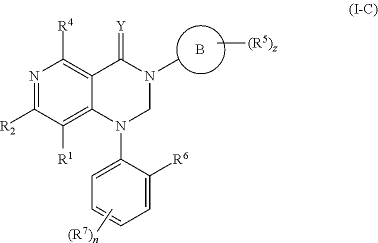
or a or a tautomer thereof, or a pharmaceutically acceptable salt thereof,
wherein:
Y is O or S;
each of R¹, R², and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

46. The compound according to claim 42 which is a compound of formula (I-D): 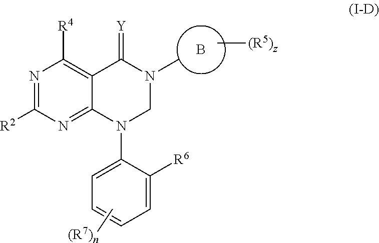
or a tautomer thereof, or a pharmaceutically acceptable salt thereof,
wherein:
Y is O or S;
each of R² and R⁴ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

47. The compound according to claim 42 which is a compound of formula (I-E): 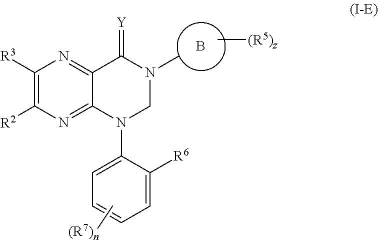
or a tautomer thereof, or a pharmaceutically acceptable salt thereof,
wherein:
Y is O or S;
each of R² and R³ is independently hydrogen, halo, cyano, —NR^{a}R^{b}, —(C₁₋₆)-alkyl, —(C₁₋₆)-haloalkyl, —O—(C₁₋₆)— alkyl, or —O—(C₁₋₆)-haloalkyl;
ring B is phenyl, or a 5- or 6-membered heterocyclyl containing 1 or 2 nitrogen ring atoms;
each R⁵ is independently halo, oxo, —OH, —NR^{a}R^{b}, —(C₁₋₆)alkyl, —(C₁₋₆)haloalkyl, —COOR^{a}, —C(O)NR^{a}R^{b}, or —S(O)ₚR^{c};
R⁶ is hydrogen, —(C₁₋₆)alkyl, —O—(C₁₋₆)-alkyl, or —NR^{a}R^{b};
each R⁷ is independently halo, —(C₁₋₆)alkyl, —O—(C₁₋₆)alkyl, or —O—(C₁₋₆)-haloalkyl;
each of R^{a} and R^{b} is independently hydrogen, —(C₁₋₆)alkyl, or —(C₁₋₆)haloalkyl;
R^{c} is hydrogen, —OH, —NR^{a}R^{b}, —(C₁₋₆)-alkyl, or —(C₁₋₆)-haloalkyl;
n is 0, 1, 2, or 3;
p is 0, 1, or 2; and
z is 0, 1, 2, or 3.

48. The compound according to claim 42, wherein Y is O.

49. The compound according to claim 42, wherein 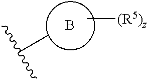
is:

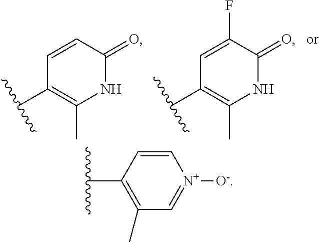

50. The compound according to claim 42, wherein R⁶ is —CH₃, —CH₂CH₃, or —CH(CH₃)₂.

51. The compound according to claim 42, wherein 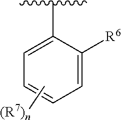
is:

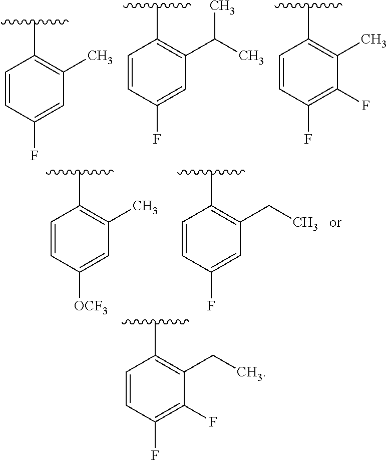

52. A compound selected from the group consisting of:
1-(4-fluoro-2-methylphenyl)-3-(2-methyl-6-oxo-1,6-dihydropyridin-3-yl)-7-(trifluoromethyl)-2,3-dihydropyrido[3,2-d]pyrimidin-4(1H)-one;
1-(3,4-difluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one;
1-(2-ethyl-3,4-difluorophenyl)-3-(2-methyl-6-oxo-1,6-dihydropyridin-3-yl)-6-(trifluoromethyl)-2,3-dihydropyrido[3,4-d]pyrimidin-4(1H)-one;
1-(4-fluoro-2-methylphenyl)-3-(2-methyl-6-oxo-1,6-dihydropyridin-3-yl)-7-(trifluoromethyl)-2,3-dihydropyrimido[4,5-d]pyrimidin-4(1H)-one;
6-chloro-1-(4-fluoro-2-isopropylphenyl)-3-(2-methyl-6-oxo-1,6-dihydropyridin-3-yl)-2,3-dihydropyrido[3,4-d]pyrimidin-4(1H)-one;
1-(4-fluoro-2-methylphenyl)-3-(2-methyl-6-oxo-1,6-dihydropyridin-3-yl)-7-(trifluoromethyl)-2,3-dihydropyrido[4,3-d]pyrimidin-4(1H)-one;
1-(4-fluoro-2-isopropylphenyl)-3-(2-methyl-6-oxo-1,6-dihydropyridin-3-yl)-6-(trifluoromethyl)-2,3-dihydropyrido[3,4-d]pyrimidin-4(1H)-one;
1-(4-fluoro-2-methylphenyl)-3-(2-methyl-6-oxo-1,6-dihydropyridin-3-yl)-6-(trifluoromethyl)-2,3-dihydropyrido[3,4-d]pyrimidin-4(1H)-one;
6-Chloro-1-(4-fluoro-2-isopropylphenyl)-3-(2-methyl-6-oxo-1,6-dihydropyridin-3-yl)-2,3-dihydropyrido[3,2-d]pyrimidin-4(1H)-one;
1-(4-Fluoro-2-methylphenyl)-3-(2-methyl-6-oxo-1,6-dihydropyridin-3-yl)-2,3-dihydropyrido[3,4-d]pyrimidin-4(1H)-one;
1-(2-ethyl-4-fluorophenyl)-3-(2-methyl-6-oxo-1,6-dihydropyridin-3-yl)-6-(trifluoromethyl)-2,3-dihydropyrido[3,4-d]pyrimidin-4(1H)-one;
1-(4-fluoro-2-methylphenyl)-3-(2-methyl-6-oxo-1,6-dihydropyridin-3-yl)-7-(trifluoromethyl)-2,3-dihydropteridin-4(1H)-one;
1-(4-fluoro-2-methylphenyl)-3-(2-methyl-6-oxo-1,6-dihydropyridin-3-yl)-6-(trifluoromethyl)-2,3-dihydropteridin-4(1H)-one;
6-chloro-1-(4-fluoro-2-methylphenyl)-3-(2-methyl-6-oxo-1,6-dihydropyridin-3-yl)-2,3-dihydropyrido[3,4-d]pyrimidin-4(1H)-one;
1-(2-methyl-4-(trifluoromethoxy)phenyl)-3-(2-methyl-6-oxo-1,6-dihydropyridin-3-yl)-6-(trifluoromethyl)-2,3-dihydropyrido[3,4-d]pyrimidin-4(1H)-one;
1-(4-Fluoro-2-methylphenyl)-3-(2-methyl-6-oxo-1,6-dihydropyridin-3-yl)-4-oxo-1,2,3,4-tetrahydropyrido[3,4-d]pyrimidine-6-carbonitrile;
7-chloro-1-(4-fluoro-2-methylphenyl)-3-(2-methyl-6-oxo-1,6-dihydropyridin-3-yl)-2,3-dihydropyrido[4,3-d]pyrimidin-4(1H)-one;
1-(4-fluoro-2-methylphenyl)-3-(2-methyl-6-oxo-1,6-dihydropyridin-3-yl)-4-oxo-1,2,3,4-tetrahydropyrido[4,3-d]pyrimidine-7-carbonitrile;
1-(2-ethyl-4-fluorophenyl)-3-(2-methyl-6-oxo-1,6-dihydropyridin-3-yl)-7-(trifluoromethyl)-2,3-dihydropyrido[4,3-d]pyrimidin-4(1H)-one;
3-(5-fluoro-2-methyl-6-oxo-1,6-dihydropyridin-3-yl)-1-(2-methyl-4-(trifluoromethoxy)phenyl)-6-(trifluoromethyl)-2,3-dihydropyrido[3,4-d]pyrimidin-4(1H)-one;
3-(5-fluoro-2-methyl-6-oxo-1,6-dihydropyridin-3-yl)-1-(4-fluoro-2-methylphenyl)-6-(trifluoromethyl)-2,3-dihydropyrido[3,4-d]pyrimidin-4(1H)-one; and
3-Methyl-4-(1-(2-methyl-4-(trifluoromethoxy)phenyl)-4-oxo-6-(trifluoromethyl)-1,4-dihydropyrido[3,4-d]pyrimidin-3(2H)-yl)pyridine 1-oxide,
or a tautomer thereof, or a pharmaceutically acceptable salt thereof.

53. A pharmaceutical composition comprising a compound according to claim 42 or a tautomer thereof, or a pharmaceutically acceptable salt thereof, and a pharmaceutically acceptable excipient.

54. A method of inhibiting a Naᵥ1.8 voltage-gated sodium channel in a subject in need thereof, the method comprising administering to the subject a compound according to claim 42, or a tautomer thereof, or a pharmaceutically acceptable salt thereof.

55. A method of treatment of pain or a pain-associated disease, disorder, or condition in a subject in need thereof, the method comprising administering to the subject a therapeutically effective amount of a compound according to claim 42 or a tautomer thereof, or a pharmaceutically acceptable salt thereof.

56. A method of treatment of atrial fibrillation in a subject in need thereof, the method comprising administering to the subject a therapeutically effective amount of a compound according to claim 42 or a tautomer thereof, or a pharmaceutically acceptable salt thereof.

57. A method of inhibiting a Naᵥ1.8 voltage-gated sodium channel in a subject in need thereof, the method comprising administering to the subject a pharmaceutical composition according to claim 53.

58. A method of treatment of pain or a pain-associated disease, disorder, or condition in a subject in need thereof, the method comprising administering to the subject a therapeutically effective amount of a pharmaceutical composition according to claim 53.

59. A method of treatment of atrial fibrillation in a subject in need thereof, the method comprising administering to the subject a therapeutically effective amount of a pharmaceutical composition according to claim 53.
